(19) United States
(12) Patent Application Publication
(10) Pub. No.: US 2026/0176637 A1
(43) Pub. Date: Jun. 25, 2026

(54) COMPOSITIONS AND METHODS FOR PRODUCING CIRCULAR POLYRIBONUCLEOTIDES

(71) Applicant: Flagship Pioneering Innovations VI, LLC, Cambridge, MA (US)

(72) Inventors: Vadim DUDKIN, Wellesley, MA (US); Ki Young PAEK, Brighton, MA (US)

(21) Appl. No.: 19/127,502

(22) PCT Filed: Nov. 8, 2023
(86) PCT No.: PCT/US2023/079058

§ 371 Date: May 6, 2025

Related U.S. Application Data
(60) Provisional application No. 63/423,711, filed on Nov. 8, 2022.

(51) Int. Cl. C12N 15/67; C12N 15/10; C12N 15/113

(52) U.S. Cl. CPC C12N 15/67; C12N 15/10; C12N 15/113; C12N 2310/12; C12N 2310/532; C12N 2830/34; C12N 2830/42; C12N 2840/203

(57) ABSTRACT

The present disclosure relates, generally, to compositions and methods for producing, purifying, and using circular RNA.

### SEQUENCE LISTING

This application contains a Sequence Listing which has been filed electronically in Extensible Markup Language (XML) format and is hereby incorporated by reference in its entirety. Said XML copy, created on Nov. 1, 2023, is named 51509-071WO2_Sequence_Listing_11_1_23.XML and is 55,374 bytes in size.

### BACKGROUND

There is a need for methods of producing, purifying, and using circular polyribonucleotides.

### SUMMARY OF THE INVENTION

The disclosure provides compositions and methods for producing, purifying, and using circular RNA.

In one aspect, the invention features a linear polyribonucleotide having the formula 5′-(A)-(B)-(C)-(D)-(E)-(F)-(G)-3′. The linear polyribonucleotide includes, from 5′ to 3′, (A) a 3′ half of Group I catalytic intron fragment from a T4 phage nrdB gene or nrdD gene; (B) a 3′ splice site; (C) a 3′ exon fragment; (D) a polyribonucleotide cargo; (E) a 5′ exon fragment; (F) a 5′ splice site; and (G) a 5′ half of Group I catalytic intron fragment from a T4 phage nrdB gene or nrdD gene. The polyribonucleotide includes a first annealing region that has from 2 to 50, e.g., 5 to 50, e.g., 6 to 50, e.g., 7 to 50, e.g., 8 to 50 (e.g., from 10 to 30, 10 to 20, or 10 to 15, e.g., at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) ribonucleotides and is present within (A) the 3′ half of Group I catalytic intron fragment from the T4 phage nrdB gene or nrdD gene; (B) the 3′ splice site; or (C) the 3′ exon fragment. The polyribonucleotide also includes a second annealing region that has from 2 to 50, e.g., 5 to 50, e.g., 6 to 50, e.g., 7 to 50, e.g., 8 to 50 (e.g., from 10 to 30, 10 to 20, or 10 to 15, e.g., at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) ribonucleotides and is present within (E) the 5′ exon fragment; (F) the 5′ splice site; or (G) the 5′ half of Group I catalytic intron fragment from the T4 phage nrdB gene or nrdD gene. The first annealing region has from 80% to 100% (e.g., 85% to 100%, e.g., 90% to 100%, e.g., 80%, 85%, 90%, 95%, 97%, 99%, or 100%) complementarity with the second annealing region or has from zero to 10 (e.g., 0, 1, 2, 3, 4, 5, 6, 7, 8, 9, or 10), mismatched base pairs.

In another aspect, the invention features a linear polyribonucleotide having the formula 5′-(A)-(B)-(C)-(D)-(E)-(F)-(G)-3′. The linear polyribonucleotide includes, from 5′ to 3′, (A) a 3′ half of Group I catalytic intron fragment from a T4 phage nrdB gene; (B) a 3′ splice site; (C) a 3′ exon fragment; (D) a polyribonucleotide cargo; (E) a 5′ exon fragment; (F) a 5′ splice site; and (G) a 5′ half of Group I catalytic intron fragment from a T4 phage nrdB gene. The polyribonucleotide includes a first annealing region that has from 2 to 50, e.g., 5 to 50, e.g., 6 to 50, e.g., 7 to 50, e.g., 8 to 50 (e.g., from 10 to 30, 10 to 20, or 10 to 15, e.g., at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) ribonucleotides and is present within (A) the 3′ half of Group I catalytic intron fragment from the T4 phage nrdB gene; (B) the 3′ splice site; or (C) the 3′ exon fragment. The polyribonucleotide also includes a second annealing region that has from 2 to 50, e.g., 5 to 50, e.g., 6 to 50, e.g., 7 to 50, e.g., 8 to 50 (e.g., from 10 to 30, 10 to 20, or 10 to 15, e.g., at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) ribonucleotides and is present within (E) the 5′ exon fragment; (F) the 5′ splice site; or (G) the 5′ half of Group I catalytic intron fragment from the T4 phage nrdB gene. The first annealing region has from 80% to 100% (e.g., 85% to 100%, e.g., 90% to 100%, e.g., 80%, 85%, 90%, 95%, 97%, 99%, or 100%) complementarity with the second annealing region or has from zero to 10 (e.g., 0, 1, 2, 3, 4, 5, 6, 7, 8, 9, or 10), mismatched base pairs.

In another aspect, the invention features a linear polyribonucleotide having the formula 5′-(A)-(B)-(C)-(D)-(E)-(F)-(G)-3′. The linear polyribonucleotide includes, from 5′ to 3′, (A) a 3′ half of Group I catalytic intron fragment from a T4 phage nrdD gene; (B) a 3′ splice site; (C) a 3′ exon fragment; (D) a polyribonucleotide cargo; (E) a 5′ exon fragment; (F) a 5′ splice site; and (G) a 5′ half of Group I catalytic intron fragment from a T4 phage nrdD gene. The polyribonucleotide includes a first annealing region that has from 2 to 50, e.g., 5 to 50, e.g., 6 to 50, e.g., 7 to 50, e.g., 8 to 50 (e.g., from 10 to 30, 10 to 20, or 10 to 15, e.g., at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) ribonucleotides and is present within (A) the 3′ half of Group I catalytic intron fragment from the T4 phage nrdD gene; (B) the 3′ splice site; or (C) the 3′ exon fragment. The polyribonucleotide also includes a second annealing region that has from 2 to 50, e.g., 5 to 50, e.g., 6 to 50, e.g., 7 to 50, e.g., 8 to 50 (e.g., from 10 to 30, 10 to 20, or 10 to 15, e.g., at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) ribonucleotides and is present within (E) the 5′ exon fragment; (F) the 5′ splice site; or (G) the 5′ half of Group I catalytic intron fragment from the T4 phage nrdD gene. The first annealing region has from 80% to 100% (e.g., 85% to 100%, e.g., 90% to 100%, e.g., 80%, 85%, 90%, 95%, 97%, 99%, or 100%) complementarity with the second annealing region or has from zero to 10 (e.g., 0, 1, 2, 3, 4, 5, 6, 7, 8, 9, or 10), mismatched base pairs.

In some embodiments, (A) or (C) includes the first annealing region and (E) or (G) includes the second annealing region.

In some embodiments, the 3′ exon fragment of (C) includes the first annealing region and the 5′ exon fragment of (E) includes the second annealing region.

In some embodiments, the 3′ exon fragment of (C) includes the first annealing region and the 5′ half of Group I catalytic intron fragment of (G) includes the second annealing region.

In some embodiments, the 3′ half of Group I catalytic intron fragment of (A) includes the first annealing region and the 5′ exon fragment of (E) includes the second annealing region.

In some embodiments, first annealing region and the second annealing region include zero or one mismatched base pair.

In some embodiments, the first annealing region and the second annealing region are 100% complementary.

In some embodiments, the first annealing region includes from 6 to 30 ribonucleotides and the second annealing region includes from 6 to 30 ribonucleotides.

In some embodiments, the first annealing region includes from 8 to 20 ribonucleotides and the second annealing region includes from 8 to 20 ribonucleotides.

In some embodiments, the first annealing region includes from 8 to 17 ribonucleotides and the second annealing region includes from 8 to 17 ribonucleotides.

In some embodiments, the first annealing region includes from 10 to 15 ribonucleotides and the second annealing region includes from 13 to 17 ribonucleotides.

In some embodiments, the 3′ half of Group I catalytic intron fragment of (A) is from the T4 phage nrdB gene.

In some embodiments, the 3′ half of Group I catalytic intron fragment of (A) includes a sequence having at least 80% sequence identity to 5′-TTGCAAAACAAGGTTCAACGACTAGTCTTCGGACGTAGGGTCAAGCGACTCGAAATGGGGAGAATC CCTCCGGGATTGTGATATAGTCTGGACTGCATGGTAACATGCAGCAGTTCATAAGAGAACGGGTTGA GAATTAGCGAGCTCAATCGAACATACG-3′ (SEQ ID NO: 2).

In some embodiments, the 3′ half of Group I catalytic intron fragment of (A) includes a sequence having at least 85%, 90%, 95%, 97%, 99%, or 100% sequence identity to 5′-TTGCAAAACAAGGTTCAACGACTAGTCTTCGGACGTAGGGTCAAGCGACTCGAAATGGGGAGAATC CCTCCGGGATTGTGATATAGTCTGGACTGCATGGTAACATGCAGCAGTTCATAAGAGAACGGGTTGA GAATTAGCGAGCTCAATCGAACATACG-3′ (SEQ ID NO: 2).

In some embodiments, the 5′ half of Group I catalytic intron fragment of (G) is from the T4 phage nrdB gene. In some embodiments, the 3′ half of Group I catalytic intron fragment of (A) is from the T4 phage nrdB gene and the 5′ half of Group I catalytic intron fragment of (G) is from the T4 phage nrdB gene.

In some embodiments, the 5′ half of Group I catalytic intron of (G) includes a sequence having at least 80% sequence identity to 5′-AAAATGCGCCTTTAAACGGTAACGTTTATCGAAAACTCCTTTAATTGCTGGAAAGTCCTTTATGGAAA ACTAGCAGCCAAGGTTTTGCTT-3′ (SEQ ID NO: 6).

In some embodiments, the 5′ half of Group I catalytic intron of (G) includes a sequence having at least 85%, 90%, 95%, 97%, 99%, or 100% sequence identity to 5′-AAAATGCGCCTTTAAACGGTAACGTTTATCGAAAACTCCTTTAATTGCTGGAAAGTCCTTTATGGAAA ACTAGCAGCCAAGGTTTTGCTT-3′ (SEQ ID NO: 6).

In some embodiments, the 3′ half of Group I catalytic intron fragment of (A) is from the T4 phage nrdD gene.

In some embodiments, the 3′ half of Group I catalytic intron fragment of (A) includes a sequence having at least 80% sequence identity to 5′-CAGTAGCTGTAAATGCCCAACGACTATCCCTGATGAATGTAAGGGAGTAGGGTCAAGCGACCCGAA ACGGCAGACAACTCTAAGAGTTGAAGATATAGTCTGAACTGCATGGTGACATGCAGCTGTTTATCCT CGTATAAATATGAATACGAGGTGAAACGATGAAATGAATTACATTGTTTCATATAAACGGGTAGAGAA GTAGCGAACTCTACTGAACACATTG-3′ (SEQ ID NO: 10).

In some embodiments, the 3′ half of Group I catalytic intron fragment of (A) includes a sequence having at least 85%, 90%, 95%, 97%, 99%, or 100% sequence identity to 5′-CAGTAGCTGTAAATGCCCAACGACTATCCCTGATGAATGTAAGGGAGTAGGGTCAAGCGACCCGAA ACGGCAGACAACTCTAAGAGTTGAAGATATAGTCTGAACTGCATGGTGACATGCAGCTGTTTATCCT CGTATAAATATGAATACGAGGTGAAACGATGAAATGAATTACATTGTTTCATATAAACGGGTAGAGAA GTAGCGAACTCTACTGAACACATTG-3′ (SEQ ID NO: 10).

In some embodiments, the 5′ half of Group I catalytic intron fragment of (G) is from the T4 phage nrdD gene. In some embodiments, the 3′ half of Group I catalytic intron fragment of (A) is from the T4 phage nrdD gene and the 5′ half of Group I catalytic intron fragment of (G) is from the T4 phage nrdD gene.

In some embodiments, the 5′ half of Group I catalytic intron of (G) includes a sequence having at least 80% sequence identity to 5′-TAACGTAAGTCAAGCTCATGTAAAATCTGCCTAAAACGGGAAACTCTCACTGAGACAATCCGTTGCTA AATCAG-3′ (SEQ ID NO: 14).

In some embodiments, the 5′ half of Group I catalytic intron of (G) includes a sequence having at least 85%, 90%, 95%, 97%, 99%, or 100% sequence identity to 5′-TAACGTAAGTCAAGCTCATGTAAAATCTGCCTAAAACGGGAAACTCTCACTGAGACAATCCGTTGCTA AATCAG-3′ (SEQ ID NO: 14).

In some embodiments, the 3′ exon fragment of (C) includes a sequence having at least 80% sequence identity to 5′-GTACCTTTAACTTCCATAAGAACATGGAAATCATGGAAGGTAATGCCAAG-3′ (SEQ ID NO: 3).

In some embodiments, the 3′ exon fragment of (C) includes a sequence having at least 85%, 90%, 95%, 97%, 99%, or 100% sequence identity to 5′-GTACCTTTAACTTCCATAAGAACATGGAAATCATGGAAGGTAATGCCAAG-3′ (SEQ ID NO: 3).

In some embodiments, the 3′ exon fragment of (C) includes a sequence having at least 80% sequence identity to 5′-GTACCTTTAACTTCCAAAAGATACATAAAAATCATGGAAGGTAATGCCAAG-3′ (SEQ ID NO: 8).

In some embodiments, the 3′ exon fragment of (C) includes a sequence having at least 85%, 90%, 95%, 97%, 99%, or 100% sequence identity to 5′-GTACCTTTAACTTCCAAAAGATACATAAAAATCATGGAAGGTAATGCCAAG-3′ (SEQ ID NO: 8).

In some embodiments, the 5′ exon fragment of (E) includes a sequence having at least 80% sequence identity to 5′-TTTTTATGTATCTTTTGCGT-3′ (SEQ ID NO: 5).

In some embodiments, the 5′ exon fragment of (E) includes a sequence having at least 85%, 90%, 95%, 97%, 99%, or 100% sequence identity to 5′-TTTTTATGTATOTTTTGCGT-3′ (SEQ ID NO: 5).

In some embodiments, the 3′ exon fragment of (C) includes a sequence having at least 80% sequence identity to 5′-ATGAAGTGAACACGTTATTCAGTTCAAACGGACAGACTCCTTTTGTAACA-3′ (SEQ ID NO: 11).

In some embodiments, the 3′ exon fragment of (C) includes a sequence having at least 85%, 90%, 95%, 97%, 99%, or 100% sequence identity to 5′-ATGAAGTGAACACGTTATTCAGTTCAAACGGACAGACTCCTTTTGTAACA-3′ (SEQ ID NO: 11).

In some embodiments, the 3′ exon fragment of (C) includes a sequence having at least 80% sequence identity to 5′-ATGAAGTGAACACGTTACATAAGCTTGGAATGCAGACTCCTTTTGTAACA-3′ (SEQ ID NO: 16).

In some embodiments, the 3′ exon fragment of (C) includes a sequence having at least 85%, 90%, 95%, 97%, 99%, or 100% sequence identity to 5′-ATGAAGTGAACACGTTACATAAGCTTGGAATGCAGACTCCTTTTGTAACA-3′ (SEQ ID NO: 16).

In some embodiments, the 5′ exon fragment of (E) includes a sequence having at least 80% sequence identity to 5′-TGCATTCCAAGCTTATGAGT-3′ (SEQ ID NO: 13).

In some embodiments, the 5′ exon fragment of (E) includes a sequence having at least 85%, 90%, 95%, 97%, 99%, or 100% sequence identity to 5′-TGCATTCCAAGCTTATGAGT-3′ (SEQ ID NO: 13).

In some embodiments, the 3′ half of Group I catalytic intron fragment of (A) is the 5′ terminus of the linear polynucleotide.

In some embodiments, the 5′ half of Group I catalytic intron fragment of (G) is the 3′ terminus of the linear polyribonucleotide.

In some embodiments, the linear polyribonucleotide does not include a further annealing region. In some embodiments, the linear polyribonucleotide does not include an annealing region 3′ to (A) that includes partial or complete nucleic acid complementarity with an annealing region 5′ to (G). In some embodiments, the polyribonucleotide cargo of (D) includes an expression sequence, a non-coding sequence, or an expression sequence and a non-coding sequence.

In some embodiments, the polyribonucleotide cargo of (D) includes an expression sequence encoding a polypeptide.

In some embodiments, the polyribonucleotide cargo of (D) includes an IRES operably linked to an expression sequence encoding a polypeptide.

In some embodiments, the IRES is located upstream of the expression sequence. In some embodiments, the IRES is located downstream of the expression sequence.

In some embodiments, the polyribonucleotide cargo of (D) includes an expression sequence that encodes a polypeptide that has a biological effect on a subject.

In some embodiments, the linear polyribonucleotide further includes a first spacer region between the 3′ exon fragment of (C) and the polyribonucleotide cargo of (D). The first spacer region may be, e.g., at least 5 (e.g., at least 10, at least 15, at least 20) ribonucleotides in length. In some embodiments, the linear polyribonucleotide further includes a second spacer region between the polyribonucleotide cargo of (D) and the 5′ exon fragment of (E). The second spacer region may be, e.g., at least 5 (e.g., at least 10, at least 15, at least 20) ribonucleotides in length. In some embodiments, each spacer region is at least 5 (e.g., at least 10, at least 15, at least 20) ribonucleotides in length. Each spacer region may be, e.g., from 5 to 500 (e.g., 10, 20, 30, 40, 50, 60, 70, 80, 90, 100, 150, 200, 250, 300, 350, 400, 450, or 500) ribonucleotides in length. The first spacer region, the second spacer region, or the first spacer region and the second spacer region may include a polyA sequence. The first spacer region, the second spacer region, or the first spacer region and the second spacer region may include a polyA-C sequence. The first spacer region, the second spacer region, or the first spacer region and the second spacer region may include a polyA-G sequence. The first spacer region, the second spacer region, or the first spacer region and the second spacer region may include a polyA-T sequence. The first spacer region, the second spacer region, or the first spacer region and the second spacer region may include a random sequence.

In some embodiments, the linear polyribonucleotide is from 50 to 20,000, e.g., 100 to 20,000, e.g., 200 to 20,000, e.g., 300 to 20,000 (e.g., 50, 100, 200, 300, 400, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 5,000, 6,000, 7,000, 8,000, 9,000, 10,000, 11,000, 12,000, 13,000, 14,000, 15,000, 16,000, 17,000, 18,000, 19,000, or 20,000) ribonucleotides in length. In embodiments, the linear polyribonucleotide is, e.g., at least 50, at least 100, at least 200, at least 300, at least 400, at least 500, at least 1,000, at least 2,000, at least 3,000, at least 4,000, or at least 5,000 ribonucleotides in length.

In another aspect, the invention features a DNA vector including an RNA polymerase promoter operably linked to a DNA sequence that encodes the linear polyribonucleotide of any of the embodiments described herein.

In another aspect, the invention features a circular polyribonucleotide (e.g., a covalently closed circular polyribonucleotide) produced from the linear polyribonucleotide or the DNA vector of any of the embodiments described herein.

In some embodiments, the circular polyribonucleotide is from 50 to 20,000, e.g., 100 to 20,000, e.g., 200 to 20,000, e.g., 300 to 20,000 (e.g., 50, 100, 200, 300, 400, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 5,000, 6,000, 7,000, 8,000, 9,000, 10,000, 11,000, 12,000, 13,000, 14,000, 15,000, 16,000, 17,000, 18,000, 19,000, or 20,000) ribonucleotides in length. In embodiments, the circular polyribonucleotide is, e.g., at least 500, at least 1,000, at least 2,000, at least 3,000, at least 4,000, or at least 5,000 ribonucleotides in length.

In some embodiments, the circular polyribonucleotide is produced from a linear polyribonucleotide or vector as described herein.

In another aspect, the invention features a method of expressing a polypeptide in a cell by providing a linear polyribonucleotide, a DNA vector, or a circular polyribonucleotide as described herein to the cell. The method further includes allowing the cellular machinery to express the polypeptide from the polyribonucleotide.

In another aspect, the invention features a method of producing a circular polyribonucleotide as described herein by providing a linear polyribonucleotide as described herein under conditions suitable for self-splicing of the linear polyribonucleotide to produce the circular polyribonucleotide.

### Definitions

To facilitate the understanding of this disclosure, a number of terms are defined below. Terms defined herein have meanings as commonly understood by a person of ordinary skill in the areas relevant to the disclosure. Terms such as “a”, “an,” and “the” are not intended to refer to only a singular entity but include the general class of which a specific example may be used for illustration. The term “or” is used to mean “and/or” unless explicitly indicated to refer to alternatives only or the alternative are mutually exclusive, although the disclosure supports a definition that refers to only alternatives and “and/or”. The terminology herein is used to describe specific embodiments, but their usage is not to be taken as limiting, except as outlined in the claims.

As used herein, any values provided in a range of values include both the upper and lower bounds, and any values contained within the upper and lower bounds.

As used herein, the term “about” refers to a value that is within ±10% of a recited value.

As used herein, the term “carrier” is a compound, composition, reagent, or molecule that facilitates the transport or delivery of a composition (e.g., a circular polyribonucleotide) into a cell by a covalent modification of the circular polyribonucleotide, via a partially or completely encapsulating agent, or a combination thereof. Non-limiting examples of carriers include carbohydrate carriers (e.g., an anhydride-modified phytoglycogen or glycogen-type material), nanoparticles (e.g., a nanoparticle that encapsulates or is covalently linked binds to the circular polyribonucleotide), liposomes, fusosomes, ex vivo differentiated reticulocytes, exosomes, protein carriers (e.g., a protein covalently linked to the circular polyribonucleotide), or cationic carriers (e.g., a cationic lipopolymer or transfection reagent).

As used herein, the terms “circular polyribonucleotide” and “circular RNA” are used interchangeably and mean a polyribonucleotide molecule that has a structure having no free ends (i.e., no free 3′ or 5′ ends), for example a polyribonucleotide molecule that forms a circular or end-less structure through covalent or non-covalent bonds. The circular polyribonucleotide may be, e.g., a covalently closed polyribonucleotide.

As used herein, the term “circularization efficiency” is a measurement of resultant circular polyribonucleotide versus its non-circular starting material.

As used herein, the terms “disease,” “disorder,” and “condition” each refer to a state of sub-optimal health, for example, a state that is or would typically be diagnosed or treated by a medical professional.

The term “derived from” used in the present specification in the context of a nucleic acid, i.e., for a nucleic acid “derived from” (another) nucleic acid, means that the nucleic acid, which is derived from (another) nucleic acid, shares e.g. at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid from which it is derived. The skilled person is aware that sequence identity is typically calculated for the same types of nucleic acids, i.e., for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing the uracils (U) by thymidines (T) throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g., in order to increase RNA stability even further and/or to prolong and/or increase protein production. In the context of amino acid sequences, the term “derived from” means that the amino acid sequence, which is derived from (another) amino acid sequence, shares e.g. at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence from which it is derived.

By “heterologous” is meant to occur in a context other than in the naturally occurring (native) context. A “heterologous” polynucleotide sequence indicates that the polynucleotide sequence is being used in a way other than what is found in that sequence's native genome. For example, a “heterologous promoter” is used to drive transcription of a sequence that is not one that is natively transcribed by that promoter; thus, a “heterologous promoter” sequence is often included in an expression construct by means of recombinant nucleic acid techniques. The term “heterologous” is also used to refer to a given sequence that is placed in a non-naturally occurring relationship to another sequence; for example, a heterologous coding or non-coding nucleotide sequence is commonly inserted into a genome by genomic transformation techniques, resulting in a genetically modified or recombinant genome.

As used herein “increasing fitness” or “promoting fitness” of a subject refers to any favorable alteration in physiology, or of any activity carried out by a subject organism, as a consequence of administration of a peptide or polypeptide described herein, including, but not limited to, any one or more of the following desired effects: (1) increased tolerance of biotic or abiotic stress by about 10%, 20%, 30%, 40%, 50%, 60%, 70%, 80%, 90%, 95%, 99%, 100% or more; (2) increased yield or biomass by about 10%, 20%, 30%, 40%, 50%, 60%, 70%, 80%, 90%, 95%, 99%, 100% or more; (3) modified flowering time by about 10%, 20%, 30%, 40%, 50%, 60%, 70%, 80%, 90%, 95%, 99%, 100% or more; (4) increased resistance to pests or pathogens by about 10%, 20%, 30%, 40%, 50%, 60%, 70%, 80%, 90%, 95%, 99%, 100% or more, (4) increased resistance to herbicides by about 10%, 20%, 30%, 40%, 50%, 60%, 70%, 80%, 90%, 95%, 99%, 100% or more; (5) increasing a population of a subject organism (e.g., an agriculturally important insect) by about 10%, 20%, 30%, 40%, 50%, 60%, 70%, 80%, 90%, 95%, 99%, 100% or more; (6) increasing the reproductive rate of a subject organism (e.g., insect, e.g., bee or silkworm) by about 10%, 20%, 30%, 40%, 50%, 60%, 70%, 80%, 90%, 95%, 99%, 100% or more; (7) increasing the mobility of a subject organism (e.g., insect, e.g., bee or silkworm) by about 10%, 20%, 30%, 40%, 50%, 60%, 70%, 80%, 90%, 95%, 99%, 100% or more; (8) increasing the body weight of a subject organism (e.g., insect, e.g., bee or silkworm) by about 10%, 20%, 30%, 40%, 50%, 60%, 70%, 80%, 90%, 95%, 99%, 100% or more; (9) increasing the metabolic rate or activity of a subject organism (e.g., insect, e.g., bee or silkworm) by about 10%, 20%, 30%, 40%, 50%, 60%, 70%, 80%, 90%, 95%, 99%, 100% or more; (10) increasing pollination (e.g., number of plants pollinated in a given amount of time) by a subject organism (e.g., insect, e.g., bee or silkworm) by about 10%, 20%, 30%, 40%, 50%, 60%, 70%, 80%, 90%, 95%, 99%, 100% or more; (11) increasing production of subject organism (e.g., insect, e.g., bee or silkworm) byproducts (e.g., honey from a honeybee or silk from a silkworm) by about 10%, 20%, 30%, 40%, 50%, 60%, 70%, 80%, 90%, 95%, 99%, 100% or more; (12) increasing nutrient content of the subject organism (e.g., insect) (e.g., protein, fatty acids, or amino acids) by about 10%, 20%, 30%, 40%, 50%, 60%, 70%, 80%, 90%, 95%, 99%, 100% or more; or (13) increasing a subject organism's resistance to pesticides (e.g., a neonicotinoid (e.g., imidacloprid) or an organophosphorus insecticide (e.g., a phosphorothioate, e.g., fenitrothion)) by about 10%, 20%, 30%, 40%, 50%, 60%, 70%, 80%, 90%, 95%, 99%, 100% or more, (14) increasing health or reducing disease of a subject organism such as a human or non-human animal. An increase in host fitness can be determined in comparison to a subject organism to which the modulating agent has not been administered. Conversely, “decreasing fitness” of a subject refers to any unfavorable alteration in physiology, or of any activity carried out by a subject organism, as a consequence of administration of a peptide or polypeptide described herein, including, but not limited to, any one or more of the following intended effects: (1) decreased tolerance of biotic or abiotic stress by about 10%, 20%, 30%, 40%, 50%, 60%, 70%, 80%, 90%, 95%, 99%, 100% or more; (2) decreased yield or biomass by about 10%, 20%, 30%, 40%, 50%, 60%, 70%, 80%, 90%, 95%, 99%, 100% or more; (3) modified flowering time by about 10%, 20%, 30%, 40%, 50%, 60%, 70%, 80%, 90%, 95%, 99%, 100% or more; (4) decreased resistance to pests or pathogens by about 10%, 20%, 30%, 40%, 50%, 60%, 70%, 80%, 90%, 95%, 99%, 100% or more, (4) decreased resistance to herbicides by about 10%, 20%, 30%, 40%, 50%, 60%, 70%, 80%, 90%, 95%, 99%, 100% or more; (5) decreasing a population of a subject organism (e.g., an agriculturally important insect) by about 10%, 20%, 30%, 40%, 50%, 60%, 70%, 80%, 90%, 95%, 99%, 100% or more; (6) decreasing the reproductive rate of a subject organism (e.g., insect, e.g., bee or silkworm) by about 10%, 20%, 30%, 40%, 50%, 60%, 70%, 80%, 90%, 95%, 99%, 100% or more; (7) decreasing the mobility of a subject organism (e.g., insect, e.g., bee or silkworm) by about 10%, 20%, 30%, 40%, 50%, 60%, 70%, 80%, 90%, 95%, 99%, 100% or more; (8) decreasing the body weight of a subject organism (e.g., insect, e.g., bee or silkworm) by about 10%, 20%, 30%, 40%, 50%, 60%, 70%, 80%, 90%, 95%, 99%, 100% or more; (9) decreasing the metabolic rate or activity of a subject organism (e.g., insect, e.g., bee or silkworm) by about 10%, 20%, 30%, 40%, 50%, 60%, 70%, 80%, 90%, 95%, 99%, 100% or more; (10) decreasing pollination (e.g., number of plants pollinated in a given amount of time) by a subject organism (e.g., insect, e.g., bee or silkworm) by about 10%, 20%, 30%, 40%, 50%, 60%, 70%, 80%, 90%, 95%, 99%, 100% or more; (11) decreasing production of subject organism (e.g., insect, e.g., bee or silkworm) byproducts (e.g., honey from a honeybee or silk from a silkworm) by about 10%, 20%, 30%, 40%, 50%, 60%, 70%, 80%, 90%, 95%, 99%, 100% or more; (12) decreasing nutrient content of the subject organism (e.g., insect) (e.g., protein, fatty acids, or amino acids) by about 10%, 20%, 30%, 40%, 50%, 60%, 70%, 80%, 90%, 95%, 99%, 100% or more; or (13) decreasing a subject organism's resistance to pesticides (e.g., a neonicotinoid (e.g., imidacloprid) or an organophosphorus insecticide (e.g., a phosphorothioate, e.g., fenitrothion)) by about 10%, 20%, 30%, 40%, 50%, 60%, 70%, 80%, 90%, 95%, 99%, 100% or more, (14) decreasing health or reducing disease of a subject organism such as a human or non-human animal. A decrease in host fitness can be determined in comparison to a subject organism to which the modulating agent has not been administered. It will be apparent to one of skill in the art that certain changes in the physiology, phenotype, or activity of a subject, e.g., modification of flowering time in a plant, can be considered to increase fitness of the subject or to decrease fitness of the subject, depending on the context (e.g., to adapt to a change in climate or other environmental conditions). For example, a delay in flowering time (e.g., about 10%, 20%, 30%, 40%, 50%, 60%, 70%, 80%, 90%, 95%, 99%, 100% fewer plants in a population flowering at a given calendar date) can be a beneficial adaptation to later or cooler springtimes and thus be considered to increase a plant's fitness; conversely, the same delay in flowering time in the context of earlier or warmer springtimes can be considered to decrease a plant's fitness.

As used herein, the terms “linear RNA” or “linear polyribonucleotide” or “linear polyribonucleotide molecule” are used interchangeably and mean polyribonucleotide molecule having a 5′ and 3′ end. One or both of the 5′ and 3′ ends may be free ends or joined to another moiety. Linear RNA includes RNA that has not undergone circularization (e.g., is pre-circularized) and can be used as a starting material for circularization.

As used herein, the term “modified ribonucleotide” means a nucleotide with at least one modification to the sugar, the nucleobase, or the internucleoside linkage.

As used herein, the term “naked delivery” is a formulation for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a circular polyribonucleotide is a formulation that includes a circular polyribonucleotide without covalent modification and is free from a carrier.

The term “pharmaceutical composition” is intended to also disclose that the circular or linear polyribonucleotide included within a pharmaceutical composition can be used for the treatment of the human or animal body by therapy.

The term “polynucleotide” as used herein means a molecule including one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO3) groups. A nucleotide can include a nucleobase, a five-carbon sugar (either ribose or deoxyribose), and one or more phosphate groups. Ribonucleotides are nucleotides in which the sugar is ribose. Polyribonucleotides or ribonucleic acids, or RNA, can refer to macromolecules that include multiple ribonucleotides that are polymerized via phosphodiester bonds. Deoxyribonucleotides are nucleotides in which the sugar is deoxyribose. As used herein, a polyribonucleotide sequence that recites thymine (T) is understood to represent uracil (U).

As used herein, the term “polyribonucleotide cargo” herein includes any sequence including at least one polyribonucleotide. In embodiments, the polyribonucleotide cargo includes one or multiple expression sequences, wherein each expression sequence encodes a polypeptide. In embodiments, the polyribonucleotide cargo includes one or multiple noncoding sequences, such as a polyribonucleotide having regulatory or catalytic functions. In embodiments, the polyribonucleotide cargo includes a combination of expression and noncoding sequences. In embodiments, the polyribonucleotide cargo includes one or more polyribonucleotide sequence described herein, such as one or multiple regulatory elements, internal ribosomal entry site (IRES) elements, or spacer sequences.

As used interchangeably herein, the terms “polyA” or “polyA sequence” refer to an untranslated, contiguous region of a nucleic acid molecule of at least 5 nucleotides in length and consisting of adenosine residues. In some embodiments, a polyA sequence is at least 10, at least 15, at least 20, at least 30, at least 40, or at least 50 nucleotides in length. In some embodiments, a polyA sequence is located 3′ to (e.g., downstream of) an open reason frame (e.g., an open reading frame encoding a polypeptide), and the polyA sequence is 3′ to a termination element (e.g., a Stop codon) such that the polyA is not translated. In some embodiments, a polyA sequence is located 3′ to a termination element and a 3′ untranslated region.

As used herein, the elements of a nucleic acid are “operably connected” if they are positioned on the vector such that they can be transcribed to form a linear RNA that can then be circularized into a circular RNA using the methods provided herein.

Polydeoxyribonucleotides or deoxyribonucleic acids, or DNA, means macromolecules that include multiple deoxyribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, that include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a polynucleotide is deoxyribonucleic acid (DNA), ribonucleic acid (RNA), or derivatives or variants thereof. In some cases, a polynucleotide is a short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), to name a few, and encompasses both the nucleotide sequence and any structural embodiments thereof, such as single-stranded, double-stranded, triple-stranded, helical, hairpin, etc. In some cases, a polynucleotide molecule is circular. A polynucleotide can have various lengths. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. Embodiments of polynucleotides include isolated and purified DNA/RNA molecules, synthetic DNA/RNA molecules, and synthetic DNA/RNA analogs.

Embodiments of polynucleotides, e.g., polyribonucleotides or polydeoxyribonucleotides, include polynucleotides that contain one or more nucleotide variants, including nonstandard nucleotide(s), non-natural nucleotide(s), nucleotide analog(s) or modified nucleotides. Examples of modified nucleotides include, but are not limited to diaminopurine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. In some cases, nucleotides include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). In embodiments, nucleic acid molecules are modified at the base moiety (e.g., at one or more atoms that typically are available to form a hydrogen bond with a complementary nucleotide or at one or more atoms that are not typically capable of forming a hydrogen bond with a complementary nucleotide), sugar moiety or phosphate backbone. In embodiments, nucleic acid molecules contain amine-modified groups, such as amino allyl 1-dUTP (aa-dUTP) and aminohexylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo or amplification synthesis are described in Betz K, Malyshev D A, Lavergne T, Welte W, Diederichs K, Dwyer T J, Ordoukhanian P, Romesberg F E, Marx A. Nat. Chem. Biol. 2012 July; 8 (7): 612-4, which is herein incorporated by reference for all purposes.

As used herein, “polypeptide” means a polymer of amino acid residues (natural or unnatural) linked together most often by peptide bonds. The term, as used herein, refers to proteins, polypeptides, and peptides of any size, structure, or function. Polypeptides can include gene products, naturally occurring polypeptides, synthetic polypeptides, homologs, orthologs, paralogs, fragments and other equivalents, variants, and analogs of the foregoing. A polypeptide can be a single molecule or a multi-molecular complex such as a dimer, trimer, or tetramer. They can also include single chain or multichain polypeptides such as antibodies or insulin and can be associated or linked. Most commonly disulfide linkages are found in multichain polypeptides. The term polypeptide can also apply to amino acid polymers in which one or more amino acid residues are an artificial chemical analogue of a corresponding naturally occurring amino acid.

As used herein, the term “plant-modifying polypeptide” refers to a polypeptide that can alter the genetic properties (e.g., increase gene expression, decrease gene expression, or otherwise alter the nucleotide sequence of DNA or RNA), epigenetic properties, or biochemical or physiological properties of a plant in a manner that results in a change in the plant's physiology or phenotype, e.g., an increase or a decrease in plant fitness.

As used herein, the term “regulatory element” is a moiety, such as a nucleic acid sequence, that modifies expression of an expression sequence within the circular or linear polyribonucleotide.

As used herein, a “spacer” refers to any contiguous nucleotide sequence (e.g., of one or more nucleotides) that provides distance or flexibility between two adjacent polynucleotide regions.

As used herein, the term “sequence identity” is determined by alignment of two peptide or two nucleotide sequences using a global or local alignment algorithm. Sequences are referred to as “substantially identical” or “essentially similar” when they share at least a certain minimal percentage of sequence identity when optimally aligned (e.g., when aligned by programs such as GAP or BESTFIT using default parameters). GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna, and for proteins the default scoring matrix is Blosum62 (Henikoff & Henikoff, 1992, PNAS 89, 915-919). Sequence alignments and scores for percentage sequence identity are determined, e.g., using computer programs, such as the GCG Wisconsin Package, Version 10.3, available from Accelrys Inc., 9685 Scranton Road, San Diego, CA 92121-3752 USA, or EmbossWin version 2.10.0 (using the program “needle”). Alternatively or additionally, percent identity is determined by searching against databases, e.g., using algorithms such as FASTA, BLAST, etc. Sequence identity refers to the sequence identity over the entire length of the sequence.

As used herein, “structured” with regard to RNA refers to an RNA sequence that is predicted by the RNAFold software or similar predictive tools to form a structure (e.g., a hairpin loop) with itself or other sequences in the same RNA molecule.

As used herein, the term “subject” refers to an organism, such as an animal, plant, or microbe. In embodiments, the subject is a vertebrate animal (e.g., mammal, bird, fish, reptile, or amphibian). In embodiments, the subject is a human. In embodiments, the subject is a non-human mammal. In embodiments, the subject is a non-human mammal such as a non-human primate (e.g., monkeys, apes), ungulate (e.g., cattle, buffalo, bison, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusc. In embodiments, the subject is an invertebrate agricultural pest or an invertebrate that is parasitic on an invertebrate or vertebrate host. In embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses.

As used herein, the term “treat,” or “treating,” refers to a prophylactic or therapeutic treatment of a disease or disorder (e.g., an infectious disease, a cancer, a toxicity, or an allergic reaction) in a subject. The effect of treatment can include reversing, alleviating, reducing severity of, curing, inhibiting the progression of, reducing the likelihood of recurrence of the disease or one or more symptoms or manifestations of the disease or disorder, stabilizing (i.e., not worsening) the state of the disease or disorder, or preventing the spread of the disease or disorder as compared to the state or the condition of the disease or disorder in the absence of the therapeutic treatment. Embodiments include treating plants to control a disease or adverse condition caused by or associated with an invertebrate pest or a microbial (e.g., bacterial, fungal, oomycete, or viral) pathogen. Embodiments include treating a plant to increase the plant's innate defense or immune capability to tolerate pest or pathogen pressure.

As used herein, the term “termination element” is a moiety, such as a nucleic acid sequence, that terminates translation of the expression sequence in the circular or linear polyribonucleotide.

As used herein, the term “translation efficiency” is a rate or amount of protein or peptide production from a ribonucleotide transcript. In some embodiments, translation efficiency can be expressed as amount of protein or peptide produced per given amount of transcript that codes for the protein or peptide, e.g., in a given period of time, e.g., in a given translation system, e.g., an cell-free translation system like rabbit reticulocyte lysate.

As used herein, the term “translation initiation sequence” is a nucleic acid sequence that initiates translation of an expression sequence in the circular or linear polyribonucleotide.

As used herein, the term “therapeutic polypeptide” refers to a polypeptide that when administered to or expressed in a subject provides some therapeutic benefit. In embodiments, a therapeutic polypeptide is used to treat or prevent a disease, disorder, or condition in a subject by administration of the therapeutic peptide to a subject or by expression in a subject of the therapeutic polypeptide. In alternative embodiments, a therapeutic polypeptide is expressed in a cell and the cell is administered to a subject to provide a therapeutic benefit.

As used herein, a “vector” means a piece of DNA, that is synthesized (e.g., using PCR), or that is taken from a virus, plasmid, or cell of a higher organism into which a foreign DNA fragment can be or has been inserted for cloning or expression purposes. In some embodiments, a vector can be stably maintained in an organism. A vector can include, for example, an origin of replication, a selectable marker or reporter gene, such as antibiotic resistance or GFP, or a multiple cloning site (MCS). The term includes linear DNA fragments (e.g., PCR products, linearized plasmid fragments), plasmid vectors, viral vectors, cosmids, bacterial artificial chromosomes (BACs), yeast artificial chromosomes (YACs), and the like. In one embodiment, the vectors provided herein include a multiple cloning site (MCS). In another embodiment, the vectors provided herein do not include an MCS.

### BRIEF DESCRIPTION OF THE DRAWINGS

FIGS. 1A and 1B are schematic drawings showing an exemplary T4 phage permuted intron-exon with an original non-continuous annealing region (FIG. 1A) and an T4 phage 4 permuted intron-exon with an extended continuous annealing region (FIG. 1B).

FIG. 2 is a table showing exemplary modifications for T4 phage nrdB or nrdD permuted intron-exon with a non-continuous annealing region (SEQ ID NOs: 52-55). Bolding identifies the modified nucleotides, underlining identifies added or deleted nucleotides; bold line identifies new complementary regions.

FIG. 3 is a graph showing the circularization efficiency of T4 phage permuted intron-exon with an original non-continuous annealing region (nrdB1 or nrdD1) and an T4 phage 4 permuted intron-exon with an extended continuous annealing region (nrdB2 or nrdD2).

FIG. 4 is a graph showing the circularization efficiency of T4 phage permuted intron-exon with an original non-continuous annealing region (nrdB1 or nrdD1), a T4 phage permuted intron-exon with an extended continuous annealing region (nrdB2 or nrdD2), and an Anabaena permuted intron-exon with extended annealing region (Ana2-1 or Ana2-2).

FIG. 5 is a graph showing immune response as measured by IFN-β (pg/mL) in A549 cells with nrdB1, nrdB2, nrdD1, Ana2-1, and Ana2-2 constructs.

FIG. 6 is a graph showing immune response as measured by IP-10 (pg/mL) in macrophages with the nrdB1, nrdD1, Ana2-1, and Ana2-2 constructs.

### DETAILED DESCRIPTION

The present invention features compositions and methods for producing a circular polyribonucleotide (circular RNA). Circular polyribonucleotides described herein are particularly useful for delivering a polynucleotide cargo (e.g., encoding a gene or protein) to a target cell.

A circular polyribonucleotide may be produced from a linear polyribonucleotide in which the ends are self-spliced together, thereby forming the circular polyribonucleotide. The linear RNA molecules described herein include, from 5′ to 3′, (A) a 3′ half of Group I catalytic intron fragment from a T4 phage nrdB gene or nrdD gene; (B) a 3′ splice site; (C) a 3′ exon fragment; (D) a polyribonucleotide cargo; (E) a 5′ exon fragment; (F) a 5′ splice site; and (G) a 5′ half of Group I catalytic intron fragment from a T4 phage nrdB gene or nrdD gene. The polyribonucleotide includes a first annealing region that has from 2 to 50, e.g., from 8 to 50 ribonucleotides and is present within (A) the 3′ half of Group I catalytic intron fragment from the T4 phage nrdB gene or nrdD gene; (B) the 3′ splice site; or (C) the 3′ exon fragment. The polyribonucleotide also includes a second annealing region that has from 2 to 50, e.g., from 8 to 50 ribonucleotides and is present within (E) the 5′ exon fragment; (F) the 5′ splice site; or (G) the 5′ half of Group I catalytic intron fragment from the T4 phage nrdB gene or nrdD gene. The first annealing region has from 80% to 100% complementarity with the second annealing region or has from zero to 10 mismatched base pairs. These features allow the first annealing region to hybridize to the second annealing region, thus bringing the splice sites near the 5′ and 3′ ends of the linear polyribonucleotide into close proximity. Once the splice sites are nearby, the polyribonucleotide is able to self-splice the 3′ and 5′ splice sites, thus forming the circular polyribonucleotide.

By including the first annealing region within, for example, (A) the 3′ half of Group I catalytic intron fragment; (B) the 3′ splice site; or (C) the 3′ exon fragment, and the second annealing region within, for example, (E) the 5′ exon fragment; (F) the 5′ splice site; or (G) the 5′ half of Group I catalytic intron fragment, the linear molecule exhibits increased circularization efficiency and splicing fidelity as compared to other polyribonucleotide constructs that lack these features. Furthermore, by using an autocatalytic self-splicing intron from a T4 phage nrdB or nrdD gene, the linear molecule does not need to be treated with an exogenous enzyme, such as a ligase, to produce the circular polyribonucleotide. This is particularly advantageous for producing a circular product in a single pot reaction. The molecules, methods of producing, and uses thereof are described in more detail below.

### Polynucleotides

The disclosure features circular polyribonucleotide compositions and methods of making circular polyribonucleotides. In some embodiments, a circular polyribonucleotide is produced from a linear polyribonucleotide (e.g., by self-splicing compatible ends of the linear polyribonucleotide). In some embodiments, a linear polyribonucleotide is transcribed from a deoxyribonucleotide template (e.g., a vector, a linearized vector, or a cDNA). Accordingly, the disclosure features deoxyribonucleotides, linear polyribonucleotides, and circular polyribonucleotides and compositions thereof useful in the production of circular polyribonucleotides.

### Template Deoxyribonucleotides

The present invention features a template deoxyribonucleotide for making circular RNA. The deoxyribonucleotide includes the following, operably linked in a 5′-to-3′ orientation: (A) a 3′ half of Group I catalytic intron fragment from a T4 phage nrdB gene or nrdD gene; (B) a 3′ splice site; (C) a 3′ exon fragment; (D) a polyribonucleotide cargo; (E) a 5′ exon fragment; (F) a 5′ splice site; and (G) a 5′ half of Group I catalytic intron fragment from a T4 phage nrdB gene or nrdD gene. In embodiments, the deoxyribonucleotide includes further elements, e.g., outside of or between any of elements (A), (B), (C), (D), (E), (F), or (G). In embodiments, any of the elements (A), (B), (C), (D), (E), (F), or (G) is separated from each other by a spacer sequence, as described herein.

In embodiments, the deoxyribonucleotide is, for example, a circular DNA vector, a linearized DNA vector, or a linear DNA (e.g., a cDNA, e.g., produced from a DNA vector).

In some embodiments, the deoxyribonucleotide further includes an RNA polymerase promoter operably linked to a sequence encoding a linear RNA described herein. In embodiments, the RNA polymerase promoter is heterologous to the sequence encoding the linear RNA. In some embodiments, the RNA polymerase promoter is a T7 promoter, a T6 promoter, a T4 promoter, a T3 promoter, an SP6 virus promoter, or an SP3 promoter.

In some embodiments, the deoxyribonucleotide includes a multiple-cloning site (MCS).

In some embodiments, the deoxyribonucleotide is used to produce circular RNA with the size range of about 100 to about 20,000 nucleotides. In some embodiments, the circular RNA is at least 100, 500, 550, 600, 650, 700, 750, 800, 850, 900, 950, 1,000, 1,100, 1,200, 1,300, 1,400, 1,500, 1,600 1,700, 1,800, 1,900, 2,000, 2,500, 3,000, 3,500, 4,000, 4,500 or 5,000 nucleotides in size. In some embodiments, the circular RNA is no more than 20,000, 15,000 10,000, 9,000, 8,000, 7,000, 6,000, 5,000 or 4,000 nucleotides in size.

### Linear Polyribonucleotides

The present invention also features linear polyribonucleotides including the following, operably linked in a 5′-to-3′ orientation: (A) a 3′ half of Group I catalytic intron fragment from a T4 phage nrdB gene or nrdD gene; (B) a 3′ splice site; (C) a 3′ exon fragment; (D) a polyribonucleotide cargo; (E) a 5′ exon fragment; (F) a 5′ splice site; and (G) a 5′ half of Group I catalytic intron fragment from a T4 phage nrdB gene or nrdD gene. In embodiments, the linear polyribonucleotide includes further elements, e.g., outside of or between any of elements (A), (B), (C), (D), (E), (F), or (G). For example, any of elements (A), (B), (C), (D), (E), (F), or (G) may be separated by a spacer sequence, as described herein.

In certain embodiments, provided herein is a method of generating linear RNA by performing transcription in a cell-free system (e.g., in vitro transcription) using a deoxyribonucleotide (e.g., a vector, linearized vector, or cDNA) provided herein as a template (e.g., a vector, linearized vector, or cDNA provided herein with an RNA polymerase promoter positioned upstream of the region that codes for the linear RNA).

In embodiments, a deoxyribonucleotide template is transcribed to a produce a linear RNA containing the components described herein. Upon expression, the linear polyribonucleotide produces a splicing-compatible polyribonucleotide, which may be self-spliced in order to produce a circular polyribonucleotide.

In some embodiments, the linear polyribonucleotide is from 50 to 20,000, 100 to 20,000, 200 to 20,000, 300 to 20,000 (e.g., 50, 100, 200, 300, 400, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 5,000, 6,000, 7,000, 8,000, 9,000, 10,000, 11,000, 12,000, 13,000, 14,000, 15,000, 16,000, 17,000, 18,000, 19,000, or 20,000) ribonucleotides in length. In embodiments, the linear polyribonucleotide is, e.g., at least 500, at least 1,000, at least 2,000, at least 3,000, at least 4,000, or at least 5,000 ribonucleotides in length.

### Circular Polyribonucleotides

In some embodiments, the invention features a circular polyribonucleotide (e.g., a covalently closed circular polyribonucleotide). In embodiments, the circular polyribonucleotide includes a splice junction joining a 5′ exon fragment and a 3′ exon fragment. In embodiments, the 3′ exon fragment includes the first annealing region having from 2 to 50, e.g., from 8 to 50 (e.g., from 10 to 30, 10 to 20, or 10 to 15, e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) ribonucleotides, and the 5′ exon fragment includes the second annealing region having from 2 to 50, e.g., from 8 to 50 (e.g., from 10 to 30, 10 to 20, or 10 to 15, e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) ribonucleotides. In embodiments, the first annealing region and the second annealing region include from 80% to 100% (e.g., 80%, 85%, 90%, 95%, 97%, 99%, or 100%) complementarity. In embodiments, the first annealing region and the second annealing region include from zero to 10 (e.g., 0, 1, 2, 3, 4, 5, 6, 7, 8, 9, or 10) mismatched base pairs.

In embodiments, the circular polynucleotide further includes a polyribonucleotide cargo. In embodiments, the polyribonucleotide cargo includes an expression (or coding) sequence, a non-coding sequence, or a combination of an expression (coding) sequence and a non-coding sequence. In embodiments, the polyribonucleotide cargo includes an expression (coding) sequence encoding a polypeptide. In embodiments, the polyribonucleotide includes an IRES operably linked to an expression sequence encoding a polypeptide. In some embodiments, the IRES is located upstream of the expression sequence. In some embodiments, the IRES is located downstream of the expression sequence. In some embodiments, the circular polyribonucleotide further includes a spacer region between the IRES and the 3′ exon fragment or the 5′ exon fragment. The spacer region may be, e.g., at least 5 (e.g., at least 10, at least 15, at least 20) ribonucleotides in length ribonucleotides in length. The spacer region may be, e.g., from 5 to 500 (e.g., 10, 20, 30, 40, 50, 60, 70, 80, 90, 100, 150, 200, 250, 300, 350, 400, 450, or 500) ribonucleotides. In some embodiments, the spacer region includes a polyA sequence. In some embodiments, the spacer region includes a polyA-C sequence. In some embodiments, the spacer region includes a polyA-G sequence. In some embodiments, the spacer region includes a polyA-T sequence. In some embodiments, the spacer region includes a random sequence. In some embodiments, the first annealing region and the second annealing region are joined, thereby forming a circular polyribonucleotide.

In some embodiments, the circular RNA is a produced by a deoxyribonucleotide template or a linear RNA described herein. In some embodiments, the circular RNA is produced by any of the methods described herein.

In some embodiments, the circular polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

In some embodiments, the circular polyribonucleotide is of a sufficient size to accommodate a binding site for a ribosome. In some embodiments, the size of a circular polyribonucleotide is a length sufficient to encode useful polypeptides, e.g., at least 20,000 nucleotides, at least 15,000 nucleotides, at least 10,000 nucleotides, at least 7,500 nucleotides, at least 5,000 nucleotides, at least 4,000 nucleotides, at least 3,000 nucleotides, at least 2,000 nucleotides, at least 1,000 nucleotides, at least 500 nucleotides, at least 1400 nucleotides, at least 300 nucleotides, at least 200 nucleotides, or at least 100 nucleotides may be produced.

In some embodiments, the circular polyribonucleotide includes one or more elements described elsewhere herein. In some embodiments, the elements are separated from one another by a spacer sequence. In some embodiments, the elements are separated from one another by 1 ribonucleotide, 2 nucleotides, about 5 nucleotides, about 10 nucleotides, about 15 nucleotides, about 20 nucleotides, about 30 nucleotides, about 40 nucleotides, about 50 nucleotides, about 60 nucleotides, about 80 nucleotides, about 100 nucleotides, about 150 nucleotides, about 200 nucleotides, about 250 nucleotides, about 300 nucleotides, about 400 nucleotides, about 500 nucleotides, about 600 nucleotides, about 700 nucleotides, about 800 nucleotides, about 900 nucleotides, about 1000 nucleotides, up to about 1 kb, at least about 1000 nucleotides, or any amount of nucleotides therebetween. In some embodiments, one or more elements are contiguous with one another, e.g., lacking a spacer element.

In some embodiments, the circular polyribonucleotide includes one or more repetitive elements described elsewhere herein. In some embodiments, the circular polyribonucleotide includes one or more modifications described elsewhere herein. In one embodiment, the circular RNA contains at least one nucleoside modification. In one embodiment, up to 100% of the nucleosides of the circular RNA are modified. In one embodiment, at least one nucleoside modification is a uridine modification or an adenosine modification.

As a result of its circularization, the circular polyribonucleotide may include certain characteristics that distinguish it from linear RNA. For example, the circular polyribonucleotide is less susceptible to degradation by exonuclease as compared to linear RNA. As such, the circular polyribonucleotide is more stable than a linear RNA, especially when incubated in the presence of an exonuclease. The increased stability of the circular polyribonucleotide compared with linear RNA makes circular polyribonucleotide more useful as a cell transforming reagent to produce polypeptides and can be stored more easily and for longer than linear RNA. The stability of the circular polyribonucleotide treated with exonuclease can be tested using methods standard in art which determine whether RNA degradation has occurred (e.g., by gel electrophoresis). Moreover, unlike linear RNA, the circular polyribonucleotide is less susceptible to dephosphorylation when the circular polyribonucleotide is incubated with phosphatase, such as calf intestine phosphatase.

### Annealing Regions

Polynucleotide compositions described herein may include two or more annealing regions, e.g., two or more annealing regions described herein. An annealing region, or pair of annealing regions, are those that contain a portion with a high degree of complementarity that promotes hybridization under suitable conditions.

An annealing region includes at least a region of complementary as described herein. The high degree of complementarity of the complementary region promotes the association of annealing region pairs. When a first annealing region (e.g., a 5′ annealing region) is located at or near the 5′ end of a linear RNA and a second annealing region (e.g., a 3′ annealing region) is located at or near the 3′ end of a linear RNA, association of the annealing regions brings the 5′ and 3′ and the corresponding intron fragments into proximity. In some embodiments, this favor circularization of the linear RNA by splicing of the 3′ and 5′ splice sites. In some embodiments, the annealing regions described herein strengthen naturally occurring annealing regions, e.g., to promote self-splicing.

An annealing region may be altered by introducing one or more (e.g., 1, 2, 3, 4, 5, 6, 7, 8, 9, 10, 11, 12, 13, 14, 15, 16, 17, 18, 19, 20, or more) mutations into the polyribonucleotide sequence. For example, an annealing region may be extended by introducing one or more point mutations into a first annealing region and/or a second annealing region to increase the length of complementarity between the first and second annealing regions. The annealing region may also be altered by inserting one or more (e.g., 1, 2, 3, 4, 5, 6, 7, 8, 9, 10, 11, 12, 13, 14, 15, 16, 17, 18, 19, 20, or more) nucleotides into the polyribonucleotide. In embodiments, an annealing region is extended by inserting one or more nucleotides into a first annealing region and/or a second annealing region to increase the length of complementarity between the first and second annealing regions. In embodiments, the annealing region is extended by introducing one or more point mutations into a first annealing and/or a second region and inserting one or more nucleotides into the first annealing and/or the second annealing region to increase the length of complementarity. Altering the annealing region may alter the secondary structure of the polyribonucleotide by favoring a bulge or mismatched region with the original sequence to preferentially form a stem or stem loop structure with the altered sequence.

The polyribonucleotide includes a first annealing region that has from 2 to 50, 5 to 50, 6 to 50, 7 to 50, or 8 to 50 (e.g., from 10 to 30, 10 to 20, or 10 to 15, e.g., at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) ribonucleotides and is present within (A) the 3′ half of Group I catalytic intron fragment from a T4 phage nrdB gene or nrdD gene; (B) the 3′ splice site; or (C) the 3′ exon fragment. The polyribonucleotide also includes a second annealing region that has from 2 to 50, 5 to 50, 6 to 50, 7 to 50, or 8 to 50 (e.g., from 10 to 30, 10 to 20, or 10 to 15, e.g., at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) ribonucleotides and is present within (E) the 5′ exon fragment; (F) the 5′ splice site; or (G) the 5′ half of Group I catalytic intron fragment from a T4 phage nrdB gene or nrdD gene. The first annealing region has from 80% to 100% (e.g., 85% to 100%, e.g., 90% to 100%, e.g., 80%, 85%, 90%, 95%, 97%, 99%, or 100%) complementarity with the second annealing region or has from zero to 10 e.g., (0, 1, 2, 3, 4, 5, 6, 7, 8, 9, or 10) mismatched base pairs.

In some embodiments, the first annealing region and the second annealing region are 100% complementary.

In some embodiments, (A) or (C) includes the first annealing region and (E) or (G) includes the second annealing region.

In some embodiments, the 3′ exon fragment of (C) includes the first annealing region and the 5′ exon fragment of (E) includes the second annealing region.

In some embodiments, the 3′ half of Group I catalytic intron fragment of (A) includes the first annealing region and the 5′ exon fragment of (E) includes the second annealing region.

In some embodiments, the 3′ exon fragment of (C) includes the first annealing region and the 5′ half of Group I catalytic intron fragment includes the second annealing region.

In some embodiments, first annealing region and the second annealing region include zero or one mismatched base pair.

In embodiments, an annealing region further includes a non-complementary region as described below. A non-complementary region may be added to the complementary region to allow for the ends of the RNA to remain flexible, unstructured, or less structured than the complementarity region.

In some embodiments, each annealing region includes 2 to 100, 5 to 100, or 6 to 100 ribonucleotides (e.g., 6 to 80, 6 to 50, 6 to 30, 6 to 20, 10 to 100, 10 to 80, 10 to 50, or 10 to 30 ribonucleotides). In some embodiments, a 5′ annealing region includes 2 to 100, 5 to 100, 6 to 100 ribonucleotides (e.g., 6 to 80, 6 to 50, 6 to 30, 6 to 20, 10 to 100, 10 to 80, 10 to 50, or 10 to 30 ribonucleotides). In some embodiments, a 3′ annealing region includes 6 to 100 ribonucleotides (e.g., 6 to 80, 6 to 50, 6 to 30, 6 to 20, 10 to 100, 10 to 80, 10 to 50, or 10 to 30 ribonucleotides).

In some embodiments, the polyribonucleotide does not include an annealing region 3′ to (A) that includes partial or complete nucleic acid complementarity with an annealing region 5′ to (G).

In some embodiments, the polyribonucleotide does not include a further annealing region, e.g., in addition to the first annealing region and second annealing region.

### Complementary Regions

A complementary region is a region that favors association with a corresponding complementary region, under suitable conditions. For example, a pair of complementary regions may share a high degree of sequence complementarity (e.g., a first complementary region is the reverse complement of a second complementary region, at least in part). When two complementary regions associate (e.g., hybridize), they may form a highly structured secondary structure, such as a stem or stem loop.

In some embodiments, the polyribonucleotide includes a 5′ complementary region and a 3′ complementary region. In some embodiments, the 5′ complementary region has from 2 to 50, e.g., 5 to 50 ribonucleotides (e.g., 5-40, 5-30, 5-20, 5-10, 10-50, 10-40, 10-30, 10-20, or 20-50, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 ribonucleotides). In some embodiments, the 3′ complementary region has from 2 to 50, e.g., 5 to 50 ribonucleotides (e.g., 5-40, 5-30, 5-20, 5-10, 10-50, 10-40, 10-30, 10-20, or 20-50, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 ribonucleotides).

In some embodiments, the 5′ complementary region and the 3′ complementary region have from 50% to 100% sequence complementarity (e.g., from 60%-100%, 70%-100%, 80%-100%, 90%-100%, or 100%, e.g., 80%, 85%, 90%, 95%, 97%, 99%, or 100% sequence complementarity).

In some embodiments, the 5′ complementary region and the 3′ complementary region have a free energy of binding of less than-5 kcal/mol (e.g., less than-10 kcal/mol, less than-20 kcal/mol, or less than −30 kcal/mol).

In some embodiments, the 5′ complementary region and the 3′ complementary region have a Tm of binding of at least 10° C., at least 15° C., at least 20° C., at least 30° C., at least 40° C., at least 50° C., at least 60° C., at least 70° C., at least 80° C., or at least 90° C.

In some embodiments, the 5′ complementary region and the 3′ complementary region include at least one but no more than 10 mismatches, e.g., 10, 9, 8, 7, 6, 5, 4, 3, or 2 mismatches, or 1 mismatch (i.e., when the 5′ complementary region and the 3′ complementary region hybridize to each other). A mismatch can be, e.g., a nucleotide in the 5′ complementary region and a nucleotide in the 3′ complementary region that are opposite each other (i.e., when the 5′ complementary region and the 3′ complementary region are hybridized) but that do not form a Watson-Crick base-pair. A mismatch can be, e.g., an unpaired nucleotide that forms a kink or bulge in either the 5′ complementary region or the 3′ complementary region. In some embodiments, the 5′ complementary region and the 3′ complementary region do not include any mismatches.

### Non-Complementary Regions

A non-complementary region is a region that disfavors association with a corresponding non-complementary region, under suitable conditions. For example, a pair of non-complementary regions may share a low degree of sequence complementarity (e.g., a first non-complementary region is not a reverse complement of a second non-complementary region). When two non-complementary regions are in proximity, they do not form a highly structured secondary structure, such as a stem or stem loop.

In some embodiments, the polyribonucleotide includes a 5′ non-complementary region and a 3′ non-complementary region. In some embodiments, the 5′ non-complementary region has from 5 to 50 ribonucleotides (e.g., 5-40, 5-30, 5-20, 5-10, 10-50, 10-40, 10-30, 10-20, or 20-50 ribonucleotides). In some embodiments, the 3′ non-complementary region has from 5 to 50 ribonucleotides (e.g., 5-40, 5-30, 5-20, 5-10, 10-50, 10-40, 10-30, 10-20, or 20-50 ribonucleotides).

In some embodiments the 5′ non-complementary region is located 5′ to the 5′ complementary region (e.g., between the 5′ catalytic intron fragment and the 5′ complementary region). In some embodiments, the 3′ non-complementary region is located 3′ to the 3′ complementary region (e.g., between the 3′ complementary region and the 3′ catalytic intron fragment).

In some embodiments, the 5′ non-complementary region and the 3′ non-complementary region have from 0% to 50% sequence complementarity (e.g., from 0%-40%, 0%-30%, 0%-20%, 0%-10%, or 0% sequence complementarity).

In some embodiments, the 5′ non-complementary region and the 3′ non-complementary region have a free energy of binding of greater than-5 kcal/mol.

In some embodiments, the 5′ complementary region and the 3′ complementary region have a Tm of binding of less than 10° C.

In some embodiments, the 5′ non-complementary region and the 3′ non-complementary region include at least 1, 2, 3, 4, 5, 6, 7, 8, 9, or 10 mismatches.

### Catalytic Introns

The polyribonucleotides described herein include catalytic intron fragments, such as (A) a 3′ half of Group I catalytic intron fragment from a T4 phage nrdB gene or nrdD gene and (G) a 5′ half of Group I catalytic intron fragment from a T4 phage nrdB gene or nrdD gene. The first and second annealing regions may be positioned within the catalytic intron fragments. Group I catalytic introns are self-splicing ribozymes that catalyze their own excision from mRNA, tRNA, and rRNA precursors via two-metal ion phosphoryl transfer mechanism. Importantly, the RNA itself self-catalyzes the intron removal without the requirement of an exogenous enzyme, such as a ligase.

In some embodiments, the 3′ half of Group I catalytic intron fragment of (A) and the 5′ Group I catalytic intron fragment of (G) are from a T4 phage td gene. The 3′ exon fragment of (C) may include the first annealing region and the 5′ half of Group I catalytic intron fragment of (G) may include the second annealing region. The first annealing region T4 phage td gene may include, e.g., from 2 to 16, e.g., 10 to 16 (e.g., 2, 3, 4, 5, 6, 7, 8, 9, 10, 11, 12, 13, 14, 15, or 16) ribonucleotides, and the second annealing region may include, e.g., from 2 to 16, e.g., 10 to 16 (e.g., 2, 3, 4, 5, 6, 7, 8, 9, 10, 11, 12, 13, 14, 15, or 16) ribonucleotides.

In some embodiments, the 3′ half of Group I catalytic intron fragment of (A) is the 5′ terminus of the linear polynucleotide.

In some embodiments, the 5′ half of Group I catalytic intron fragment of (G) is the 3′ terminus of the linear polyribonucleotide.

In some embodiments, the 3′ half of Group I catalytic intron fragment of (A) is from the T4 phage nrdB gene.

In some embodiments, the 3′ half of Group I catalytic intron fragment of (A) includes a sequence having at least 80% sequence identity to 5′-TTGCAAAACAAGGTTCAACGACTAGTCTTCGGACGTAGGGTCAAGCGACTCGAAATGGGGAGAATC CCTCCGGGATTGTGATATAGTCTGGACTGCATGGTAACATGCAGCAGTTCATAAGAGAACGGGTTGA GAATTAGCGAGCTCAATCGAACATACG-3′ (SEQ ID NO: 2).

In some embodiments, the 3′ half of Group I catalytic intron fragment of (A) includes a sequence having at least 85%, 90%, 95%, 97%, 99%, or 100% sequence identity to 5′-TTGCAAAACAAGGTTCAACGACTAGTCTTCGGACGTAGGGTCAAGCGACTCGAAATGGGGAGAATC CCTCCGGGATTGTGATATAGTCTGGACTGCATGGTAACATGCAGCAGTTCATAAGAGAACGGGTTGA GAATTAGCGAGCTCAATCGAACATACG-3′ (SEQ ID NO: 2).

In some embodiments, the 5′ half of Group I catalytic intron fragment of (G) is from the T4 phage nrdB gene. In some embodiments, the 3′ half of Group I catalytic intron fragment of (A) is from the T4 phage nrdB gene and the 5′ half of Group I catalytic intron fragment of (G) is from the T4 phage nrdB gene.

In some embodiments, the 5′ half of Group I catalytic intron of (G) includes a sequence having at least 80% sequence identity to 5′-AAAATGCGCCTTTAAACGGTAACGTTTATCGAAAACTCCTTTAATTGCTGGAAAGTCCTTTATGGAAA ACTAGCAGCCAAGGTTTTGCTT-3′ (SEQ ID NO: 6).

In some embodiments, the 5′ half of Group I catalytic intron of (G) includes a sequence having at least 85%, 90%, 95%, 97%, 99%, or 100% sequence identity to 5′-AAAATGCGCCTTTAAACGGTAACGTTTATCGAAAACTCCTTTAATTGCTGGAAAGTCCTTTATGGAAA ACTAGCAGCCAAGGTTTTGCTT-3′ (SEQ ID NO: 6).

In some embodiments, the 3′ half of Group I catalytic intron fragment of (A) is from the T4 phage nrdD gene.

In some embodiments, the 3′ half of Group I catalytic intron fragment of (A) includes a sequence having at least 80% sequence identity to 5′-CAGTAGCTGTAAATGCCCAACGACTATCCCTGATGAATGTAAGGGAGTAGGGTCAAGCGACCCGAA

ACGGCAGACAACTCTAAGAGTTGAAGATATAGTCTGAACTGCATGGTGACATGCAGCTGTTTATCCT CGTATAAATATGAATACGAGGTGAAACGATGAAATGAATTACATTGTTTCATATAAACGGGTAGAGAA GTAGCGAACTCTACTGAACACATTG-3′ (SEQ ID NO: 10).

In some embodiments, the 3′ half of Group I catalytic intron fragment of (A) includes a sequence having at least 85%, 90%, 95%, 97%, 99%, or 100% sequence identity to 5′-CAGTAGCTGTAAATGCCCAACGACTATCCCTGATGAATGTAAGGGAGTAGGGTCAAGCGACCCGAA ACGGCAGACAACTCTAAGAGTTGAAGATATAGTCTGAACTGCATGGTGACATGCAGCTGTTTATCCT CGTATAAATATGAATACGAGGTGAAACGATGAAATGAATTACATTGTTTCATATAAACGGGTAGAGAA GTAGCGAACTCTACTGAACACATTG-3′ (SEQ ID NO: 10).

In some embodiments, the 5′ half of Group I catalytic intron fragment of (G) is from the T4 phage nrdD gene. In some embodiments, the 3′ half of Group I catalytic intron fragment of (A) is from the T4 phage nrdD gene and the 5′ half of Group I catalytic intron fragment of (G) is from the T4 phage nrdD gene.

In some embodiments, the 5′ half of Group I catalytic intron of (G) includes a sequence having at least 80% sequence identity to 5′-TAACGTAAGTCAAGCTCATGTAAAATCTGCCTAAAACGGGAAACTCTCACTGAGACAATCCGTTGCTA AATCAG-3′ (SEQ ID NO: 14).

In some embodiments, the 5′ half of Group I catalytic intron of (G) includes a sequence having at least 85%, 90%, 95%, 97%, 99%, or 100% sequence identity to 5′-TAACGTAAGTCAAGCTCATGTAAAATCTGCCTAAAACGGGAAACTCTCACTGAGACAATCCGTTGCTA AATCAG-3′ (SEQ ID NO: 14).

In some embodiments, the 3′ exon fragment of (C) includes a sequence having at least 80% sequence identity to 5′-GTACCTTTAACTTCCATAAGAACATGGAAATCATGGAAGGTAATGCCAAG-3′ (SEQ ID NO: 3).

In some embodiments, the 3′ exon fragment of (C) includes a sequence having at least 85%, 90%, 95%, 97%, 99%, or 100% sequence identity to 5′-GTACCTTTAACTTCCATAAGAACATGGAAATCATGGAAGGTAATGCCAAG-3′ (SEQ ID NO: 3).

In some embodiments, the 3′ exon fragment of (C) includes a sequence having at least 80% sequence identity to 5′-GTACCTTTAACTTCCAAAAGATACATAAAAATCATGGAAGGTAATGCCAAG-3′ (SEQ ID NO: 8).

In some embodiments, the 3′ exon fragment of (C) includes a sequence having at least 85%, 90%, 95%, 97%, 99%, or 100% sequence identity to 5′-GTACCTTTAACTTCCAAAAGATACATAAAAATCATGGAAGGTAATGCCAAG-3′ (SEQ ID NO: 8).

In some embodiments, the 5′ exon fragment of (E) includes a sequence having at least 80% sequence identity to 5′-TTTTTATGTATOTTTTGCGT-3′ (SEQ ID NO: 5).

In some embodiments, the 5′ exon fragment of (E) includes a sequence having at least 85%, 90%, 95%, 97%, 99%, or 100% sequence identity to 5′-TTTTTATGTATOTTTTGCGT-3′ (SEQ ID NO: 5). In some embodiments, the 3′ exon fragment of (C) includes a sequence having at least 80% sequence identity to 5′-ATGAAGTGAACACGTTATTCAGTTCAAACGGACAGACTCCTTTTGTAACA-3′ (SEQ ID NO: 11).

In some embodiments, the 3′ exon fragment of (C) includes a sequence having at least 85%, 90%, 95%, 97%, 99%, or 100% sequence identity to 5′-ATGAAGTGAACACGTTATTCAGTTCAAACGGACAGACTCCTTTTGTAACA-3′ (SEQ ID NO: 11).

In some embodiments, the 3′ exon fragment of (C) includes a sequence having at least 80% sequence identity to 5′-ATGAAGTGAACACGTTACATAAGCTTGGAATGCAGACTCCTTTTGTAACA-3′ (SEQ ID NO: 16).

In some embodiments, the 3′ exon fragment of (C) includes a sequence having at least 85%, 90%, 95%, 97%, 99%, or 100% sequence identity to 5′-ATGAAGTGAACACGTTACATAAGCTTGGAATGCAGACTCCTTTTGTAACA-3′ (SEQ ID NO: 16).

In some embodiments, the 5′ exon fragment of (E) includes a sequence having at least 80% sequence identity to 5′-TGCATTCCAAGCTTATGAGT-3′ (SEQ ID NO: 13).

In some embodiments, the 5′ exon fragment of (E) includes a sequence having at least 85%, 90%, 95%, 97%, 99%, or 100% sequence identity to 5′-TGCATTCCAAGCTTATGAGT-3′ (SEQ ID NO: 13)

### Splice Sites

The polyribonucleotides described herein include splice sites, such as (B) a 3′ splice site; and (F) a 5′ splice site. In embodiments, the splice site is from a T4 phage nrdB gene or nrdD gene.

In some embodiments, the 3′ splice site (e.g., between the 3′ half of Group I catalytic intron fragment and the 3′ exon fragment has the sequence of ATACG↓GTACC (SEQ ID NO: 40) where the arrow denotes the cut site. In some embodiments, the 5′ splice site (e.g., between the 5′ exon fragment and the 5′ half of Group I catalytic intron fragment has the sequence of TGCGT↓AAAAT (SEQ ID NO: 41) where the arrow denotes the cut site.

In some embodiments, the 3′ splice site (e.g., between the 3′ half of Group I catalytic intron fragment and the 3′ exon fragment has the sequence CATTG↓ATGAA (SEQ ID NO: 42) where the arrow denotes the cut site. In some embodiments, the 5′ splice site (e.g., between the 5′ exon fragment and the 5′ half of Group I catalytic intron fragment has the sequence of TGAGT↓TAACG (SEQ ID NO: 43) where the arrow denotes the cut site.

### Exon Fragments

The polyribonucleotides described herein include an exon fragment, such as (C) a 3′ exon fragment; and (E) a 5′ exon fragment.

In some embodiments, the 3′ exon fragment of (C) includes a sequence having at least 80% sequence identity to 5′-GTACCTTTAACTTCCATAAGAACATGGAAATCATGGAAGGTAATGCCAAG-3′ (SEQ ID NO: 3).

In some embodiments, the 3′ exon fragment of (C) includes a sequence having at least 85%, 90%, 95%, 97%, 99%, or 100% sequence identity to 5′-GTACCTTTAACTTCCATAAGAACATGGAAATCATGGAAGGTAATGCCAAG-3′ (SEQ ID NO: 3).

In some embodiments, the 3′ exon fragment of (C) includes a sequence having at least 80% sequence identity to 5′-GTACCTTTAACTTCCAAAAGATACATAAAAATCATGGAAGGTAATGCCAAG-3′ (SEQ ID NO: 8).

In some embodiments, the 3′ exon fragment of (C) includes a sequence having at least 85%, 90%, 95%, 97%, 99%, or 100% sequence identity to 5′-GTACCTTTAACTTCCAAAAGATACATAAAAATCATGGAAGGTAATGCCAAG-3′ (SEQ ID NO: 8).

In some embodiments, the 5′ exon fragment of (E) includes a sequence having at least 80% sequence identity to 5′-TTTTTATGTATOTTTTGCGT-3′ (SEQ ID NO: 5).

In some embodiments, the 5′ exon fragment of (E) includes a sequence having at least 85%, 90%, 95%, 97%, 99%, or 100% sequence identity to 5′-TTTTTATGTATOTTTTGCGT-3′ (SEQ ID NO: 5).

In some embodiments, the 3′ exon fragment of (C) includes a sequence having at least 80% sequence identity to 5′-ATGAAGTGAACACGTTATTCAGTTCAAACGGACAGACTCCTTTTGTAACA-3′ (SEQ ID NO: 11).

In some embodiments, the 3′ exon fragment of (C) includes a sequence having at least 85%, 90%, 95%, 97%, 99%, or 100% sequence identity to 5′-ATGAAGTGAACACGTTATTCAGTTCAAACGGACAGACTCCTTTTGTAACA-3′ (SEQ ID NO: 11).

In some embodiments, the 3′ exon fragment of (C) includes a sequence having at least 80% sequence identity to 5′-ATGAAGTGAACACGTTACATAAGCTTGGAATGCAGACTCCTTTTGTAACA-3′ (SEQ ID NO: 16).

In some embodiments, the 3′ exon fragment of (C) includes a sequence having at least 85%, 90%, 95%, 97%, 99%, or 100% sequence identity to 5′-ATGAAGTGAACACGTTACATAAGCTTGGAATGCAGACTCCTTTTGTAACA-3′ (SEQ ID NO: 16).

In some embodiments, the 5′ exon fragment of (E) includes a sequence having at least 80% sequence identity to 5′-TGCATTCCAAGCTTATGAGT-3′ (SEQ ID NO: 13).

In some embodiments, the 5′ exon fragment of (E) includes a sequence having at least 85%, 90%, 95%, 97%, 99%, or 100% sequence identity to 5′-TGCATTCCAAGCTTATGAGT-3′ (SEQ ID NO: 13).

### Polyribonucleotide Cargo

A polyribonucleotide cargo described herein includes any sequence including at least one polyribonucleotide. In some embodiments, the polyribonucleotide cargo of (D) includes an expression sequence, a non-coding sequence, or an expression sequence and a non-coding sequence. In some embodiments, the polyribonucleotide cargo of (D) includes an expression sequence encoding a polypeptide. In some embodiments, the polyribonucleotide cargo of (D) includes an IRES operably linked to an expression sequence encoding a polypeptide. In some embodiments, the polyribonucleotide cargo of (D) includes an expression sequence that encodes a polypeptide that has a biological effect on a subject.

A polyribonucleotide cargo may, for example, include at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the polyribonucleotides cargo includes from 1-20,000 nucleotides, 1-10,000 nucleotides, 1-5,000 nucleotides, 100-20,000 nucleotide, 100-10,000 nucleotides, 100-5,000 nucleotides, 500-20,000 nucleotides, 500-10,000 nucleotides, 500-5,000 nucleotides, 1,000-20,000 nucleotides, 1,000-10,000 nucleotides, or 1,000-5,000 nucleotides.

In embodiments, the polyribonucleotide cargo includes one or multiple expression (or coding) sequences, wherein each expression (or coding) sequence encodes a polypeptide. In embodiments, the polyribonucleotide cargo includes one or multiple noncoding sequences. In embodiments, the polyribonucleotide cargo consists entirely of non-coding sequence(s). In embodiments, the polyribonucleotide cargo includes a combination of expression (or coding) and noncoding sequences.

In some embodiments, polyribonucleotides made as described herein are used as effectors in therapy or agriculture. For example, a circular polyribonucleotide made by the methods described herein (e.g., the cell-free methods described herein) may be administered to a subject (e.g., in a pharmaceutical, veterinary, or agricultural composition). In another example, a circular polyribonucleotide made by the methods described herein (e.g., the cell-free methods described herein) may be delivered to a cell. In some embodiments, the polyribonucleotide includes any feature, or any combination of features as disclosed in International Patent Publication No. WO2019/118919, which is hereby incorporated by reference in its entirety.

### Polypeptide Expression Sequences

In some embodiments, the polyribonucleotide described herein (e.g., the polyribonucleotide cargo of the circular polyribonucleotide) includes one or more expression (or coding) sequences, wherein each expression sequence encodes a polypeptide. In some embodiments, the circular polyribonucleotide includes two, three, four, five, six, seven, eight, nine, ten or more expression (or coding) sequences.

Each encoded polypeptide may be linear or branched. In various embodiments, the polypeptide has a length from about 5 to about 40,000 amino acids, about 15 to about 35,000 amino acids, about 20 to about 30,000 amino acids, about 25 to about 25,000 amino acids, about 50 to about 20,000 amino acids, about 100 to about 15,000 amino acids, about 200 to about 10,000 amino acids, about 500 to about 5,000 amino acids, about 1,000 to about 2,500 amino acids, or any range therebetween. In some embodiments, the polypeptide has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, or less may be useful.

Polypeptides included herein may include naturally occurring polypeptides or non-naturally occurring polypeptides. In some embodiments, the polypeptide is or includes a functional fragment or variant of a reference polypeptide (e.g., an enzymatically active fragment or variant of an enzyme). For example, the polypeptide may be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide. In some instances, the polypeptide may have at least 50% (e.g., at least 50%, 60%, 70%, 80%, 90%, 95%, 97%, 99%, or greater) identity to a protein of interest.

Some examples of a polypeptide include, but are not limited to, a fluorescent tag or marker, an antigen, a therapeutic polypeptide, or a polypeptide for agricultural applications.

A therapeutic polypeptide may be a hormone, a neurotransmitter, a growth factor, an enzyme (e.g., oxidoreductase, metabolic enzyme, mitochondrial enzyme, oxygenase, dehydrogenase, ATP-independent enzyme, lysosomal enzyme, desaturase), a cytokine, an antigen binding polypeptide (e.g., antigen binding antibody or antibody-like fragments, such as single chain antibodies, nanobodies or other Ig heavy chain or light chain containing polypeptides), an Fc fusion protein, an anticoagulant, a blood factor, a bone morphogenetic protein, an interferon, an interleukin, and a thrombolytic.

A polypeptide for agricultural applications may be a bacteriocin, a lysin, an antimicrobial polypeptide, an antifungal polypeptide, a nodule C-rich peptide, a bacteriocyte regulatory peptide, a peptide toxin, a pesticidal polypeptide (e.g., insecticidal polypeptide or nematocidal polypeptide), an antigen binding polypeptide (e.g., antigen binding antibody or antibody-like fragments, such as single chain antibodies, nanobodies or other Ig heavy chain or light chain containing polypeptides), an enzyme (e.g., nuclease, amylase, cellulase, peptidase, lipase, chitinase), a peptide pheromone, and a transcription factor.

In some cases, the circular polyribonucleotide expresses a non-human protein.

In some embodiments, the circular polyribonucleotide expresses an antibody, e.g., an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can include more than one expression (or coding) sequence, each of which expresses a portion of an antibody, and the sum of which can constitute the antibody. In some cases, the circular polyribonucleotide includes one expression sequence coding for the heavy chain of an antibody, and another expression sequence coding for the light chain of the antibody. In some cases, when the circular polyribonucleotide is expressed in a cell or a cell-free environment, the light chain and heavy chain can be subject to appropriate modification, folding, or other post-translation modification to form a functional antibody.

In embodiments, polypeptides include multiple polypeptides, e.g., multiple copies of one polypeptide sequence, or multiple different polypeptide sequences. In embodiments, multiple polypeptides are connected by linker amino acids or spacer amino acids.

In embodiments, the polynucleotide cargo includes a sequence encoding a signal peptide. Many signal peptide sequences have been described, for example, the Tat (Twin-arginine translocation) signal sequence is typically an N-terminal peptide sequence containing a consensus SRR×FLK “twin-arginine” motif, which serves to translocate a folded protein containing such a Tat signal peptide across a lipid bilayer. See also, e.g., the Signal Peptide Database publicly available at www[dot]signalpeptide[dot]de. Signal peptides are also useful for directing a protein to specific organelles; see, e.g., the experimentally determined and computationally predicted signal peptides disclosed in the Spdb signal peptide database, publicly available at proline[dot]bic[dot]nus[dot]edu[dot]sg/spdb.

In embodiments, the polynucleotide cargo includes sequence encoding a cell-penetrating peptide (CPP). Hundreds of CPP sequences have been described; see, e.g., the database of cell-penetrating peptides, CPPsite, publicly available at crdd[dot]osdd[dot]net/raghava/cppsite/. An example of a commonly used CPP sequence is a poly-arginine sequence, e.g., octoarginine or nonoarginine, which can be fused to the C-terminus of the CGI peptide.

In embodiments, the polynucleotide cargo includes sequence encoding a self-assembling peptide; see, e.g., Miki et al. (2021) Nature Communications, 21:3412, DOI: 10.1038/s41467-021-23794-6.

In some embodiments, the expression (or coding) sequence includes a poly-A sequence (e.g., at the 3′ end of an expression sequence). In some embodiments, the length of a poly-A sequence is greater than 10 nucleotides in length. In one embodiment, the poly-A sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A sequence is designed according to the descriptions of the poly-A sequence in [0202]-[0204] of International Patent Publication No. WO2019/118919A1, which is incorporated herein by reference in its entirety. In some embodiments, the expression sequence lacks a poly-A sequence (e.g., at the 3′ end of an expression sequence).

In some embodiments, a circular polyribonucleotide includes a polyA, lacks a polyA, or has a modified polyA to modulate one or more characteristics of the circular polyribonucleotide. In some embodiments, the circular polyribonucleotide lacking a polyA or having modified polyA improves one or more functional characteristics, e.g., immunogenicity (e.g., the level of one or more marker of an immune or inflammatory response), half-life, and/or expression efficiency.

### Therapeutic Polypeptides

In some embodiments, the circular polyribonucleotide described herein (e.g., the polyribonucleotide cargo of the circular polyribonucleotide) includes at least one expression sequence encoding a therapeutic polypeptide. A therapeutic polypeptide is a polypeptide that when administered to or expressed in a subject provides some therapeutic benefit. Administration to a subject or expression in a subject of a therapeutic polypeptide may be used to treat or prevent a disease, disorder, or condition or a symptom thereof. In some embodiments, the circular polyribonucleotide encodes two, three, four, five, six, seven, eight, nine, ten or more therapeutic polypeptides.

In some embodiments, the circular polyribonucleotide includes an expression sequence encoding a therapeutic protein. The protein may treat the disease in the subject in need thereof. In some embodiments, the therapeutic protein can compensate for a mutated, under-expressed, or absent protein in the subject in need thereof. In some embodiments, the therapeutic protein can target, interact with, or bind to a cell, tissue, or virus in the subject in need thereof.

A therapeutic polypeptide can be a polypeptide that can be secreted from a cell, or localized to the cytoplasm, nucleus, or membrane compartment of a cell.

A therapeutic polypeptide may be a hormone, a neurotransmitter, a growth factor, an enzyme (e.g., oxidoreductase, metabolic enzyme, mitochondrial enzyme, oxygenase, dehydrogenase, ATP-independent enzyme, lysosomal enzyme, desaturase), a cytokine, a transcription factor, an antigen binding polypeptide (e.g., antigen binding antibody or antibody-like fragments, such as single chain antibodies, nanobodies or other Ig heavy chain or light chain containing polypeptides), an Fc fusion protein, an anticoagulant, a blood factor, a bone morphogenetic protein, an interferon, an interleukin, a thrombolytic, an antigen (e.g., a tumor, viral, or bacterial antigen), a nuclease (e.g., an endonuclease such as a Cas protein, e.g., Cas9), a membrane protein (e.g., a chimeric antigen receptor (CAR), a T cell receptor (TCR), a TCR complex protein, a transmembrane receptor, a G-protein-coupled receptor (GPCR), a receptor tyrosine kinase (RTK), an antigen receptor, an ion channel, or a membrane transporter), a secreted protein, a gene editing protein (e.g., a CRISPR-Cas, TALEN, or zinc finger), or a gene writing protein (see, e.g., International Patent Publication No. WO2020/047124, incorporated in its entirety herein by reference).

In some embodiments, the therapeutic polypeptide is an antibody, e.g., a full-length antibody, an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can include more than one expression sequence, each of which expresses a portion of an antibody, and the sum of which can constitute the antibody. In some cases, the circular polyribonucleotide includes one expression sequence coding for the heavy chain of an antibody, and another expression sequence coding for the light chain of the antibody. When the circular polyribonucleotide is expressed in a cell, the light chain and heavy chain can be subject to appropriate modification, folding, or other post-translation modification to form a functional antibody.

In some embodiments, circular polyribonucleotides made as described herein are used as effectors in therapy or agriculture. For example, a circular polyribonucleotide made by the methods described herein (e.g., the cell-free methods described herein) may be administered to a subject (e.g., in a pharmaceutical, veterinary, or agricultural composition). In embodiments, the subject is a vertebrate animal (e.g., mammal, bird, fish, reptile, or amphibian). In embodiments, the subject is a human. In embodiments, the method subject is a non-human mammal. In embodiments, the subject is a non-human mammal such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusc. In embodiments, the subject is an invertebrate agricultural pest or an invertebrate that is parasitic on an invertebrate or vertebrate host. In embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses.

### Secreted Polypeptide Effectors

In some embodiments, the circular polyribonucleotide described herein (e.g., the polyribonucleotide cargo of the circular polyribonucleotide) includes at least one coding sequence encoding a secreted polypeptide effector. Exemplary secreted polypeptide effectors or proteins that may be expressed include, e.g., cytokines and cytokine receptors, polypeptide hormones and receptors, growth factors, clotting factors, therapeutic replacement enzymes and therapeutic non-enzymatic effectors, regeneration, repair, and fibrosis factors, transformation factors, and proteins that stimulate cellular regeneration, non-limiting examples of which are described herein, e.g., in the tables below.

### Cytokines and Cytokine Receptors

In some embodiments, an effector described herein comprises a cytokine of Table 1, or a functional variant or fragment thereof, e.g., a protein having at least 80%, 85%, 90%, 95%, 967%, 98%, 99% identity to a protein sequence disclosed in Table 1 by reference to its UniProt ID. In some embodiments, the functional variant binds to the corresponding cytokine receptor with a Kd of no more than 10%, 20%, 30%, 40%, or 50% higher or lower than the Kd of the corresponding wild-type cytokine for the same receptor under the same conditions. In some embodiments, the effector comprises a fusion protein comprising a first region (e.g., a cytokine polypeptide of Table 1 or a functional variant or fragment thereof) and a second, heterologous region. In some embodiments, the first region is a first cytokine polypeptide of Table 1. In some embodiments, the second region is a second cytokine polypeptide of Table 1, wherein the first and second cytokine polypeptides form a cytokine heterodimer with each other in a wild-type cell. In some embodiments, the polypeptide of Table 1 or functional variant thereof comprises a signal sequence, e.g., a signal sequence that is endogenous to the effector, or a heterologous signal sequence.

In some embodiments, an effector described herein comprises an antibody or fragment thereof that binds a cytokine of Table 1. In some embodiments, the antibody molecule comprises a signal sequence.

TABLE 1
Exemplary cytokines and cytokine receptors

| Cytokine | Cytokine receptor(s) | Entrez Gene ID¹ | UniProt ID² |
|---|---|---|---|
| IL-1α, IL-1β, or a | IL-1 type 1 receptor, | 3552, 3553 | P01583, P01584 |
| heterodimer thereof | IL-1 type 2 receptor | | |
| IL-1Ra | IL-1 type 1 receptor, | 3454, 3455 | P17181, P48551 |
| | IL-1 type 2 receptor | | |
| IL-2 | IL-2R | 3558 | P60568 |
| IL-3 | IL-3 receptor α + β c (CD131) | 3562 | P08700 |
| IL-4 | IL-4R type I, IL-4R type II | 3565 | P05112 |
| IL-5 | IL-5R | 3567 | P05113 |
| IL-6 | IL-6R (sIL-6R) gp130 | 3569 | P05231 |
| IL-7 | IL-7R and sIL-7R | 3574 | P13232 |
| IL-8 | CXCR1 and CXCR2 | 3576 | P10145 |
| IL-9 | IL-9R | 3578 | P15248 |
| IL-10 | IL-10R1/IL-10R2 complex | 3586 | P22301 |
| IL-11 | IL-11Rα 1 gp130 | 3589 | P20809 |
| IL-12 (e.g., p35, p40, | IL-12Rβ1 and IL-12Rβ2 | 3593, 3592 | P29459, P29460 |
| or a heterodimer | | | |
| thereof) | | | |
| IL-13 | IL-13R1α1 and IL-13R1α2 | 3596 | P35225 |
| IL-14 | IL-14R | 30685 | P40222 |
| IL-15 | IL-15R | 3600 | P40933 |
| IL-16 | CD4 | 3603 | Q14005 |
| IL-17A | IL-17RA | 3605 | Q16552 |
| IL-17B | IL-17RB | 27190 | Q9UHF5 |
| IL-17C | IL-17RA to IL-17RE | 27189 | Q9P0M4 |
| IL-17D | SEF | 53342 | Q8TAD2 |
| IL-17F | IL-17RA, IL-17RC | 112744 | Q96PD4 |
| IL-18 | IL-18 receptor | 3606 | Q14116 |
| IL-19 | IL-20R1/IL-20R2 | 29949 | Q9UHD0 |
| IL-20 | L-20R1/IL-20R2 and | 50604 | Q9NYY1 |
| | IL-22R1/IL-20R2 | | |
| IL-21 | IL-21R | 59067 | Q9HBE4 |
| IL-22 | IL-22R | 50616 | Q9GZX6 |
| IL-23 (e.g., p19, p40, | IL-23R | 51561 | Q9NPF7 |
| or a heterodimer | | | |
| thereof) | | | |
| IL-24 | IL-20R1/IL-20R2 and | 11009 | Q13007 |
| | IL-22R1/IL-20R2 | | |
| IL-25 | IL-17RA and IL-17RB | 64806 | Q9H293 |
| IL-26 | IL-10R2 chain and | 55801 | Q9NPH9 |
| | IL-20R1 chain | | |
| IL-27 (e.g., p28, EBI3, | WSX-1 and gp130 | 246778 | Q8NEV9 |
| or a heterodimer | | | |
| thereof) | | | |
| IL-28A, IL-28B, | IL-28R1/IL-10R2 | 282617, 282618 | Q8IZI9, Q8IU54 |
| and IL29 | | | |
| IL-30 | IL6R/gp130 | 246778 | Q8NEV9 |
| IL-31 | IL-31RA/OSMRβ | 386653 | Q6EBC2 |
| IL-32 | | 9235 | P24001 |
| IL-33 | ST2 | 90865 | O95760 |
| IL-34 | Colony-stimulating | 146433 | Q6ZMJ4 |
| | factor 1 receptor | | |
| IL-35 (e.g., p35, EBI3, | IL-12Rβ2/gp130; | 10148 | Q14213 |
| or a heterodimer | IL-12Rβ2/IL-12Rβ2; | | |
| thereof) | gp130/gp130 | | |
| IL-36 | IL-36Ra | 27179 | Q9UHA7 |
| IL-37 | IL-18Rα and IL-18BP | 27178 | Q9NZH6 |
| IL-38 | IL-1R1, IL-36R | 84639 | Q8WWZ1 |
| IFN-α | IFNAR | 3454 | P17181 |
| IFN-β | IFNAR | 3454 | P17181 |
| IFN-γ | IFNGR1/IFNGR2 | 3459 | P15260 |
| TGF-β | TβR-I and TβR-II | 7046, 7048 | P36897, P37173 |
| TNF-α | TNFR1, TNFR2 | 7132, 7133 | P19438, P20333 |

¹Sequence available on the NCBI database on the world wide web internet site “ncbi.nlm.nih.gov/gene”; Maglott D, et al. Gene: a gene-centered information resource at NCBI. Nucleic Acids Res. 2014. pii: gku1055.
²Sequence available on the Uniprot database on the world wide web internet site “uniprot.org/uniprot/”; UniProt: the universal protein knowledgebase in 2021. Nucleic Acids Res. 49: D1 (2021).

### Polypeptide Hormones and Receptors

In some embodiments, an effector described herein comprises a hormone of Table 2, or a functional variant thereof, e.g., a protein having at least 80%, 85%, 90%, 95%, 967%, 98%, 99% identity to a protein sequence disclosed in Table 2 by reference to its UniProt ID. In some embodiments, the functional variant binds to the corresponding receptor with a Kd of no more than 10%, 20%, 30%, 40%, or 50% higher than the Kd of the corresponding wild-type hormone for the same receptor under the same conditions. In some embodiments, the polypeptide of Table 2 or functional variant thereof comprises a signal sequence, e.g., a signal sequence that is endogenous to the effector, or a heterologous signal sequence.

In some embodiments, an effector described herein comprises an antibody molecule (e.g., an scFv) that binds a hormone of Table 2. In some embodiments, an effector described herein comprises an antibody molecule (e.g., an scFv) that binds a hormone receptor of Table 2. In some embodiments, the antibody molecule comprises a signal sequence.

TABLE 2
Exemplary polypeptide hormones and receptors

| | | Entrez | UniProt |
|---|---|---|---|
| Hormone | Receptor | Gene ID¹ | ID² |
| Natriuretic Peptide, e.g., | NPRA, NPRB, NPRC | 4878 | P01160 |
| Atrial Natriuretic | | | |
| Peptide (ANP) | | | |
| Brain Natriuretic | NPRA, NPRB | 4879 | P16860 |
| Peptide (BNP) | | | |
| C-type natriuretic | NPRB | 4880 | P23582 |
| peptide (CNP) | | | |
| Growth hormone (GH) | GHR | 2690 | P10912 |
| Prolactin (PRL) | PRLR | 5617 | P01236 |
| Thyroid-stimulating | TSH receptor | 7253 | P16473 |
| hormone (TSH) | | | |
| Adrenocorticotropic | ACTH receptor | 5443 | P01189 |
| hormone (ACTH) | | | |
| Follicle-stimulating | FSHR | 2492 | P23945 |
| hormone (FSH) | | | |
| Luteinizing | LHR | 3973 | P22888 |
| hormone (LH) | | | |
| Antidiuretic | Vasopressin receptors, | 554 | P30518 |
| hormone (ADH) | e.g., V2; AVPR1A; | | |
| | AVPR1B; AVPR3; AVPR2 | | |
| Oxytocin | OXTR | 5020 | P01178 |
| Calcitonin | Calcitonin receptor (CT) | 796 | P01258 |
| Parathyroid hormone (PTH) | PTH1R and PTH2R | 5741 | P01270 |
| Insulin | Insulin receptor (IR) | 3630 | P01308 |
| Glucagon | Glucagon receptor | 2641 | P01275 |
| GIP | GIPR | 2695 | P09681 |
| Fibroblast growth | FGFR4 | 9965 | O95750 |
| factor 19 (FGF19) | | | |
| Fibroblast growth | FGFR1c, 2c, 3c | 26291 | Q9NSA1 |
| factor 21 (FGF21) | | | |
| Fibroblast growth | FGFR1, 2, 4 | 8074 | Q9GZV9 |
| factor 23 (FGF23) | | | |
| Melanocyte- | MC1R, MC4R, MC5R | | |
| stimulating | | | |
| hormone (alpha- MSH) | | | |
| Melanocyte- | MC4R | | |
| stimulating | | | |
| hormone (beta- MSH) | | | |
| Melanocyte- | MC1R, MC3R, MC4R, | | |
| stimulating | MC5R | | |
| hormone (gamma- MSH) | | | |
| Proopiomelanocortin POMC | MC1R, MC3R, MC4R, | 5443 | P01189 |
| (alpha- beta-, gamma-, | MC5R | | |
| MSH precursor) | | | |
| Glycoprotein hormones | | 1081 | P01215 |
| alpha chain (CGA) | | | |
| Follicle-stimulating | FSHR | 2488 | P01225 |
| hormone beta (FSHB) | | | |
| Leptin | LEPR | 3952 | P41159 |
| Ghrelin | GHSR | 51738 | Q9UBU3 |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot/”; UniProt: the universal protein knowledgebase in 2021. Nucleic Acids Res. 49: D1 (2021).

### Growth Factors

In some embodiments, an effector described herein comprises a growth factor of Table 3, or a functional variant thereof, e.g., a protein having at least 80%, 85%, 90%, 95%, 967%, 98%, 99% identity to a protein sequence disclosed in Table 3 by reference to its UniProt ID. In some embodiments, the functional variant binds to the corresponding receptor with a Kd of no more than 10%, 20%, 30%, 40%, or 50% higher than the Kd of the corresponding wild-type growth factor for the same receptor under the same conditions. In some embodiments, the polypeptide of Table 3 or functional variant thereof comprises a signal sequence, e.g., a signal sequence that is endogenous to the effector, or a heterologous signal sequence.

In some embodiments, an effector described herein comprises an antibody or fragment thereof that binds a growth factor of Table 3. In some embodiments, an effector described herein comprises an antibody molecule (e.g., an scFv) that binds a growth factor receptor of Table 3. In some embodiments, the antibody molecule comprises a signal sequence.

TABLE 3
Exemplary growth factors

| | | Entrez | UniProt |
|---|---|---|---|
| Growth Factor | | Gene ID¹ | ID² |
| PDGF family | | | |
| PDGF (e.g., PDGF-1, | PDGF receptor, | 5156 | P16234 |
| PDGF-2, or a | e.g., PDGFRα, | | |
| heterodimer thereof) | PDGFRβ | | |
| CSF-1 | CSF1R | 1435 | P09603 |
| SCF | CD117 | 3815 | P10721 |
| VEGF family | | | |
| VEGF (e.g., isoforms | VEGFR-1, | 2321 | P17948 |
| VEGF 121, VEGF 165, | VEGFR-2 | | |
| VEGF 189, and VEGF | | | |
| 206) | | | |
| VEGF-B | VEGFR-1 | 2321 | P17949 |
| VEGF-C | VEGFR-2 and | 2324 | P35916 |
| | VEGFR -3 | | |
| PIGF | VEGFR-1 | 5281 | Q07326 |
| EGF family | | | |
| EGF | EGFR | 1950 | P01133 |
| TGF-α | EGFR | 7039 | P01135 |
| amphiregulin | EGFR | 374 | P15514 |
| HB-EGF | EGFR | 1839 | Q99075 |
| betacellulin | EGFR, ErbB-4 | 685 | P35070 |
| epiregulin | EGFR, ErbB-4 | 2069 | O14944 |
| Heregulin | EGFR, ErbB-4 | 3084 | Q02297 |
| FGF family | | | |
| FGF-1, FGF-2, FGF-3, | FGFR1, FGFR2, | 2246, 2247, 2248, | P05230, P09038, |
| FGF-4, FGF-5, FGF-6, | FGFR3, and | 2249, 2250, 2251, | P11487, P08620, |
| FGF-7, FGF-8, FGF-9 | FGFR4 | 2252, 2253, 2254 | P12034, P10767, |
| | | | P21781, P55075, |
| | | | P31371 |
| Insulin family | | | |
| Insulin | IR | 3630 | P01308 |
| IGF-I | IGF-I receptor, | 3479 | P05019 |
| | IGF-II receptor | | |
| IGF-II | IGF-II receptor | 3481 | P01344 |
| HGF family | | | |
| HGF | MET receptor | 3082 | P14210 |
| MSP | RON | 4485 | P26927 |
| Neurotrophin family | | | |
| NGF | LNGFR, trkA | 4803 | P01138 |
| BDNF | trkB | 627 | P23560 |
| NT-3 | trkA, trkB, trkC | 4908 | P20783 |
| NT-4 | trkA, trkB | 4909 | P34130 |
| NT-5 | trkA, trkB | 4909 | P34130 |
| Angiopoietin family | | | |
| ANGPT1 | HPK-6/TEK | 284 | Q15389 |
| ANGPT2 | HPK-6/TEK | 285 | O15123 |
| ANGPT3 | HPK-6/TEK | 9068 | O95841 |
| ANGPT4 | HPK-6/TEK | 51378 | Q9Y264 |
| ANGPTL2 | LILRB2 & | 23452 | Q9UKU9 |
| | integrin α5β1 | | |
| ANGPTL3 | LPL | 27329 | Q9Y5C1 |
| ANGPTL4 | | 51129 | Q9BY76 |
| ANGPTL8 | PirB | 55908 | Q6UXH0 |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot”; UniProt: the universal protein knowledgebase in 2021. Nucleic Acids Res. 49: D1 (2021).

### Clotting Factors

In some embodiments, an effector described herein comprises a polypeptide of Table 4, or a functional variant thereof, e.g., a protein having at least 80%, 85%, 90%, 95%, 967%, 98%, 99% identity to a protein sequence disclosed in Table 4 by reference to its UniProt ID. In some embodiments, the functional variant catalyzes the same reaction as the corresponding wild-type protein, e.g., at a rate no less than 10%, 20%, 30%, 40%, or 50% lower or higher than the wild-type protein. In some embodiments, the polypeptide of Table 4 or functional variant thereof comprises a signal sequence, e.g., a signal sequence that is endogenous to the effector, or a heterologous signal sequence.

TABLE 4
Clotting-associated factors

| | | Entrez | UniProt |
|---|---|---|---|
| Effector | Indication | Gene ID¹ | ID² |
| Factor I | Afibrinogenomia | 2243, | P02671, |
| (fibrinogen) | | 2266, 2244 | P02679, P02675 |
| Factor II | Factor II Deficiency | 2147 | P00734 |
| Factor IX | Hemophilia B | 2158 | P00740 |
| Factor V | Owren's disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing | 2162, 2165 | P00488, P05160 |
| | factor deficiency | | |
| vWF | von Willebrand | 7450 | P04275 |
| | disease | | |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot/”; UniProt: the universal protein knowledgebase in 2021. Nucleic Acids Res. 49: D1 (2021).

### Therapeutic Replacement Enzymes

In some embodiments, an effector described herein comprises an enzyme of Table 5, or a functional variant thereof, e.g., a protein having at least 80%, 85%, 90%, 95%, 967%, 98%, 99% identity to a protein sequence disclosed in Table 5 by reference to its UniProt ID. In some embodiments, the functional variant catalyzes the same reaction as the corresponding wild-type protein, e.g., at a rate no less or no more than 10%, 20%, 30%, 40%, or 50% lower than the wild-type protein.

TABLE 5
Exemplary enzymatic effectors for enzyme deficiency

| | | Entrez | UniProt |
|---|---|---|---|
| Effector | Deficiency | Gene ID¹ | ID² |
| 3-methylcrotonyl-CoA | 3-methylcrotonyl-CoA | 56922, 64087 | Q96RQ3, |
| carboxylase | carboxylase deficiency | | Q9HCC0 |
| Acetyl-CoA- | Mucopolysaccharidosis MPS III | 138050 | Q68CP4 |
| glucosaminide N- | (Sanfilippo's syndrome) Type III-C | | |
| acetyltransferase | | | |
| ADAMTS13 | Thrombotic Thrombocytopenia | 11093 | Q76LX8 |
| | Purpura | | |
| adenine | Adenine phosphoribosyltransferase | 353 | P07741 |
| phosphoribosyltransferase | deficiency | | |
| Adenosine deaminase | Adenosine deaminase deficiency | 100 | P00813 |
| ADP-ribose protein | Glutamyl ribose-5-phosphate | 26119, 54936 | Q5SW96, |
| hydrolase | storage disease | | Q9NX46 |
| alpha glucosidase | Glycogen storage disease type 2 | 2548 | P10253 |
| | (Pompe's disease) | | |
| Arginase | Familial hyperarginemia | 383, 384 | P05089, |
| | | | P78540 |
| Arylsulfatase A | Metachromatic leukodystrophy | 410 | P15289 |
| Cathepsin K | Pycnodysostosis | 1513 | P43235 |
| Ceramidase | Farber's disease | 125981, | Q8TDN7, |
| | (lipogranulomatosis) | 340485, 55331 | Q5QJU3, |
| | | | Q9NUN7 |
| Cystathionine B synthase | Homocystinuria | 875 | P35520 |
| Dolichol-P-mannose | Congenital disorders of N- | 8813, 54344 | O60762, |
| synthase | glycosylation CDG le | | Q9P2X0 |
| Dolicho-P- | Congenital disorders of N- | 84920 | Q5BKT4 |
| Glc:Man9GlcNAc2-PP- | glycosylation CDG lc | | |
| dolichol | | | |
| glucosyltransferase | | | |
| Dolicho-P- | Congenital disorders of N- | 10195 | Q92685 |
| Man:Man5GlcNAc2-PP- | glycosylation CDG ld | | |
| dolichol | | | |
| mannosyltransferase | | | |
| Dolichyl-P-glucose:Glc-1- | Congenital disorders of N- | 79053 | Q9BVK2 |
| Man-9-GlcNAc-2-PP- | glycosylation CDG lh | | |
| dolichyl-α-3- | | | |
| glucosyltransferase | | | |
| Dolichyl-P-mannose:Man- | Congenital disorders of N- | 79087 | Q9BV10 |
| 7-GlcNAc-2-PP- | glycosylation CDG lg | | |
| dolichyl-α-6- | | | |
| mannosyltransferase | | | |
| Factor II | Factor II Deficiency | 2147 | P00734 |
| Factor IX | Hemophilia B | 2158 | P00740 |
| Factor V | Owren's disease | 2153 | P12259 |
| Factor VIII | Hemophilia A | 2157 | P00451 |
| Factor X | Stuart-Prower Factor Deficiency | 2159 | P00742 |
| Factor XI | Hemophilia C | 2160 | P03951 |
| Factor XIII | Fibrin Stabilizing | 2162, 2165 | P00488, |
| | factor deficiency | | P05160 |
| Galactosamine-6- | Mucopolysaccharidosis MPS IV | 2588 | P34059 |
| sulfate sulfatase | (Morquio's syndrome) Type IV-A | | |
| Galactosylceramide β- | Krabbe's disease | 2581 | P54803 |
| galactosidase | | | |
| Ganglioside β- | GM1 gangliosidosis, generalized | 2720 | P16278 |
| galactosidase | | | |
| Ganglioside β- | GM2 gangliosidosis | 2720 | P16278 |
| galactosidase | | | |
| Ganglioside β- | Sphingolipidosis Type I | 2720 | P16278 |
| galactosidase | | | |
| Ganglioside β- | Sphingolipidosis Type II | 2720 | P16278 |
| galactosidase | (juvenile type) | | |
| Ganglioside β- | Sphingolipidosis Type III | 2720 | P16278 |
| galactosidase | (adult type) | | |
| Glucosidase I | Congenital disorders of N- | 2548 | P10253 |
| | glycosylation CDG llb | | |
| Glucosylceramide β- | Gaucher's disease | 2629 | P04062 |
| glucosidase | | | |
| Heparan-S-sulfate | Mucopolysaccharidosis MPS III | 6448 | P51688 |
| sulfamidase | (Sanfilippo's syndrome) | | |
| | Type III-A | | |
| homogentisate oxidase | Alkaptonuria | 3081 | Q93099 |
| Hyaluronidase | Mucopolysaccharidosis MPS IX | 3373, 8692, | Q12794, |
| | (hyaluronidase deficiency) | 8372, 23553 | Q12891, |
| | | | O43820, |
| | | | Q2M3T9 |
| Iduronate sulfate | Mucopolysaccharidosis MPS II | 3423 | P22304 |
| sulfatase | (Hunter's syndrome) | | |
| Lecithin-cholesterol | Complete LCAT deficiency, Fish-eye | 3931 | 606967 |
| acyltransferase (LCAT) | disease, atherosclerosis, | | |
| | hypercholesterolemia | | |
| Lysine oxidase | Glutaric acidemia type I | 4015 | P28300 |
| Lysosomal acid lipase | Cholesteryl ester storage disease | 3988 | P38571 |
| | (CESD) | | |
| Lysosomal acid lipase | Lysosomal acid lipase deficiency | 3988 | P38571 |
| lysosomal acid lipase | Wolman's disease | 3988 | P38571 |
| Lysosomal pepstatin- | Ceroid lipofuscinosis Late infantile | 1200 | O14773 |
| insensitive peptidase | form (CLN2, Jansky-Bielschowsky | | |
| | disease) | | |
| Mannose (Man) | Congenital disorders of N- | 4351 | P34949 |
| phosphate (P) isomerase | glycosylation CDG lb | | |
| Mannosyl-α-1,6- | Congenital disorders of N- | 4247 | Q10469 |
| glycoprotein-β-1,2-N- | glycosylation CDG lla | | |
| acetylglucosminyltransferase | | | |
| Metalloproteinase-2 | Winchester syndrome | 4313 | P08253 |
| methylmalonyl-CoA | Methylmalonic acidemia (vitamin | 4594 | P22033 |
| mutase | b12 non-responsive) | | |
| N-Acetyl | Mucopolysaccharidosis MPS VI | 411 | P15848 |
| galactosamine α-4-sulfate | (Maroteaux-Lamy syndrome) | | |
| sulfatase (arylsulfatase B) | | | |
| N-acetyl-D- | Mucopolysaccharidosis MPS III | 4669 | P54802 |
| glucosaminidase | (Sanfilippo's syndrome) | | |
| | Type III-B | | |
| N-Acetyl- | Schindler's disease Type I | 4668 | P17050 |
| galactosaminidase | (infantile severe form) | | |
| N-Acetyl- | Schindler's disease Type II | 4668 | P17050 |
| galactosaminidase | (Kanzaki disease, adult-onset form) | | |
| N-Acetyl- | Schindler's disease Type III | 4668 | P17050 |
| galactosaminidase | (intermediate form) | | |
| N-acetyl-glucosaminine- | Mucopolysaccharidosis MPS III | 2799 | P15586 |
| 6-sulfate sulfatase | (Sanfilippo's syndrome) | | |
| | Type III-D | | |
| N-acetylglucosaminyl-1- | Mucolipidosis ML III (pseudo- | 79158 | Q3T906 |
| phosphotransferase | Hurler's polydystrophy) | | |
| N-Acetylglucosaminyl-1- | Mucolipidosis ML II | 79158 | Q3T906 |
| phosphotransferase | (I-cell disease) | | |
| catalytic subunit | | | |
| N-acetylglucosaminyl-1- | Mucolipidosis ML III (pseudo- | 84572 | Q9UJJ9 |
| phosphotransferase, | Hurler's polydystrophy) | | |
| substrate-recognition | Type III-C | | |
| subunit | | | |
| N- | Aspartylglucosaminuria | 175 | P20933 |
| Aspartylglucosaminidase | | | |
| Neuraminidase 1 | Sialidosis | 4758 | Q99519 |
| (sialidase) | | | |
| Palmitoyl-protein | Ceroid lipofuscinosis Adult form | 5538 | P50897 |
| thioesterase-1 | (CLN4, Kufs' disease) | | |
| Palmitoyl-protein | Ceroid lipofuscinosis Infantile form | 5538 | P50897 |
| thioesterase-1 | (CLN1, Santavuori-Haltia disease) | | |
| Phenylalanine | Phenylketonuria | 5053 | P00439 |
| hydroxylase | | | |
| Phosphomannomutase-2 | Congenital disorders of N- | 5373 | O15305 |
| | glycosylation CDG la (solely | | |
| | neurologic and neurologic- | | |
| | multivisceral forms) | | |
| Porphobilinogen | Acute Intermittent Porphyria | 3145 | P08397 |
| deaminase | | | |
| Purine nucleoside | Purine nucleoside phosphorylase | 4860 | P00491 |
| phosphorylase | deficiency | | |
| pyrimidine 5′ nucleotidase | Hemolytic anemia and/or pyrimidine | 51251 | Q9H0P0 |
| | 5′ nucleotidase deficiency | | |
| Sphingomyelinase | Niemann-Pick disease type A | 6609 | P17405 |
| Sphingomyelinase | Niemann-Pick disease type B | 6609 | P17405 |
| Sterol 27-hydroxylase | Cerebrotendinous xanthomatosis | 1593 | Q02318 |
| | (cholestanol lipidosis) | | |
| Thymidine phosphorylase | Mitochondrial neurogastrointestinal | 1890 | P19971 |
| | encephalomyopathy (MNGIE) | | |
| Trihexosylceramide α- | Fabry's disease | 2717 | P06280 |
| galactosidase | | | |
| tyrosinase, e.g., OCA1 | albinism, e.g., ocular albinism | 7299 | P14679 |
| UDP-GlcNAc:dolichyl-P | Congenital disorders of N- | 1798 | Q9H3H5 |
| NAcGlc | glycosylation CDG lj | | |
| phosphotransferase | | | |
| UDP-N- | Sialuria French type | 10020 | Q9Y223 |
| acetylglucosamine-2- | | | |
| epimerase/N- | | | |
| acetylmannosamine | | | |
| kinase, sialin | | | |
| Uricase | Lesch-Nyhan syndrome, gout | 391051 | No protein |
| uridine diphosphate | Crigler-Najjar syndrome | 54658 | P22309 |
| glucuronyl-transferase | | | |
| (e.g., UGT1A1) | | | |
| α-1,2- | Congenital disorders of N- | 79796 | Q9H6U8 |
| Mannosyltransferase | glycosylation CDG ll (608776) | | |
| α-1,2- | Congenital disorders of N- | 79796 | Q9H6U8 |
| Mannosyltransferase | glycosylation, type I (pre-Golgi | | |
| | glycosylation defects) | | |
| α-1,3- | Congenital disorders of N- | 440138 | Q2TAA5 |
| Mannosyltransferase | glycosylation CDG li | | |
| α-D-Mannosidase | a-Mannosidosis, type I | 10195 | Q92685 |
| | (severe) or II (mild) | | |
| α-L-Fucosidase | Fucosidosis | 4123 | Q9NTJ4 |
| α-I-Iduronidase | Mucopolysaccharidosis MPS I | 2517 | P04066 |
| | H/S (Hurler-Scheie syndrome) | | |
| α-I-Iduronidase | Mucopolysaccharidosis MPS I-H | 3425 | P35475 |
| | (Hurler's syndrome) | | |
| α-I-Iduronidase | Mucopolysaccharidosis MPS I-S | 3425 | P35475 |
| | (Scheie's syndrome) | | |
| β-1,4- | Congenital disorders of N- | 3425 | P35475 |
| Galactosyltransferase | glycosylation CDG lld | | |
| β-1,4- | Congenital disorders of N- | 2683 | P15291 |
| Mannosyltransferase | glycosylation CDG lk | | |
| β-D-Mannosidase | β-Mannosidosis | 56052 | Q9BT22 |
| β-Galactosidase | Mucopolysaccharidosis MPS IV | 4126 | O00462 |
| | (Morquio's syndrome) | | |
| | Type IV-B | | |
| β-Glucuronidase | Mucopolysaccharidosis MPS | 2720 | P16278 |
| | VII (Sly's syndrome) | | |
| β-Hexosaminidase A | Tay-Sachs disease | 2990 | P08236 |
| β-Hexosaminidase B | Sandhoff's disease | 3073 | P06865 |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot/”; UniProt: the universal protein knowledgebase in 2021. Nucleic Acids Res. 49: D1 (2021).

### Other Non-Enzymatic Effectors

In some embodiments, a therapeutic polypeptide described herein comprises a polypeptide of Table 6, or a functional variant thereof, e.g., a protein having at least 80%, 85%, 90%, 95%, 967%, 98%, 99% identity to a protein sequence disclosed in Table 6 by reference to its UniProt ID.

TABLE 6
Exemplary non-enzymatic effectors and corresponding indications

| | | Entrez | UniProt |
|---|---|---|---|
| Effector | Indication | Gene ID¹ | ID² |
| Survival motor | spinal muscular atrophy | 6606 | Q16637 |
| neuron protein | | | |
| (SMN) | | | |
| Dystrophin | muscular dystrophy (e.g., Duchenne | 1756 | P11532 |
| | muscular dystrophy or Becker | | |
| | muscular dystrophy) | | |
| Complement | Complement Factor I | 3426 | P05156 |
| protein, e.g., | deficiency | | |
| Complement | | | |
| factor C1 | | | |
| Complement | Atypical hemolytic | 3075 | P08603 |
| factor H | uremic syndrome | | |
| Cystinosin | Cystinosis | 1497 | O60931 |
| (lysosomal | | | |
| cystine | | | |
| transporter) | | | |
| Epididymal | Niemann-Pick disease Type C2 | 10577 | P61916 |
| secretory protein | | | |
| 1 (HE1; NPC2 | | | |
| protein) | | | |
| GDP-fucose | Congenital disorders of N-glycosylation | 55343 | Q96A29 |
| transporter-1 | CDG llc (Rambam-Hasharon syndrome) | | |
| GM2 activator | GM2 activator protein deficiency (Tay- | 2760 | Q17900 |
| protein | Sachs disease AB variant, GM2A) | | |
| Lysosomal | Ceroid lipofuscinosis Juvenile | 1207 | Q13286 |
| transmembrane | form (CLN3, Batten disease, | | |
| CLN3 protein | Vogt-Spielmeyer disease) | | |
| Lysosomal | Ceroid lipofuscinosis Variant | 1203 | O75503 |
| transmembrane | late infantile form, | | |
| CLN5 protein | Finnish type (CLN5) | | |
| Na phosphate | Infantile sialic acid | 26503 | Q9NRA2 |
| cotransporter, | storage disorder | | |
| sialin | | | |
| Na phosphate | Sialuria Finnish type | 26503 | Q9NRA2 |
| cotransporter, | (Salla disease) | | |
| sialin | | | |
| NPC1 protein | Niemann-Pick disease Type C1/Type D | 4864 | O15118 |
| Oligomeric Golgi | Congenital disorders of N- | 91949 | P83436 |
| complex-7 | glycosylation CDG lle | | |
| Prosaposin | Prosaposin deficiency | 5660 | P07602 |
| Protective | Galactosialidosis (Goldberg's syndrome, | 5476 | P10619 |
| protein/cathepsin | combined neuraminidase and β- | | |
| A (PPCA) | galactosidase deficiency) | | |
| Protein involved | Congenital disorders of N- | 9526 | O75352 |
| in mannose-P- | glycosylation CDG lf | | |
| dolichol utilization | | | |
| Saposin B | Saposin B deficiency (sulfatide | 5660 | P07602 |
| | activator deficiency) | | |
| Saposin C | Saposin C deficiency (Gaucher's | 5660 | P07602 |
| | activator deficiency) | | |
| Sulfatase- | Mucosulfatidosis (multiple | 285362 | Q8NBK3 |
| modifying | sulfatase deficiency) | | |
| factor-1 | | | |
| Transmembrane | Ceroid lipofuscinosis Variant | 54982 | Q9NWW5 |
| CLN6 protein | late infantile form (CLN6) | | |
| Transmembrane | Ceroid lipofuscinosis Progressive | 2055 | Q9UBY8 |
| CLN8 protein | epilepsy with intellectual disability | | |
| vWF | von Willebrand disease | 7450 | P04275 |
| Factor I | Afibrinogenomia | 2243, | P02671, |
| (fibrinogen) | | 2244, 2266 | P02675, |
| | | | P02679 |
| erythropoietin | | | |
| (hEPO) | | | |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot”; UniProt: the universal protein knowledgebase in 2021. Nucleic Acids Res. 49: D1 (2021).

### Regeneration, Repair and Fibrosis Factors

Therapeutic polypeptides described herein also include growth factors, e.g., as disclosed in Table 7, or functional variants thereof, e.g., a protein having at least 80%, 85%, 90%, 95%, 967%, 98%, 99% identity to a protein sequence disclosed in Table 7 by reference to its NCBI protein accession number. Also included are antibodies or fragments thereof against such growth factors, or miRNAs that promote regeneration and repair.

TABLE 7
Exemplary Regeneration, Repair, and Fibrosis Factors

| | NCBI Gene | NCBI Protein |
|---|---|---|
| Target | accession # ¹ | accession # ² |
| VEGF-A | NG_008732 | NP_001165094 |
| NRG-1 | NG_012005 | NP_001153471 |
| FGF2 | NG_029067 | NP_001348594 |
| FGF1 | Gene ID: 2246 | NP_001341882 |
| miR199-3p | MIMAT0000232 | n/a |
| miR590-3p | MIMAT0004801 | n/a |
| miR17-92 | MI0000071 | On the world wide web internet site |
| | | “ncbi.nlm.nih.gov/pmc/ |
| | | articles/PMC2732113/figure/F1/” |
| miR222 | MI0000299 | n/a |
| miR302-367 | MIR302A And | On the world wide web internet site |
| | MIR367 | “ncbi.nlm.nih.gov/pmc/ |
| | | articles/PMC4400607/” |

¹ Sequence available on the world wide web internet site “ncbi.nlm.nih.gov/gene” (Maglott D, et al. Gene: a gene-centered information resource at NCBI. Nucleic Acids Res. 2014. Pii: gku1055.)
² Sequence available on the world wide web internet site “ncbi.nlm.nih.gov/protein/”

### Transformation Factors

Therapeutic polypeptides described herein also include transformation factors, e.g., protein factors that transform fibroblasts into differentiated cell e.g., factors disclosed in Table 8 or functional variants thereof, e.g., a protein having at least 80%, 85%, 90%, 95%, 967%, 98%, 99% identity to a protein sequence disclosed in Table 8 by reference to its UniProt ID.

TABLE 8
Polypeptides indicated for organ repair by transforming fibroblasts

| | NCBI Gene | NCBI Protein |
|---|---|---|
| Target | accession # ¹ | accession # ² |
| MESP1 | Gene ID: 55897 | EAX02066 |
| ETS2 | GeneID: 2114 | NP_005230 |
| HAND2 | GeneID: 9464 | NP_068808 |
| MYOCARDIN | GeneID: 93649 | NP_001139784 |
| ESRRA | Gene ID: 2101 | AAH92470 |
| miR1 | MI0000651 | n/a |
| miR133 | MI000450 | n/a |
| TGFb | GeneID: 7040 | NP_000651.3 |
| WNT | Gene ID: 7471 | NP_005421 |
| JAK | Gene ID: 3716 | NP_001308784 |
| NOTCH | GeneID: 4851 | XP_011517019 |

¹ Sequence available on the world wide web internet site “ncbi.nlm.nih.gov/gene” (Maglott D, et al. Gene: a gene-centered information resource at NCBI. Nucleic Acids Res. 2014. Pii: gku1055.)
² Sequence available on the world wide web internet site “ncbi.nlm.nih.gov/protein/”

Proteins that Stimulate Cellular Regeneration

Therapeutic polypeptides described herein also include proteins that stimulate cellular regeneration e.g., proteins disclosed in Table 9 or functional variants thereof, e.g., a protein having at least 80%, 85%, 90%, 95%, 967%, 98%, 99% identity to a protein sequence disclosed in Table 9 by reference to its UniProt ID.

TABLE 9
Exemplary proteins that stimulate cellular regeneration

| Target | Gene accession # ¹ | Protein accession # ² |
|---|---|---|
| MST1 | NG 016454 | NP_066278 |
| STK30 | Gene ID: 26448 | NP_036103 |
| MST2 | Gene ID: 6788 | NP_006272 |
| SAV1 | Gene ID: 60485 | NP_068590 |
| LATS1 | Gene ID: 9113 | NP_004681 |
| LATS2 | Gene ID: 26524 | NP_055387 |
| YAP1 | NG 029530 | NP_001123617 |
| CDKN2b | NG 023297 | NP_004927 |
| CDKN2a | NG 007485 | NP 478102 |

¹ Sequence available on the world wide web internet site “ncbi.nlm.nih.gov/gene” (Maglott D, et al. Gene: a gene-centered information resource at NCBI. Nucleic Acids Res. 2014. Pii: gku1055.)
² Sequence available on the world wide web internet site “ncbi.nlm.nih.gov/protein/”

In some embodiments, the circular polyribonucleotide comprises one or more expression sequences (coding sequences) and is configured for persistent expression in a cell of a subject in vivo. In some embodiments, the circular polyribonucleotide is configured such that expression of the one or more expression sequences in the cell at a later time point is equal to or higher than an earlier time point. In such embodiments, the expression of the one or more expression sequences may be either maintained at a relatively stable level or may increase over time. The expression of the expression sequences may be relatively stable for an extended period of time. For instance, in some cases, the expression of the one or more expression sequences in the cell over a time period of at least 7, 8, 9, 10, 12, 14, 16, 18, 20, 22, 23 or more days does not decrease by 50%, 45%, 40%, 35%, 30%, 25%, 20%, 15%, 10%, or 5%. In some cases, in some cases, the expression of the one or more expression sequences in the cell is maintained at a level that does not vary by more than 50%, 45%, 40%, 35%, 30%, 25%, 20%, 15%, 10%, or 5% for at least 7, 8, 9, 10, 12, 14, 16, 18, 20, 22, 23 or more days.

### Plant-Modifying Polypeptides

In some embodiments, the polyribonucleotide described herein (e.g., the polyribonucleotide cargo of the polyribonucleotide) includes at least one expression sequence encoding a plant-modifying polypeptide. A plant-modifying polypeptide refers to a polypeptide that can alter the genetic properties (e.g., increase gene expression, decrease gene expression, or otherwise alter the nucleotide sequence of DNA or RNA), epigenetic properties, or physiological or biochemical properties of a plant in a manner that results in a change in the plant's physiology or phenotype, e.g., an increase or decrease in the plant's fitness. In some embodiments, the polyribonucleotide encodes two, three, four, five, six, seven, eight, nine, ten or more different plant-modifying polypeptides, or multiple copies of one or more plant-modifying polypeptides. A plant-modifying polypeptide may change the physiology or phenotype of, or increase or decrease the fitness of, a variety of plants, or can be one that effects such change(s) in one or more specific plants (e.g., a specific species or genera of plants).

Examples of polypeptides that can be used herein can include an enzyme (e.g., a metabolic recombinase, a helicase, an integrase, a RNAse, a DNAse, or a ubiquitination protein), a pore-forming protein, a signaling ligand, a cell penetrating peptide, a transcription factor, a receptor, an antibody, a nanobody, a gene editing protein (e.g., CRISPR-Cas endonuclease, TALEN, or zinc finger), riboprotein, a protein aptamer, or a chaperone.

### Agricultural Polypeptides

In some embodiments, the polyribonucleotide described herein (e.g., the polyribonucleotide cargo of the polyribonucleotide) includes at least one expression sequence encoding an agricultural polypeptide. An agricultural polypeptide is a polypeptide that is suitable for an agricultural use. In embodiments, an agricultural polypeptide is applied to a plant or seed (e.g., by foliar spray, dusting, injection, or seed coating) or to the plant's environment (e.g., by soil drench or granular soil application), resulting in an alteration of the plant's physiology, phenotype, or fitness. Embodiments of an agricultural polypeptide include polypeptides that alter a level, activity, or metabolism of one or more microorganisms resident in or on a plant or non-human animal host, the alteration resulting in an increase in the host's fitness. In some embodiments the agricultural polypeptide is a plant polypeptide. In some embodiments, the agricultural polypeptide is an insect polypeptide. In some embodiments, the agricultural polypeptide has a biological effect when contacted with a non-human vertebrate animal, invertebrate animal, microbial, or plant cell.

In some embodiments, the polyribonucleotide encodes two, three, four, five, six, seven, eight, nine, ten or more agricultural polypeptides, or multiple copies of one or more agricultural polypeptides.

Embodiments of polypeptides useful in agricultural applications include, for example, bacteriocins, lysins, antimicrobial peptides, nodule C-rich peptides, and bacteriocyte regulatory peptides. Such polypeptides can be used to alter the level, activity, or metabolism of target microorganisms for increasing the fitness of insects, such as honeybees and silkworms. Embodiments of agriculturally useful polypeptides include peptide toxins, such as those naturally produced by entomopathogenic bacteria (e.g., Bacillus thuringiensis, Photorhabdus luminescens, Serratia entomophila, or Xenorhabdus nematophila), as is known in the art. Embodiments of agriculturally useful polypeptides include polypeptides (including small peptides such as cyclodipeptides or diketopiperazines) for controlling agriculturally important pests or pathogens, e.g., antimicrobial polypeptides or antifungal polypeptides for controlling diseases in plants, or pesticidal polypeptides (e.g., insecticidal polypeptides or nematicidal polypeptides) for controlling invertebrate pests such as insects or nematodes. Embodiments of agriculturally useful polypeptides include antibodies, nanobodies, and fragments thereof, e.g., antibody or nanobody fragments that retain at least some (e.g., at least 10%) of the specific binding activity of the intact antibody or nanobody. Embodiments of agriculturally useful polypeptides include transcription factors, e.g., plant transcription factors; see, e.g., the “AtTFDB” database listing the transcription factor families identified in the model plant Arabidopsis thaliana), publicly available at agris-knowledgebase[dot]org/AtTFDB/. Embodiments of agriculturally useful polypeptides include nucleases, for example, exonucleases or endonucleases (e.g., Cas nucleases such as Cas9 or Cas12a). Embodiments of agriculturally useful polypeptides further include cell-penetrating peptides, enzymes (e.g., amylases, cellulases, peptidases, lipases, chitinases), peptide pheromones (for example, yeast mating pheromones, invertebrate reproductive and larval signalling pheromones, see, e.g., Altstein (2004) Peptides, 25:1373-1376).

### Internal Ribosomal Entry Sites

In some embodiments, the polyribonucleotide described herein (e.g., the polyribonucleotide cargo of the polyribonucleotide) includes one or more internal ribosome entry site (IRES) elements. In some embodiments, the IRES is operably linked to one or more expression sequences (e.g., each IRES is operably linked to one or more expression sequences). In embodiments, the IRES is located between a heterologous promoter and the 5′ end of a coding sequence.

A suitable IRES element to include in a polyribonucleotide includes an RNA sequence capable of engaging a eukaryotic ribosome. In some embodiments, the IRES element is at least about 5 nt, at least about 8 nt, at least about 9 nt, at least about 10 nt, at least about 15 nt, at least about 20 nt, at least about 25 nt, at least about 30 nt, at least about 40 nt, at least about 50 nt, at least about 100 nt, at least about 200 nt, at least about 250 nt, at least about 350 nt, or at least about 500 nt.

In some embodiments, the IRES element is derived from the DNA of an organism including, but not limited to, a virus, a mammal, and a Drosophila. Such viral DNA may be derived from, but is not limited to, picornavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster.

In some embodiments, if present, the IRES sequence is an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, human poliovirus 1, Plautia stall intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Equine rhinitis virus, Ectropis obliqua picorna-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-I, Human BCL2, Human BiP, Human c-IAPI, Human c-myc, Human elF4G, Mouse NDST4L, Human LEF1, Mouse HIF1 alpha, Human n.myc, Mouse Gtx, Human p27kipl, Human PDGF2/c-sis, Human p53, Human Pim-I, Mouse Rbm3, Drosophila reaper, Canine Scamper, Drosophila Ubx, Human UNR, Mouse UtrA, Human VEGF-A, Human XIAP, Salivirus, Cosavirus, Parechovirus, Drosophila hairless, S. cerevisiae TFIID, S. cerevisiae YAP1, Human c-src, Human FGF-I, Simian picomavirus, Turnip crinkle virus, an aptamer to elF4G, Coxsackievirus B3 (CVB3) or Coxsackievirus A (CVB1/2). In yet another embodiment, the IRES is an IRES sequence of Coxsackievirus B3 (CVB3). In a further embodiment, the IRES is an IRES sequence of Encephalomyocarditis virus. In some embodiments, the IRES sequence has more than 90% sequence identify with one of the foregoing IRES sequences.

The IRES sequence may have a modified sequence in comparison to the wildtype IRES sequence. In some embodiments, when the last nucleotide of the wild-type IRES is not a cytosine nucleic acid residue, the last nucleotide of the wild-type IRES sequence may be modified such that it is a cytosine residue. For example, the IRES sequence may be a CVB3 IRES sequence wherein the terminal adenosine residue is modified to cytosine residue. In some embodiments, the modified CVB3 IRES may have the nucleic acid sequence of:

(SEQ ID NO: 44)
TTAAAACAGCCTGTGGGTTGATCCCACCCACAGGCCCATTGGGCGCTAG
CACTCTGGTATCACGGTACCTTTGTGCGCCTGTTTTATACCCCCTCCCC
CAACTGTAACTTAGAAGTAACACACACCGATCAACAGTCAGCGTGGCAC
ACCAGCCACGTTTTGATCAAGCACTTCTGTTACCCCGGACTGAGTATCA
ATAGACTGCTCACGCGGTTGAAGGAGAAAGCGTTCGTTATCCGGCCAAC
TACTTCGAAAAACCTAGTAACACCGTGGAAGTTGCAGAGTGTTTCGCTC
AGCACTACCCCAGTGTAGATCAGGTCGATGAGTCACCGCATTCCCCACG
GGCGACCGTGGCGGTGGCTGCGTTGGCGGCCTGCCCATGGGGAAACCCA
TGGGACGCTCTAATACAGACATGGTGCGAAGAGTCTATTGAGCTAGTTG
GTAGTCCTCCGGCCCCTGAATGCGGCTAATCCTAACTGCGGAGCACACA
CCCTCAAGCCAGAGGGCAGTGTGTCGTAACGGGCAACTCTGCAGCGGAA
CCGACTACTTTGGGTGTCCGTGTTTCATTTTATTCCTATACTGGCTGCT
TATGGTGACAATTGAGAGATCGTTACCATATAGCTATTGGATTGGCCAT
CCGGTGACTAATAGAGCTATTATATATCCCTTTGTTGGGTTTATACCAC
TTAGCTTGAAAGAGGTTAAAACATTACAATTCATTGTTAAGTTGAATAC
AGCAAC.

In some embodiments, the IRES sequence is an Enterovirus 71 (EV17) IRES. In some embodiments, the terminal guanosine residue of the EV17 IRES sequence is modified to a cytosine residue. In some embodiments, the modified EV71 IRES may have the nucleic acid sequence of:

(SEQ ID NO: 45)
ACGTTACTGGCCGAAGCCGCTTGGAATAAGGCCGGTGTGCGTTTGTCTA
TATGTTATTTTCCACCATATTGCCGTCTTTTGGCAATGTGAGGGCCCGG
AAACCTGGCCCTGTCTTCTTGACGAGCATTCCTAGGGGTCTTTCCCCTC
TCGCCAAAGGAATGCAAGGTCTGTTGAATGTCGTGAAGGAAGCAGTTCC
TCTGGAAGCTTCTTGAAGACAAACAACGTCTGTAGCGACCCTTTGCAGG
CAGCGGAACCCCCCACCTGGCGACAGGTGCCTCTGCGGCCAAAAGCCAC
GTGTATAAGATACACCTGCAAAGGCGGCACAACCCCAGTGCCACGTTGT
GAGTTGGATAGTTGTGGAAAGAGTCAAATGGCTCTCCTCAAGCGTATTC
AACAAGGGGCTGAAGGATGCCCAGAAGGTACCCCATTGTATGGGATCTG
ATCTGGGGCCTCGGTGCACATGCTTTACATGTGTTTAGTCGAGGTTAAA
AAACGTCTAGGCCCCCCGAACCACGGGGACGTGGTTTTCCTTTGAAAAA
CACGATGATAATA.

In some embodiments, the polyribonucleotide includes at least one IRES flanking at least one (e.g., 2, 3, 4, 5 or more) expression sequence. In some embodiments, the IRES flanks both sides of at least one (e.g., 2, 3, 4, 5 or more) expression sequence. In some embodiments, the polyribonucleotide includes one or more IRES sequences on one or both sides of each expression sequence, leading to separation of the resulting peptide(s) and or polypeptide(s).

In some embodiments, the polyribonucleotide cargo includes an IRES. For example, the polyribonucleotide cargo may include a circular RNA IRES, e.g., as described in Fan et al. Nature Communications 13(1): 3751, 2022 doi: 10.1038/s41467-022-31327-y; Chen et al. Mol. Cell 81:1-19, 2021; Jopling et al. Oncogene 20:2664-2670, 2001; Baranick et al. PNAS 105 (12): 4733-4738, 2008; Lang et al. Molecular Biology of the Cell 13 (5): 1792-1801, 2002; Dorokhov et al. PNAS 99 (8): 5301-5306, 2002; Wang et al. Nucleic Acids Research 33 (7): 2248-2258, 2005; Petz et a. Nucleic Acids Research 35(8): 2473-2482, 2007; and International Patent Publication No. WO 2021/263124 A2, each of which is hereby incorporated by reference in its entirety.

### Regulatory Elements

In some embodiments, the polyribonucleotide described herein (e.g., the polyribonucleotide cargo of the polyribonucleotide) includes one or more regulatory elements. In some embodiments, the polyribonucleotide includes a regulatory element, e.g., a sequence that modifies expression of an expression sequence within the polyribonucleotide.

A regulatory element may include a sequence that is located adjacent to an expression sequence that encodes an expression product. A regulatory element may be linked operatively to the adjacent sequence. A regulatory element may increase an amount of product expressed as compared to an amount of the expressed product when no regulatory element exists. In addition, one regulatory element can increase an amount of products expressed for multiple expression sequences attached in tandem. Hence, one regulatory element can enhance the expression of one or more expression sequences. Multiple regulatory elements are well-known to persons of ordinary skill in the art.

In some embodiments, the regulatory element is a translation modulator. A translation modulator can modulate translation of the expression sequence in the polyribonucleotide. A translation modulator can be a translation enhancer or suppressor. In some embodiments, the polyribonucleotide includes at least one translation modulator adjacent to at least one expression sequence. In some embodiments, the polyribonucleotide includes a translation modulator adjacent each expression sequence. In some embodiments, the translation modulator is present on one or both sides of each expression sequence, leading to separation of the expression products, e.g., peptide(s) and or polypeptide(s).

In some embodiments, the regulatory element is a microRNA (miRNA) or a miRNA binding site.

Further examples of regulatory elements are described, e.g., in paragraphs [0154]-[0161] of International Patent Publication No. WO2019/118919, which is hereby incorporated by reference in its entirety.

### Translation Initiation Sequences

In some embodiments, the polyribonucleotide described herein (e.g., the polyribonucleotide cargo of the polyribonucleotide) includes at least one translation initiation sequence. In some embodiments, the polyribonucleotide includes a translation initiation sequence operably linked to an expression sequence.

In some embodiments, the polyribonucleotide encodes a polypeptide and may include a translation initiation sequence, e.g., a start codon. In some embodiments, the translation initiation sequence includes a Kozak or Shine-Dalgamo sequence. In some embodiments, the polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the polyribonucleotide includes at least one translation initiation sequence adjacent to an expression sequence. In some embodiments, the translation initiation sequence provides conformational flexibility to the polyribonucleotide. In some embodiments, the translation initiation sequence is within a substantially single stranded region of the polyribonucleotide. Further examples of translation initiation sequences are described in paragraphs [0163]-[0165] of International Patent Publication No. WO2019/118919, which is hereby incorporated by reference in its entirety.

The polyribonucleotide may include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. Translation may initiate on the first start codon or may initiate downstream of the first start codon.

In some embodiments, the polyribonucleotide may initiate at a codon which is not the first start codon, e.g., AUG. Translation of the polyribonucleotide may initiate at an alternative translation initiation sequence, such as, but not limited to, ACG, AGG, AAG, CTG/CUG, GTG/GUG, ATA/AUA, ATT/AUU, TTG/UUG. In some embodiments, translation begins at an alternative translation initiation sequence under selective conditions, e.g., stress induced conditions. As a non-limiting example, the translation of the polyribonucleotide may begin at alternative translation initiation sequence, such as ACG. As another non-limiting example, the polyribonucleotide translation may begin at alternative translation initiation sequence, CTG/CUG. As another non-limiting example, the polyribonucleotide translation may begin at alternative translation initiation sequence, GTG/GUG. As another non-limiting example, the polyribonucleotide may begin translation at a repeat-associated non-AUG (RAN) sequence, such as an alternative translation initiation sequence that includes short stretches of repetitive RNA e.g., CGG, GGGGCC, CAG, CTG.

### Termination Elements

In some embodiments, the polyribonucleotide described herein (e.g., the polyribonucleotide cargo of the polyribonucleotide) includes least one termination element. In some embodiments, the polyribonucleotide includes a termination element operably linked to an expression sequence. In some embodiments, the polynucleotide lacks a termination element.

In some embodiments, the polyribonucleotide includes one or more expression sequences, and each expression sequence may or may not have a termination element. In some embodiments, the polyribonucleotide includes one or more expression sequences, and the expression sequences lack a termination element, such that the polyribonucleotide is continuously translated. Exclusion of a termination element may result in rolling circle translation or continuous expression of expression product.

In some embodiments, the circular polyribonucleotide includes one or more expression sequences, and each expression sequence may or may not have a termination element. In some embodiments, the circular polyribonucleotide includes one or more expression sequences, and the expression sequences lack a termination element, such that the circular polyribonucleotide is continuously translated. Exclusion of a termination element may result in rolling circle translation or continuous expression of expression product, e.g., peptides or polypeptides, due to lack of ribosome stalling or fall-off. In such an embodiment, rolling circle translation expresses a continuous expression product through each expression sequence. In some other embodiments, a termination element of an expression sequence can be part of a stagger element. In some embodiments, one or more expression sequences in the circular polyribonucleotide comprises a termination element. However, rolling circle translation or expression of a succeeding (e.g., second, third, fourth, fifth, etc.) expression sequence in the circular polyribonucleotide is performed. In such instances, the expression product may fall off the ribosome when the ribosome encounters the termination element, e.g., a stop codon, and terminates translation. In some embodiments, translation is terminated while the ribosome, e.g., at least one subunit of the ribosome, remains in contact with the circular polyribonucleotide.

In some embodiments, the circular polyribonucleotide includes a termination element at the end of one or more expression sequences. In some embodiments, one or more expression sequences comprises two or more termination elements in succession. In such embodiments, translation is terminated and rolling circle translation is terminated. In some embodiments, the ribosome completely disengages with the circular polyribonucleotide. In some such embodiments, production of a succeeding (e.g., second, third, fourth, fifth, etc.) expression sequence in the circular polyribonucleotide may require the ribosome to reengage with the circular polyribonucleotide prior to initiation of translation. Generally, termination elements include an in-frame nucleotide triplet that signals termination of translation, e.g., UAA, UGA, UAG. In some embodiments, one or more termination elements in the circularpolyribonucleotide are frame-shifted termination elements, such as but not limited to, off-frame or −1 and +1 shifted reading frames (e.g., hidden stop) that may terminate translation. Frame-shifted termination elements include nucleotide triples, TAA, TAG, and TGA that appear in the second and third reading frames of an expression sequence. Frame-shifted termination elements may be important in preventing misreads of mRNA, which is often detrimental to the cell. In some embodiments, the termination element is a stop codon.

Further examples of termination elements are described in paragraphs [0169]-[0170] of International Patent Publication No. WO2019/118919, which is hereby incorporated by reference in its entirety.

### Untranslated Regions

In some embodiments, a circular polyribonucleotide includes untranslated regions (UTRs). UTRs of a genomic region including a gene may be transcribed but not translated. In some embodiments, a UTR may be included upstream of the translation initiation sequence of an expression sequence described herein. In some embodiments, a UTR may be included downstream of an expression sequence described herein. In some instances, one UTR for first expression sequence is the same as or continuous with or overlapping with another UTR for a second expression sequence. In some embodiments, the intron is a human intron. In some embodiments, the intron is a full-length human intron, e.g., ZKSCAN1.

Exemplary untranslated regions are described in paragraphs [0197]-[201] of International Patent Publication No. WO2019/118919, which is hereby incorporated by reference in its entirety.

In some embodiments, a circular polyribonucleotide includes a poly-A sequence. Exemplary poly-A sequences are described in paragraphs [0202]-[0205] of International Patent Publication No. WO2019/118919, which is hereby incorporated by reference in its entirety. In some embodiments, a circular polyribonucleotide lacks a poly-A sequence.

In some embodiments, a circular polyribonucleotide includes a UTR with one or more stretches of Adenosines and Uridines embedded within. These AU rich signatures may increase turnover rates of the expression product.

Introduction, removal, or modification of UTR AU rich elements (AREs) may be useful to modulate the stability, or immunogenicity (e.g., the level of one or more marker of an immune or inflammatory response) of the circular polyribonucleotide. When engineering specific circular polyribonucleotides, one or more copies of an ARE may be introduced to the circular polyribonucleotide and the copies of an ARE may modulate translation and/or production of an expression product. Likewise, AREs may be identified and removed or engineered into the circular polyribonucleotide to modulate the intracellular stability and thus affect translation and production of the resultant protein.

It should be understood that any UTR from any gene may be incorporated into the respective flanking regions of the circular polyribonucleotide.

In some embodiments, a circular polyribonucleotide lacks a 5′-UTR and is competent for protein expression from its one or more expression sequences. In some embodiments, the circular polyribonucleotide lacks a 3′-UTR and is competent for protein expression from its one or more expression sequences. In some embodiments, the circular polyribonucleotide lacks a poly-A sequence and is competent for protein expression from its one or more expression sequences. In some embodiments, the circular polyribonucleotide lacks a termination element and is competent for protein expression from its one or more expression sequences. In some embodiments, the circular polyribonucleotide lacks an internal ribosomal entry site and is competent for protein expression from its one or more expression sequences. In some embodiments, the circular polyribonucleotide lacks a cap and is competent for protein expression from its one or more expression sequences. In some embodiments, the circular polyribonucleotide lacks a 5′-UTR, a 3′-UTR, and an IRES, and is competent for protein expression from its one or more expression sequences. In some embodiments, the circular polyribonucleotide includes one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (e.g., siRNA, lncRNAs, shRNA), and a sequence that encodes a therapeutic mRNA or protein.

In some embodiments, a circular polyribonucleotide lacks a 5′-UTR. In some embodiments, the circular polyribonucleotide lacks a 3′-UTR. In some embodiments, the circular polyribonucleotide lacks a poly-A sequence. In some embodiments, the circular polyribonucleotide lacks a termination element. In some embodiments, the circular polyribonucleotide lacks an internal ribosomal entry site. In some embodiments, the circular polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent, e.g., that is comparable to or similar to in the absence of exonuclease. In some embodiments, the circular polyribonucleotide is not degraded by exonucleases. In some embodiments, the circular polyribonucleotide has reduced degradation when exposed to exonuclease. In some embodiments, the circular polyribonucleotide lacks binding to a cap-binding protein. In some embodiments, the circular polyribonucleotide lacks a 5′ cap.

### Stagger Elements

In some embodiments, the circular polyribonucleotide includes at least one stagger element adjacent to an expression sequence. In some embodiments, the circular polyribonucleotide includes a stagger element adjacent to each expression sequence. In some embodiments, the stagger element is present on one or both sides of each expression sequence, leading to separation of the expression products, e.g., peptide(s) and or polypeptide(s). In some embodiments, the stagger element is a portion of the one or more expression sequences. In some embodiments, the circular polyribonucleotide comprises one or more expression sequences, and each of the one or more expression sequences is separated from a succeeding expression sequence by a stagger element on the circular polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element comprises a portion of an expression sequence of the one or more expression sequences.

In some embodiments, the circular polyribonucleotide includes a stagger element. To avoid production of a continuous expression product, e.g., peptide or polypeptide, while maintaining rolling circle translation, a stagger element may be included to induce ribosomal pausing during translation. In some embodiments, the stagger element is at 3′ end of at least one of the one or more expression sequences. The stagger element can be configured to stall a ribosome during rolling circle translation of the circular polyribonucleotide. The stagger element may include, but is not limited to a 2A-like, or CHYSEL (SEQ ID NO: 17) (cis-acting hydrolase element) sequence. In some embodiments, the stagger element encodes a sequence with a C-terminal consensus sequence that is X₁X₂X₃EX₅NPGP (SEQ ID NO: 18), where X₁ is absent or G or H, X₂ is absent or D or G, X₃ is D or V or I or S or M, and X₅ is any amino acid. In some embodiments, this sequence comprises a non-conserved sequence of amino-acids with a strong alpha-helical propensity followed by the consensus sequence -D(V/I)EXNPGP (SEQ ID NO: 19), where x=any amino acid. Some nonlimiting examples of stagger elements includes GDVESNPGP (SEQ ID NO: 20), GDIEENPGP (SEQ ID NO: 21), VEPNPGP (SEQ ID NO: 22), IETNPGP (SEQ ID NO: 23), GDIESNPGP (SEQ ID NO: 24), GDVELNPGP (SEQ ID NO: 25), GDIETNPGP (SEQ ID NO: 26), GDVENPGP (SEQ ID NO: 27), GDVEENPGP (SEQ ID NO: 28), GDVEQNPGP (SEQ ID NO: 29), IESNPGP (SEQ ID NO: 30), GDIELNPGP (SEQ ID NO: 31), HDIETNPGP (SEQ ID NO: 32), HDVETNPGP (SEQ ID NO: 33), HDVEMNPGP (SEQ ID NO: 34), GDMESNPGP (SEQ ID NO: 35), GDVETNPGP (SEQ ID NO: 36) GDIEQNPGP (SEQ ID NO: 37), and DSEFNPGP (SEQ ID NO: 38).

In some embodiments, the stagger element described herein cleaves an expression product, such as between G and P of the consensus sequence described herein. As one non-limiting example, the circular polyribonucleotide includes at least one stagger element to cleave the expression product. In some embodiments, the circular polyribonucleotide includes a stagger element adjacent to at least one expression sequence. In some embodiments, the circular polyribonucleotide includes a stagger element after each expression sequence. In some embodiments, the circular polyribonucleotide includes a stagger element is present on one or both sides of each expression sequence, leading to translation of individual peptide(s) and or polypeptide(s) from each expression sequence.

In some embodiments, a stagger element comprises one or more modified nucleotides or unnatural nucleotides that induce ribosomal pausing during translation. Unnatural nucleotides may include peptide nucleic acid (PNA), Morpholino and locked nucleic acid (LNA), as well as glycol nucleic acid (GNA) and threose nucleic acid (TNA). Examples such as these are distinguished from naturally occurring DNA or RNA by changes to the backbone of the molecule. Exemplary modifications can include any modification to the sugar, the nucleobase, the internucleoside linkage (e.g., to a linking phosphate/to a phosphodiester linkage/to the phosphodiester backbone), and any combination thereof that can induce ribosomal pausing during translation. Some of the exemplary modifications provided herein are described elsewhere herein.

In some embodiments, the stagger element is present in the circular polyribonucleotide in other forms. For example, in some exemplary circular polyribonucleotides, a stagger element comprises a termination element of a first expression sequence in the circular polyribonucleotide, and a nucleotide spacer sequence that separates the termination element from a first translation initiation sequence of an expression succeeding the first expression sequence. In some examples, the first stagger element of the first expression sequence is upstream of (5′ to) a first translation initiation sequence of the expression succeeding the first expression sequence in the circular polyribonucleotide. In some cases, the first expression sequence and the expression sequence succeeding the first expression sequence are two separate expression sequences in the circular polyribonucleotide. The distance between the first stagger element and the first translation initiation sequence can enable continuous translation of the first expression sequence and its succeeding expression sequence.

In some embodiments, the first stagger element comprises a termination element and separates an expression product of the first expression sequence from an expression product of its succeeding expression sequences, thereby creating discrete expression products. In some cases, the circular polyribonucleotide comprising the first stagger element upstream of the first translation initiation sequence of the succeeding sequence in the circular polyribonucleotide is continuously translated, while a corresponding circular polyribonucleotide comprising a stagger element of a second expression sequence that is upstream of a second translation initiation sequence of an expression sequence succeeding the second expression sequence is not continuously translated. In some cases, there is only one expression sequence in the circular polyribonucleotide, and the first expression sequence and its succeeding expression sequence are the same expression sequence. In some exemplary circular polyribonucleotides, a stagger element comprises a first termination element of a first expression sequence in the circular polyribonucleotide, and a nucleotide spacer sequence that separates the termination element from a downstream translation initiation sequence. In some such examples, the first stagger element is upstream of (5′ to) a first translation initiation sequence of the first expression sequence in the circular polyribonucleotide. In some cases, the distance between the first stagger element and the first translation initiation sequence enables continuous translation of the first expression sequence and any succeeding expression sequences.

In some embodiments, the first stagger element separates one round expression product of the first expression sequence from the next round expression product of the first expression sequences, thereby creating discrete expression products. In some cases, the circular polyribonucleotide comprising the first stagger element upstream of the first translation initiation sequence of the first expression sequence in the circular polyribonucleotide is continuously translated, while a corresponding circular polyribonucleotide comprising a stagger element upstream of a second translation initiation sequence of a second expression sequence in the corresponding circular polyribonucleotide is not continuously translated. In some cases, the distance between the second stagger element and the second translation initiation sequence is at least 2×, 3×, 4×, 5×, 6×, 7×, 8×, 9×, or 10× greater in the corresponding circular polyribonucleotide than a distance between the first stagger element and the first translation initiation in the circular polyribonucleotide. In some cases, the distance between the first stagger element and the first translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater. In some embodiments, the distance between the second stagger element and the second translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater than the distance between the first stagger element and the first translation initiation. In some embodiments, the circular polyribonucleotide comprises more than one expression sequence. Examples of stagger elements are described in paragraphs [0172]-[0175] of International Patent Publication No. WO2019/118919, which is hereby incorporated by reference in its entirety.

### Non-Coding Sequences

In some embodiments, the polyribonucleotide described herein (e.g., the polyribonucleotide cargo of the polyribonucleotide) includes one or more non-coding sequence, e.g., a sequence that does not encode the expression of polypeptide. In some embodiments, the polyribonucleotide includes two, three, four, five, six, seven, eight, nine, ten or more than ten non-coding sequences. In some embodiments, the polyribonucleotide does not encode a polypeptide expression sequence.

Noncoding sequences can be natural or synthetic sequences. In some embodiments, a noncoding sequence can alter cellular behavior, such as e.g., lymphocyte behavior. In some embodiments, the noncoding sequences are antisense to cellular RNA sequences.

In some embodiments, the polyribonucleotide includes regulatory nucleic acids that are RNA or RNA-like structures typically from about 5-500 base pairs (bp) (depending on the specific RNA structure (e.g., miRNA 5-30 bp, lncRNA 200-500 bp) and may have a nucleobase sequence identical (complementary) or nearly identical (substantially complementary) to a coding sequence in an expressed target gene within the cell. In embodiments, the circular polyribonucleotide includes regulatory nucleic acids that encode an RNA precursor that can be processed to a smaller RNA, e.g., a miRNA precursor, which can be from about 50 to about 1000 bp, that can be processed to a smaller miRNA intermediate or a mature miRNA.

Long non-coding RNAs (lncRNA) are defined as non-protein coding transcripts longer than 100 nucleotides. Many lncRNAs are characterized as tissue specific. Divergent lncRNAs that are transcribed in the opposite direction to nearby protein-coding genes include a significant proportion (e.g., about 20% of total lncRNAs in mammalian genomes) and possibly regulate the transcription of the nearby gene. In one embodiment, the polyribonucleotide provided herein includes a sense strand of a lncRNA. In one embodiment, the polyribonucleotide provided herein includes an antisense strand of a lncRNA.

In embodiments, the polyribonucleotide encodes a regulatory nucleic acid that is substantially complementary, or fully complementary, to all or to at least one fragment of an endogenous gene or gene product (e.g., mRNA). In embodiments, the regulatory nucleic acids complement sequences at the boundary between introns and exons, in between exons, or adjacent to an exon, to prevent the maturation of newly generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof. In some embodiments, the regulatory nucleic acid includes a protein-binding site that can bind to a protein that participates in regulation of expression of an endogenous gene or an exogenous gene.

In embodiments, the polyribonucleotide encodes a regulatory RNA that hybridizes to a transcript of interest wherein the regulatory RNA has a length of from about 5 to 30 nucleotides, from about 10 to 30 nucleotides, or about 11, 12, 13, 14, 15, 16, 17, 18, 19, 20, 21, 22, 23, 24, 25, 26, 27, 28, 29, 30 or more than 30 nucleotides. In embodiments, the degree of sequence identity of the regulatory RNA to the targeted transcript is at least 75%, at least 80%, at least 85%, at least 90%, or at least 95%.

In embodiments, the polyribonucleotide encodes a microRNA (miRNA) molecule identical to about 5 to about 25 contiguous nucleotides of a target gene or encodes a precursor to that miRNA. In some embodiments, the miRNA has a sequence that allows the mRNA to recognize and bind to a specific target mRNA. In embodiments, the miRNA sequence commences with the dinucleotide AA, includes a GC-content of about 30-70% (about 30-60%, about 40-60%, or about 45%-55%), and does not have a high percentage identity to any nucleotide sequence other than the target in the genome of the subject (e.g., a mammal) in which it is to be introduced, for example as determined by standard BLAST search.

In some embodiments, the polyribonucleotide includes at least one miRNA (or miRNA precursor), e.g., 2, 3, 4, 5, 6, or more miRNAs or miRNA precursors. In some embodiments, the polyribonucleotide includes a sequence that encodes a miRNA (or its precursor) having at least about 75%, 80%, 85%, 90% 95%, 96%, 97%, 98%, or 99% or 100% nucleotide sequence complementarity to a target sequence.

siRNAs and shRNAs resemble intermediates in the processing pathway of the endogenous microRNA (miRNA) genes. In some embodiments, siRNAs can function as miRNAs and vice versa. MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation. Known miRNA binding sites are within mRNA 3′ UTRs; miRNAs seem to target sites with near-perfect complementarity to nucleotides 2-8 from the miRNA's 5′ end. This region is known as the seed region. Because mature siRNAs and miRNAs are interchangeable, exogenous siRNAs downregulate mRNAs with seed complementarity to the siRNA.

Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs.

### Protein-Binding Sequences

In some embodiments, a circular polyribonucleotide includes one or more protein binding sites that enable a protein, e.g., a ribosome, to bind to an internal site in the RNA sequence. By engineering protein binding sites, e.g., ribosome binding sites, into the circular polyribonucleotide, the circular polyribonucleotide may evade or have reduced detection by the host's immune system, have modulated degradation, or modulated translation, by masking the circular polyribonucleotide from components of the host's immune system.

In some embodiments, a circular polyribonucleotide includes at least one immunoprotein binding site, for example to evade immune responses, e.g., CTL (cytotoxic T lymphocyte) responses. In some embodiments, the immunoprotein binding site is a nucleotide sequence that binds to an immunoprotein and aids in masking the circular polyribonucleotide as exogenous. In some embodiments, the immunoprotein binding site is a nucleotide sequence that binds to an immunoprotein and aids in hiding the circular polyribonucleotide as exogenous or foreign.

Traditional mechanisms of ribosome engagement to linear RNA involve ribosome binding to the capped 5′ end of an RNA. From the 5′ end, the ribosome migrates to an initiation codon, whereupon the first peptide bond is formed. According to the present disclosure, internal initiation (i.e., cap-independent) of translation of the circular polyribonucleotide does not require a free end or a capped end. Rather, a ribosome binds to a non-capped internal site, whereby the ribosome begins polypeptide elongation at an initiation codon. In some embodiments, the circular polyribonucleotide includes one or more RNA sequences including a ribosome binding site, e.g., an initiation codon.

Natural 5′UTRs bear features which play roles in for translation initiation. They harbor signatures like Kozak sequences which are commonly known to be involved in the process by which the ribosome initiates translation of many genes. Kozak sequences have the consensus CCR (A/G) CCAUGG (SEQ ID NO: 39), where R is a purine (adenine or guanine) three bases upstream of the start codon (AUG), which is followed by another ‘G’. 5′ UTR also have been known to form secondary structures which are involved in elongation factor binding.

In some embodiments, a circular polyribonucleotide encodes a protein binding sequence that binds to a protein. In some embodiments, the protein binding sequence targets or localizes the circular polyribonucleotide to a specific target. In some embodiments, the protein binding sequence specifically binds an arginine-rich region of a protein.

In some embodiments, the protein binding site includes, but is not limited to, a binding site to the protein such as ACIN1, AGO, APOBEC3F, APOBEC3G, ATXN2, AUH, BCCIP, CAPRIN1, CELF2, CPSF1, CPSF2, CPSF6, CPSF7, CSTF2, CSTF2T, CTCF, DDX21, DDX3, DDX3X, DDX42, DGCR8, EIF3A, EIF4A3, EIF4G2, ELAVL1, ELAVL3, FAM120A, FBL, FIP1L1, FKBP4, FMR1, FUS, FXR1, FXR2, GNL3, GTF2F1, HNRNPA1, HNRNPA2B1, HNRNPC, HNRNPK, HNRNPL, HNRNPM, HNRNPU, HNRNPUL1, IGF2BP1, IGF2BP2, IGF2BP3, ILF3, KHDRBS1, LARP7, LIN28A, LIN28B, m6A, MBNL2, METTL3, MOV10, MSI1, MSI2, NONO, NONO-, NOP58, NPM1, NUDT21, PCBP2, POLR2A, PRPF8, PTBP1, RBFOX2, RBM10, RBM22, RBM27, RBM47, RNPS1, SAFB2, SBDS, SF3A3, SF3B4, SIRT7, SLBP, SLTM, SMNDC1, SND1, SRRM4, SRSF1, SRSF3, SRSF7, SRSF9, TAF15, TARDBP, TIA1, TNRC6A, TOP3B, TRA2A, TRA2B, U2AF1, U2AF2, UNK, UPF1, WDR33, XRN2, YBX1, YTHDC1, YTHDF1, YTHDF2, YWHAG, ZC3H7B, PDK1, AKT1, and any other protein that binds RNA.

### Spacer Sequences

In some embodiments, the polyribonucleotide described herein includes one or more spacer sequences. A spacer refers to any contiguous nucleotide sequence (e.g., of one or more nucleotides) that provides distance or flexibility between two adjacent polynucleotide regions. Spacers may be present in between any of the nucleic acid elements described herein. Spacers may also be present within a nucleic acid element described herein.

For example, wherein a nucleic acid includes any two or more of the following elements: (A) a 3′ half of Group I catalytic intron fragment; (B) a 3′ splice site; (C) a 3′ exon fragment; (D) a polyribonucleotide cargo; (E) a 5′ exon fragment; (F) a 5′ splice site; and (G) a 5′ half of Group I catalytic intron fragment; a spacer region may be present between any one or more of the elements. Any of elements (A), (B), (C), (D), (E), (F), or (G) may be separated by a spacer sequence, as described herein. For example, there may be a spacer between (A) and (B), between (B) and (C), between (C) and (D), between (D) and (E), between (E) and (F), or between (F) and (G).

In some embodiments, the polyribonucleotide further includes a first spacer region between the 5′ exon fragment of (C) and the polyribonucleotide cargo of (D). The spacer may be, e.g., at least 5 (e.g., at least 10, at least 15, at least 20) ribonucleotides in length. In some embodiments, the polyribonucleotide further includes a second spacer region between the polyribonucleotide cargo of (D) and the 5′ exon fragment of (E). The spacer may be, e.g., at least 5 (e.g., at least 10, at least 15, at least 20) ribonucleotides in length. In some embodiments, each spacer region is at least 5 (e.g., at least 10, at least 15, at least 20) ribonucleotides in length. Each spacer region may be, e.g., from 5 to 500 (e.g., 10, 20, 30, 40, 50, 60, 70, 80, 90, 100, 150, 200, 250, 300, 350, 400, 450, or 500) ribonucleotides in length. The first spacer region, the second spacer region, or the first spacer region and the second spacer region may include a polyA sequence. The first spacer region, the second spacer region, or the first spacer region and the second spacer region may include a polyA-C sequence. In some embodiments, the first spacer region, the second spacer region, or the first spacer region and the second spacer region includes a polyA-G sequence. In some embodiments, the first spacer region, the second spacer region, or the first spacer region and the second spacer region includes a polyA-T sequence. In some embodiments, the first spacer region, the second spacer region, or the first spacer region and the second spacer region includes a random sequence.

Spacers may also be present within a nucleic acid region described herein. For example, a polynucleotide cargo region may include one or multiple spacers. Spacers may separate regions within the polynucleotide cargo.

In some embodiments, the spacer sequence can be, for example, at least 10 nucleotides in length, at least 15 nucleotides in length, or at least 30 nucleotides in length. In some embodiments, the spacer sequence is at least 7, 8, 9, 10, 11, 12, 13, 14, 15, 16, 17, 18, 19, 20, 25 or 30 nucleotides in length. In some embodiments, the spacer sequence is no more than 100, 90, 80, 70, 60, 50, 45, 40, 35 or 30 nucleotides in length. In some embodiments the spacer sequence is from 20 to 50 nucleotides in length. In certain embodiments, the spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length.

The spacer sequences can be polyA sequences, polyA-C sequences, polyC sequences, or poly-U sequences.

In some embodiments, the spacer sequences can be polyA-T, polyA-U, polyA-C, polyA-G, or a random sequence. For example, the spacer sequence may consist of a polyA region having 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) adenosine residues. The spacer sequence may consist of a polyA-C region having from 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) adenosine or cytosine residues. In some embodiments, the spacer sequence may consist of a polyA-U region having from 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) adenosine or uridine residues. In some embodiments, the spacer sequence may consist of a polyA-G region having from 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) adenosine or guanosine residues. In some embodiments, the spacer sequence may consist of a polyA-T region having from 80% to 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) adenosine or thymine residues.

A spacer sequence may be used to separate an IRES from adjacent structural elements to martini the structure and function of the IRES or the adjacent element. A spacer can be specifically engineered depending on the IRES. In some embodiments, an RNA folding computer software, such as RNAFold, can be utilized to guide designs of the various elements of the vector, including the spacers.

In some embodiments, the polyribonucleotide includes a 5′ spacer sequence (e.g., between the 5′ annealing region and the polyribonucleotide cargo). In some embodiments, the 5′ spacer sequence is at least 10 nucleotides in length. In another embodiment, the 5′ spacer sequence is at least 15 nucleotides in length. In a further embodiment, the 5′ spacer sequence is at least 30 nucleotides in length. In some embodiments, the 5′ spacer sequence is at least 7, 8, 9, 10, 11, 12, 13, 14, 15, 16, 17, 18, 19, 20, 25 or 30 nucleotides in length. In some embodiments, the 5′ spacer sequence is no more than 100, 90, 80, 70, 60, 50, 45, 40, 35 or 30 nucleotides in length. In some embodiments the 5′ spacer sequence is between 20 and 50 nucleotides in length. In certain embodiments, the 5′ spacer sequence is 10, 11, 12, 13, 14, 15, 16, 17, 18, 19, 20, 21, 22, 23, 24, 25, 26, 27, 28, 29, 30, 31, 32, 33, 34, 35, 36 37, 38, 39, 40, 41, 42, 43, 44, 45, 46, 47, 48, 49 or 50 nucleotides in length. In one embodiment, the 5′ spacer sequence is a polyA sequence. In another embodiment, the 5′ spacer sequence is a polyA-C sequence. In some embodiments, the 5′ spacer sequence includes a polyA-G sequence. In some embodiments, the 5′ spacer sequence includes a polyA-T sequence. In some embodiments, the 5′ spacer sequence includes a random sequence.

In some embodiments, the polyribonucleotide includes a 3′ spacer sequence (e.g., between the 3′ annealing region and the polyribonucleotide cargo). In some embodiments, the 3′ spacer sequence is at least 10 nucleotides in length. In another embodiment, the 3′ spacer sequence is at least 15 nucleotides in length. In a further embodiment, the 3′ spacer sequence is at least 30 nucleotides in length. In some embodiments, the 3′ spacer sequence is at least 7, 8, 9, 10, 11, 12, 13, 14, 15, 16, 17, 18, 19, 20, 25 or 30 nucleotides in length. In some embodiments, the 3′ spacer sequence is no more than 100, 90, 80, 70, 60, 50, 45, 40, 35 or 30 nucleotides in length. In some embodiments the 3′ spacer sequence is from 20 to 50 nucleotides in length. In certain embodiments, the 3′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In one embodiment, the 3′ spacer sequence is a polyA sequence. In another embodiment, the 3′ spacer sequence is a polyA-C sequence. In some embodiments, the 3′ spacer sequence includes a polyA-G sequence. In some embodiments, the 3′ spacer sequence includes a polyA-T sequence. In some embodiments, the 3′ spacer sequence includes a random sequence.

In one embodiment, the polyribonucleotide includes a 5′ spacer sequence, but not a 3′ spacer sequence. In another embodiment, the polyribonucleotide includes a 3′ spacer sequence, but not a 5′ spacer sequence. In another embodiment, the polyribonucleotide includes neither a 5′ spacer sequence, nor a 3′ spacer sequence. In another embodiment, the polyribonucleotide does not include an IRES sequence. In a further embodiment, the polyribonucleotide does not include an IRES sequence, a 5′ spacer sequence or a 3′ spacer sequence.

In some embodiments, the spacer sequence includes at least 3 ribonucleotides, at least 4 ribonucleotides, at least 5 ribonucleotides, at least about 8 ribonucleotides, at least about 10 ribonucleotides, at least about 12 ribonucleotides, at least about 15 ribonucleotides, at least about 20 ribonucleotides, at least about 25 ribonucleotides, at least about 30 ribonucleotides, at least about 40 ribonucleotides, at least about 50 ribonucleotides, at least about 60 ribonucleotides, at least about 70 ribonucleotides, at least about 80 ribonucleotides, at least about 90 ribonucleotides, at least about 100 ribonucleotides, at least about 120 ribonucleotides, at least about 150 ribonucleotides, at least about 200 ribonucleotides, at least about 250 ribonucleotides, at least about 300 ribonucleotides, at least about 400 ribonucleotides, at least about 500 ribonucleotides, at least about 600 ribonucleotides, at least about 700 ribonucleotides, at least about 800 ribonucleotides, at least about 900 ribonucleotides, or at least about 100 ribonucleotides.

### Methods of Production

The disclosure also provides methods of producing a circular RNA. For example, a deoxyribonucleotide template may be transcribed in a cell-free system (e.g., by in vitro transcription) to a produce a linear RNA. The linear polyribonucleotide produces a splicing-compatible polyribonucleotide, which may be self-spliced to produce a circular polyribonucleotide.

### Methods of Production in a Cell-Free System

In some embodiments, the disclosure provides a method of producing a circular polyribonucleotide (e.g., in a cell-free system) by providing a linear polyribonucleotide; and self-splicing linear polyribonucleotide under conditions suitable for splicing of the 3′ and 5′ splice sites of the linear polyribonucleotide; thereby producing a circular polyribonucleotide.

In some embodiments, the disclosure provides a method of producing a circular polyribonucleotide by providing a deoxyribonucleotide encoding the linear polyribonucleotide; transcribing the deoxyribonucleotide in a cell-free system to produce the linear polyribonucleotide; optionally purifying the splicing-compatible linear polyribonucleotide; and self-splicing the linear polyribonucleotide under conditions suitable for splicing of the 3′ and 5′ splice sites of the linear polyribonucleotide, thereby producing a circular polyribonucleotide.

In some embodiments, the disclosure provides a method of producing a circular polyribonucleotide by providing a deoxyribonucleotide encoding a linear polyribonucleotide; transcribing the deoxyribonucleotide in a cell-free system to produce the linear polyribonucleotide, wherein the transcribing occurs in a solution under conditions suitable for splicing of the 3′ and 5′ splice sites of the linear polyribonucleotide, thereby producing a circular polyribonucleotide. In some embodiments, the linear polyribonucleotide comprises a 5′ split-intron and a 3′ split-intron (e.g., a self-splicing construct for producing a circular polyribonucleotide). In some embodiments, the linear polyribonucleotide comprises a 5′ annealing region and a 3′ annealing region.

Suitable conditions for in vitro transcriptions and or self-splicing may include any conditions (e.g., a solution or a buffer, such as an aqueous buffer or solution) that mimic physiological conditions in one or more respects. In some embodiments, suitable conditions include between 0.1-100 mM Mg2+ ions or a salt thereof (e.g., 1-100 mM, 1-50 mM, 1-20 mM, 5-50 mM, 5-20 mM, or 5-15 mM). In some embodiments, suitable conditions include between 1-1000 mM K+ ions or a salt thereof such as KCl (e.g., 1-1000 mM, 1-500 mM, 1-200 mM, 50-500 mM, 100-500 mM, or 100-300 mM). In some embodiments, suitable conditions include between 1-1000 mM Cl− ions or a salt thereof such as KCl (e.g., 1-1000 mM, 1-500 mM, 1-200 mM, 50-500 mM, 100-500 mM, or 100-300 mM). In some embodiments, suitable conditions include between 0.1-100 mM Mn2+ ions or a salt thereof such as MnCl2 (e.g., 0.1-100 mM, 0.1-50 mM, 0.1-20 mM, 0.1-10 mM, 0.1-5 mM, 0.1-2 mM, 0.5-50 mM, 0.5-20 mM, 0.5-15 mM, 0.5-5 mM, 0.5-2 mM, or 0.1-10 mM). In some embodiments, suitable conditions include dithiothreitol (DTT) (e.g., 1-1000 μM, 1-500 μM, 1-200 μM, 50-500 μM, 100-500 μM, 100-300 μM, 0.1-100 mM, 0.1-50 mM, 0.1-20 mM, 0.1-10 mM, 0.1-5 mM, 0.1-2 mM, 0.5-50 mM, 0.5-20 mM, 0.5-15 mM, 0.5-5 mM, 0.5-2 mM, or 0.1-10 mM). In some embodiments, suitable conditions include between 0.1 mM and 100 mM ribonucleoside triphosphate (NTP) (e.g., 0.1-100 mM, 0.1-50 mM, 0.1-10 mM, 1-100 mM, 1-50 mM, or 1-10 mM). In some embodiments, suitable conditions include a pH of 4 to 10 (e.g., pH of 5 to 9, pH of 6 to 9, or pH of 6.5 to 8.5). In some embodiments, suitable conditions include a temperature of 4° C. to 50° C. (e.g., 10° C. to 40° C., 15° C. to 40° C., 20° C. to 40° C., or 30° C. to 40° C.),

In some embodiments the linear polyribonucleotide is produced from a deoxyribonucleic acid, e.g., a deoxyribonucleic acid described herein, such as a DNA vector, a linearized DNA vector, or a cDNA. In some embodiments, the linear polyribonucleotide is transcribed from the deoxyribonucleic acid by transcription in a cell-free system (e.g., in vitro transcription).

### Methods of Production in a Cell

The disclosure also provides methods of producing a circular RNA in a cell, e.g., a prokaryotic cell or a eukaryotic cell. In some embodiments, an exogenous polyribonucleotide is provided to a cell (e.g., a linear polyribonucleotide described herein or a DNA molecule encoding for the transcription of a linear polyribonucleotide described here). The linear polyribonucleotides may be transcribed in the cell from an exogenous DNA molecule provided to the cell. The linear polyribonucleotide may be transcribed in the cell from an exogenous recombinant DNA molecule transiently provided to the cell. In some embodiments, the exogenous DNA molecule does not integrate into the cell's genome. In some embodiments, the linear polyribonucleotide is transcribed in the cell from a recombinant DNA molecule that is incorporated into the cell's genome.

In some embodiments, the cell is a prokaryotic cell. In some embodiments, the prokaryotic cell including the polyribonucleotides described herein may be a bacterial cell or an archaeal cell. For example, the prokaryotic cell including the polyribonucleotides described herein may be E. coli, halophilic archaea (e.g., Haloferax volcaniii), Sphingomonas, cyanobacteria (e.g., Synechococcus elongatus, Spirulina (Arthrospira) spp., and Synechocystis spp.), Streptomyces, actinomycetes (e.g., Nonomuraea, Kitasatospora, or Thermobifida), Bacillus spp. (e.g., Bacillus subtilis, Bacillus anthracis, Bacillus cereus), betaproteobacteria (e.g., Burkholderia), alphaproteobacterial (e.g., Agrobacterium), Pseudomonas (e.g., Pseudomonas putida), and enterobacteria. The prokaryotic cells may be grown in a culture medium. The prokaryotic cells may be contained in a bioreactor.

In some embodiments, the cell is a eukaryotic cell. In some embodiments, the eukaryotic cell including the polyribonucleotides described herein is a unicellular eukaryotic cell. In some embodiments, the unicellular eukaryotic is a unicellular fungal cell such as a yeast cell (e.g., Saccharomyces cerevisiae and other Saccharomyces spp., Brettanomyces spp., Schizosaccharomyces spp., Torulaspora spp., and Pichia spp.). In some embodiments, the unicellular eukaryotic cell is a unicellular animal cell. A unicellular animal cell may be a cell isolated from a multicellular animal and grown in culture, or the daughter cells thereof. In some embodiments, the unicellular animal cell may be dedifferentiated. In some embodiments, the unicellular eukaryotic cell is a unicellular plant cell. A unicellular plant cell may be a cell isolated from a multicellular plant and grown in culture, or the daughter cells thereof. In some embodiments, the unicellular plant cell may be dedifferentiated. In some embodiments, the unicellular plant cell is from a plant callus. In embodiments, the unicellular cell is a plant cell protoplast. In some embodiments, the unicellular eukaryotic cell is a unicellular eukaryotic algal cell, such as a unicellular green alga, a diatom, a euglenid, or a dinoflagellate. Non-limiting examples of unicellular eukaryotic algae of interest include Dunaliella salina, Chlorella vulgaris, Chlorella zofingiensis, Haematococcus pluvialis, Neochloris oleoabundans and other Neochloris spp., Protosiphon botryoides, Botryococcus braunii, Cryptococcus spp., Chlamydomonas reinhardtii and other Chlamydomonas spp. In some embodiments, the unicellular eukaryotic cell is a protist cell. In some embodiments, the unicellular eukaryotic cell is a protozoan cell.

In some embodiments, the eukaryotic cell is a cell of a multicellular eukaryote. For example, the multicellular eukaryote may be selected from the group consisting of a vertebrate animal, an invertebrate animal, a multicellular fungus, a multicellular alga, and a multicellular plant. In some embodiments, the eukaryotic organism is a human. In some embodiments, the eukaryotic organism is a non-human vertebrate animal. In some embodiments, the eukaryotic organism is an invertebrate animal. In some embodiments, the eukaryotic organism is a multicellular fungus. In some embodiments, the eukaryotic organism is a multicellular plant. In embodiments, the eukaryotic cell is a cell of a human or a cell of a non-human mammal such as a non-human primate (e.g., monkeys, apes), ungulate (e.g., bovids including cattle, buffalo, bison, sheep, goat, and musk ox; pig; camelids including camel, llama, and alpaca; deer, antelope; and equids including horse and donkey), carnivore (e.g., dog, cat), rodent (e.g., rat, mouse, guinea pig, hamster, squirrel), or lagomorph (e.g., rabbit, hare). In embodiments, the eukaryotic cell is a cell of a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the eukaryotic cell is a cell of an arthropod (e.g., insects, arachnids, crustaceans), a nematode, an annelid, a helminth, or a mollusc. In embodiments, the eukaryotic cell is a cell of a multicellular plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the eukaryotic cell is a cell of a eukaryotic multicellular alga.

The eukaryotic cells may be grown in a culture medium. The eukaryotic cells may be contained in a bioreactor.

### Methods of Purification

One or more purification steps may be included in the methods described herein. For example, in some embodiments, the linear polyribonucleotide is substantively enriched or pure (e.g., purified) prior to self-splicing the linear polyribonucleotide. In other embodiments, the linear polyribonucleotide is not purified prior to self-splicing the linear polyribonucleotide. In some embodiments, the resulting circular RNA is purified.

Purification may include separating or enriching the desired reaction product from one or more undesired components, such as any unreacted stating material, byproducts, enzymes, or other reaction components. For example, purification of linear polyribonucleotide following transcription in a cell-free system (e.g., in vitro transcription) may include separation or enrichment from the DNA template prior to self-splicing the linear polyribonucleotide. Purification of the circular RNA product following splicing may be used to separate or enrich the circular RNA from its corresponding linear RNA. Methods of purification of RNA are known to those of skill in the art and include enzymatic purification or by chromatography.

In some embodiments, the methods of purification result in a circular polyribonucleotide that has less than 50% (e.g., less than 40%, 30%, 20%, 10%, 5%, 4%, 3%, 2%, or 1%) linear polyribonucleotides.

### Bioreactors

In some embodiments, any method of producing a circular polyribonucleotide described herein may be performed in a bioreactor. A bioreactor refers to any vessel in which a chemical or biological process is carried out which involves organisms or biochemically active substances derived from such organisms. Bioreactors may be compatible with the cell-free methods for production of circular RNA described herein. A vessel for a bioreactor may include a culture flask, a dish, or a bag that may be single use (disposable), autoclavable, or sterilizable. A bioreactor may be made of glass, or it may be polymer-based, or it may be made of other materials.

Examples of bioreactors include, without limitation, stirred tank (e.g., well mixed) bioreactors and tubular (e.g., plug flow) bioreactors, airlift bioreactors, membrane stirred tanks, spin filter stirred tanks, vibromixers, fluidized bed reactors, and membrane bioreactors. The mode of operating the bioreactor may be a batch or continuous processes. A bioreactor is continuous when the reagent and product streams are continuously being fed and withdrawn from the system. A batch bioreactor may have a continuous recirculating flow, but no continuous feeding of reagents or product harvest.

Some methods of the present disclosure are directed to large-scale production of circular polyribonucleotides. For large-scale production methods, the method may be performed in a volume of 1 liter (L) to 50 L, or more (e.g., 5 L, 10 L, 15 L, 20 L, 25 L, 30 L, 35 L, 40 L, 45 L, 50 L, or more). In some embodiments, the method may be performed in a volume of 5 L to 10 L, 5 L to 15 L, 5 L to 20 L, 5 L to 25 L, 5 L to 30 L, 5 L to 35 L, 5 L to 40 L, 5 L to 45 L, 10 L to 15 L, 10 L to 20 L, 10 L to 25 L, 20 L to 30 L, 10 L to 35 L, 10 L to 40 L, 10 L to 45 L, 10 L to 50 L, 15 L to 20 L, 15 L to 25 L, 15 L to 30 L, 15 L to 35 L, 15 L to 40 L, 15 L to 45 L, or 15 to 50 L.

In some embodiments, a bioreactor may produce at least 1 g of circular RNA. In some embodiments, a bioreactor may produce 1-200 g of circular RNA (e.g., 1-10 g, 1-20 g, 1-50 g, 10-50 g, 10-100 g, 50-100 g, of 50-200 g of circular RNA). In some embodiments, the amount produced is measured per liter (e.g., 1-200 g per liter), per batch or reaction (e.g., 1-200 g per batch or reaction), or per unit time (e.g., 1-200 g per hour or per day).

In some embodiments, more than one bioreactor may be utilized in series to increase the production capacity (e.g., one, two, three, four, five, six, seven, eight, or nine bioreactors may be used in series).

### Methods of Use

In some embodiments, circular polyribonucleotides made as described herein are used as effectors in therapy or agriculture.

For example, a circular polyribonucleotide made by the methods described herein may be administered to a subject (e.g., in a pharmaceutical, veterinary, or agricultural composition). In some embodiments, the subject is a vertebrate animal (e.g., mammal, bird, fish, reptile, or amphibian). In some embodiments, the subject is a human. In some embodiments, the subject is a non-human mammal. In embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusk. In embodiments, the subject is an invertebrate agricultural pest or an invertebrate that is parasitic on an invertebrate or vertebrate host. In embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses.

In some embodiments, the disclosure provides a method of modifying a subject by providing to the subject a composition or formulation described herein. In some embodiments, the composition or formulation is or includes a nucleic acid molecule (e.g., a DNA molecule or an RNA molecule described herein), and the polynucleotide is provided to a eukaryotic subject. In some embodiments, the composition or formulation is or includes or a eukaryotic or prokaryotic cell including a nucleic acid described herein.

In some embodiments, the disclosure provides a method of treating a condition in a subject in need thereof by providing to the subject a composition or formulation described herein. In some embodiments, the composition or formulation is or includes a nucleic acid molecule (e.g., a DNA molecule or an RNA molecule described herein), and the polynucleotide is provided to a eukaryotic subject. In some embodiments, the composition or formulation is or includes a eukaryotic or prokaryotic cell including a nucleic acid described herein.

In some embodiments, the disclosure provides a method of providing a circular polyribonucleotide to a subject by providing a eukaryotic or prokaryotic cell include a polynucleotide described herein to the subject.

### Formulations

In some embodiments of the present disclosure a circular polyribonucleotide described herein may be formulated in composition, e.g., a composition for delivery to a cell, a plant, an invertebrate animal, a non-human vertebrate animal, or a human subject, e.g., an agricultural, veterinary, or pharmaceutical composition. In some embodiments, the circular polyribonucleotide is formulated in a pharmaceutical composition. In some embodiments, a composition includes a circular polyribonucleotide and a diluent, a carrier, an adjuvant, or a combination thereof. In a particular embodiment, a composition includes a circular polyribonucleotide described herein and a carrier or a diluent free of any carrier. In some embodiments, a composition including a circular polyribonucleotide with a diluent free of any carrier is used for naked delivery of the circular polyribonucleotide to a subject.

### Salts

In some cases, a composition or pharmaceutical composition provided herein comprises one or more salts. For controlling the tonicity, a physiological salt such as sodium salt can be included a composition provided herein. Other salts can comprise potassium chloride, potassium dihydrogen phosphate, disodium phosphate, and/or magnesium chloride, or the like. In some cases, the composition is formulated with one or more pharmaceutically acceptable salts. The one or more pharmaceutically acceptable salts can comprise those of the inorganic ions, such as, for example, sodium, potassium, calcium, magnesium ions, and the like. Such salts can comprise salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid, or maleic acid. The polyribonucleotide can be present in either linear or circular form.

### Buffers/pH

A composition or pharmaceutical composition provided herein can comprise one or more buffers, such as a Tris buffer; a borate buffer; a succinate buffer; a histidine buffer (e.g., with an aluminum hydroxide adjuvant); or a citrate buffer. Buffers, in some cases, are included in the 5-20 mM range.

A composition or pharmaceutical composition provided herein can have a pH between about 5.0 and about 8.5, between about 6.0 and about 8.0, between about 6.5 and about 7.5, or between about 7.0 and about 7.8. The composition or pharmaceutical composition can have a pH of about 7. The polyribonucleotide can be present in either linear or circular form.

### Detergents/Surfactants

A composition or pharmaceutical composition provided herein can comprise one or more detergents and/or surfactants, depending on the intended administration route, e.g., polyoxyethylene sorbitan esters surfactants (commonly referred to as “Tweens”), e.g., polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, e.g., octoxynol-9 (Triton X-100, or t-octylphenoxypolyethoxyethanol); (octylphenoxy) polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as “SPANs”), such as sorbitan trioleate (Span 85) and sorbitan monolaurate, an octoxynol (such as octoxynol-9 (Triton X-100) or t-octylphenoxypolyethoxyethanol), a cetyl trimethyl ammonium bromide (“CTAB”), or sodium deoxycholate. The one or more detergents and/or surfactants can be present only at trace amounts. In some cases, the composition can include less than 1 mg/ml of each of octoxynol-10 and polysorbate 80. Non-ionic surfactants can be used herein. Surfactants can be classified by their “HLB” (hydrophile/lipophile balance). In some cases, surfactants have a HLB of at least 10, at least 15, and/or at least 16. The polyribonucleotide can be present in either linear or circular form.

### Diluents

In some embodiments, a composition of the disclosure includes a circular polyribonucleotide and a diluent. In some embodiments, a composition of the disclosure includes a linear polyribonucleotide and a diluent.

A diluent can be a non-carrier excipient. A non-carrier excipient serves as a vehicle or medium for a composition, such as a circular polyribonucleotide as described herein. A non-carrier excipient serves as a vehicle or medium for a composition, such as a linear polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. A non-carrier excipient can be any inactive ingredient suitable for administration to a non-human animal, for example, suitable for veterinary use. Modification of compositions suitable for administration to humans in order to render the compositions suitable for administration to various animals is well understood, and the ordinarily skilled veterinary pharmacologist can design and/or perform such modification with merely ordinary, if any, experimentation.

In some embodiments, the circular polyribonucleotide may be delivered as a naked delivery formulation, such as including a diluent. A naked delivery formulation delivers a circular polyribonucleotide, to a cell without the aid of a carrier and without modification or partial or complete encapsulation of the circular polyribonucleotide, capped polyribonucleotide, or complex thereof.

A naked delivery formulation is a formulation that is free from a carrier and wherein the circular polyribonucleotide is without a covalent modification that binds a moiety that aids in delivery to a cell or without partial or complete encapsulation of the circular polyribonucleotide. In some embodiments, a circular polyribonucleotide without a covalent modification that binds a moiety that aids in delivery to a cell is a polyribonucleotide that is not covalently bound to a protein, small molecule, a particle, a polymer, or a biopolymer. A circular polyribonucleotide without covalent modification that binds a moiety that aids in delivery to a cell does not contain a modified phosphate group. For example, a circular polyribonucleotide without a covalent modification that binds a moiety that aids in delivery to a cell does not contain phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, a naked delivery formulation is free of any or all of: transfection reagents, cationic carriers, carbohydrate carriers, nanoparticle carriers, or protein carriers. In some embodiments, a naked delivery formulation is free from phtoglycogen octenyl succinate, phytoglycogen beta-dextrin, anhydride-modified phytoglycogen beta-dextrin, lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N-trimethylammonium chloride (DOTMA), I-[2-(oleoyloxy)ethyl]-2-oleyl-3-(2-hydroxyethyl) imidazolinium chloride (DOTIM), 2,3-dioleyloxy-N-[2 (sperminecarboxamido)ethyl]-N,N-dimethyl-I-propanaminium trifluoroacetate (DOSPA), 3B—[N—(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride (DODAC), human serum albumin (HSA), low-density lipoprotein (LDL), high-density lipoprotein (HDL), or globulin.

In certain embodiments, a naked delivery formulation includes a non-carrier excipient. In some embodiments, a non-carrier excipient includes an inactive ingredient that does not exhibit a cell-penetrating effect. In some embodiments, a non-carrier excipient includes a buffer, for example PBS. In some embodiments, a non-carrier excipient is a solvent, a non-aqueous solvent, a diluent, a suspension aid, a surface-active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation includes a diluent. A diluent may be a liquid diluent or a solid diluent. In some embodiments, a diluent is an RNA solubilizing agent, a buffer, or an isotonic agent. Examples of an RNA solubilizing agent include water, ethanol, methanol, acetone, formamide, and 2-propanol. Examples of a buffer include 2-(N-morpholino) ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[1,3-dihydroxy-2-(hydroxymethyl) propan-2-yl]amino]ethanesulfonic acid (TES), 3-(N-morpholino) propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

### Carriers

In some embodiments, a composition of the disclosure includes a circular polyribonucleotide and a carrier. In some embodiments, a composition of the disclosure includes a linear polyribonucleotide and a carrier.

In certain embodiments, a composition includes a circular polyribonucleotide as described herein in a vesicle or other membrane-based carrier. In certain embodiments, a composition includes a linear polyribonucleotide as described herein in a vesicle or other membrane-based carrier.

In other embodiments, a composition includes the circular polyribonucleotide in or via a cell, vesicle or other membrane-based carrier. In other embodiments, a composition includes the linear polyribonucleotide in or via a cell, vesicle or other membrane-based carrier. In one embodiment, a composition includes the circular polyribonucleotide in liposomes or other similar vesicles. In one embodiment, a composition includes the linear polyribonucleotide in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral, or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi: 10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi: 10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

In certain embodiments, a composition of the disclosure includes a circular polyribonucleotide and lipid nanoparticles, for example lipid nanoparticles described herein. In certain embodiments, a composition of the disclosure includes a linear polyribonucleotide and lipid nanoparticles. Lipid nanoparticles are another example of a carrier that provides a biocompatible and biodegradable delivery system for a circular polyribonucleotide molecule as described herein. Lipid nanoparticles are another example of a carrier that provides a biocompatible and biodegradable delivery system for a linear polyribonucleotide molecule as described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi: 10.3390/nano7060122.

Additional non-limiting examples of carriers include carbohydrate carriers (e.g., an anhydride-modified phytoglycogen or glycogen-type material), protein carriers (e.g., a protein covalently linked to the circular polyribonucleotide or a protein covalently linked to the linear polyribonucleotide), or cationic carriers (e.g., a cationic lipopolymer or transfection reagent). Non-limiting examples of carbohydrate carriers include phtoglycogen octenyl succinate, phytoglycogen beta-dextrin, and anhydride-modified phytoglycogen beta-dextrin. Non-limiting examples of cationic carriers include lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N-trimethylammonium chloride (DOTMA), I-[2-(oleoyloxy)ethyl]-2-oleyl-3-(2-hydroxyethyl) imidazolinium chloride (DOTIM), 2,3-dioleyloxy-N-[2 (sperminecarboxamido)ethyl]-N,N-dimethyl-I-propanaminium trifluoroacetate (DOSPA), 3B—[N—(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N, N-dimethyl-N-hydroxyethyl ammonium bromide (DMRIE), and N,N-dioleyl-N,N-dimethylammonium chloride (DODAC). Non-limiting examples of protein carriers include human serum albumin (HSA), low-density lipoprotein (LDL), high-density lipoprotein (HDL), or globulin.

Exosomes can also be used as drug delivery vehicles for a circular RNA composition or preparation described herein. Exosomes can be used as drug delivery vehicles for a linear polyribonucleotide composition or preparation described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a circular RNA composition or preparation described herein. Ex vivo differentiated red blood cells can also be used as a carrier for a linear polyribonucleotide composition or preparation described herein. See, e.g., International Patent Publication Nos. WO2015/073587; WO2017/123646; WO2017/123644; WO2018/102740; WO2016/183482; WO2015/153102; WO2018/151829; WO2018/009838; Shi et al. 2014. Proc Natl Acad Sci USA. 111 (28): 10131-10136; U.S. Pat. No. 9,644,180; Huang et al. 2017. Nature Communications 8: 423; Shi et al. 2014. Proc Natl Acad Sci USA. 111 (28): 10131-10136.

Fusosome compositions, e.g., as described in International Patent Publication No. WO2018/208728, can also be used as carriers to deliver a circular polyribonucleotide molecule described herein. Fusosome compositions, e.g., as described in WO2018/208728, can also be used as carriers to deliver a linear polyribonucleotide molecule described herein.

Virosomes and virus-like particles (VLPs) can also be used as carriers to deliver a circular polyribonucleotide molecule described herein to targeted cells. Virosomes and virus-like particles (VLPs) can also be used as carriers to deliver a linear polyribonucleotide molecule described herein to targeted cells.

Plant nanovesicles and plant messenger packs (PMPs), e.g., as described in International Patent Publication Nos. WO2011/097480, WO2013/070324, WO2017/004526, or WO2020/041784 can also be used as carriers to deliver the circular RNA composition or preparation described herein. Plant nanovesicles and plant messenger packs (PMPs) can also be used as carriers to deliver a linear polyribonucleotide composition or preparation described herein.

Microbubbles can also be used as carriers to deliver a circular polyribonucleotide molecule described herein. Microbubbles can also be used as carriers to deliver a linear polyribonucleotide molecule described herein. See, e.g., U.S. Pat. No. 7,115,583; Beeri, R. et al., Circulation. 2002 Oct. 1; 106 (14): 1756-1759; Bez, M. et al., Nat Protoc. 2019 April; 14 (4): 1015-1026; Hernot, S. et al., Adv Drug Deliv Rev. 2008 Jun. 30; 60 (10): 1153-1166; Rychak, J. J. et al., Adv Drug Deliv Rev. 2014 June; 72:82-93. In some embodiments, microbubbles are albumin-coated perfluorocarbon microbubbles.

The carrier including the circular polyribonucleotides described herein may include a plurality of particles. The particles may have median article size of 30 to 700 nanometers (e.g., 30 to 50, 50 to 100, 100 to 200, 200 to 300, 300 to 400, 400 to 500, 500 to 600, 600 to 700, 100 to 500, 50 to 500, or 200 to 700 nanometers). The size of the particle may be optimized to favor deposition of the payload, including the circular polyribonucleotide into a cell. Deposition of the circular polyribonucleotide into certain cell types may favor different particle sizes. For example, the particle size may be optimized for deposition of the circular polyribonucleotide into antigen presenting cells. The particle size may be optimized for deposition of the circular polyribonucleotide into dendritic cells. Additionally, the particle size may be optimized for depositions of the circular polyribonucleotide into draining lymph node cells.

### Lipid Nanoparticles

The compositions, methods, and delivery systems provided by the present disclosure may employ any suitable carrier or delivery modality described herein, including, in certain embodiments, lipid nanoparticles (LNPs). Lipid nanoparticles, in some embodiments, comprise one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol).

Lipids that can be used in nanoparticle formations (e.g., lipid nanoparticles) include, for example those described in Table 4 of WO2019217941, which is incorporated by reference—e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as I-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-I-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al. (2020), dx.doi.org/10.1021/acs.nanolett.0c01386, incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA (e.g., circular polyribonucleotide, linear polyribonucleotide)) described herein includes,

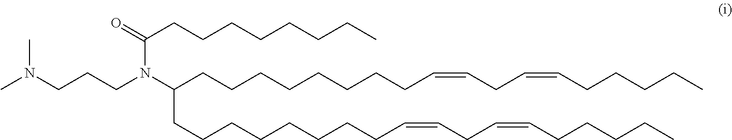 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver a polyribonucleotide (e.g., a circular polyribonucleotide, a linear polyribonucleotide) composition described herein to cells.

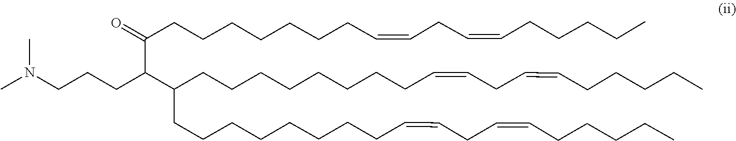 [SMILES: CCCCC/C=C\C/C=C\CCCCCCCCC(CCCCCCCC/C=C\C/C=C\CCCCC)C(CCCN(C)C)C(=O)CCCCCCC/C=C\C/C=C\CCCCC]

In some embodiments an LNP comprising Formula (ii) is used to deliver a polyribonucleotide (e.g., a circular polyribonucleotide, a linear polyribonucleotide) composition described herein to cells.

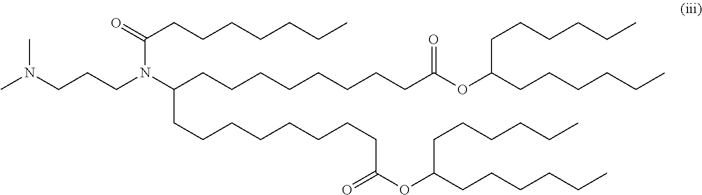 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver a polyribonucleotide (e.g., a circular polyribonucleotide, a linear polyribonucleotide) composition described herein to cells.

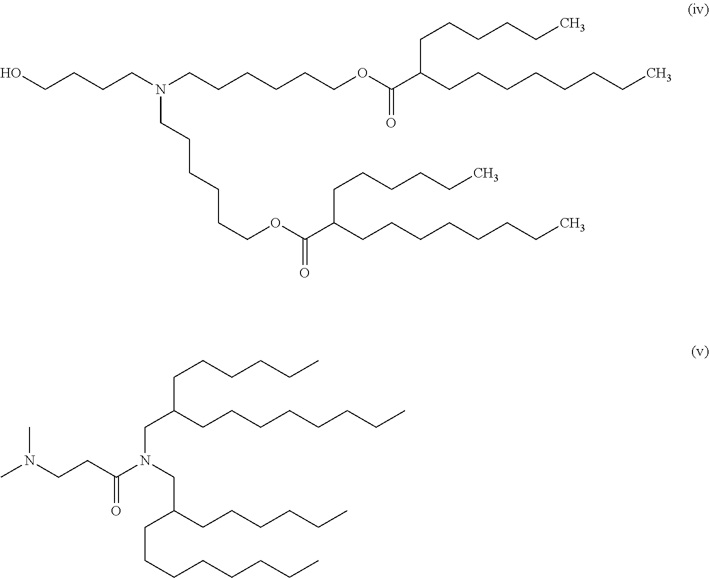

In some embodiments an LNP comprising Formula (v) is used to deliver a polyribonucleotide (e.g., a circular polyribonucleotide, a linear polyribonucleotide) composition described herein to cells.

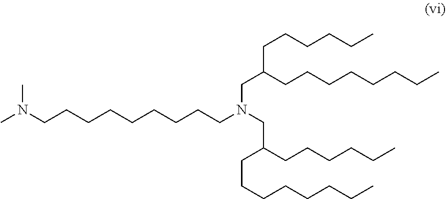 [SMILES: CCCCCCCCC(CCCCCC)CN(CCCCCCCCCN(C)C)CC(CCCCCC)CCCCCCCC]

In some embodiments an LNP comprising Formula (vi) is used to deliver a polyribonucleotide (e.g., a circular polyribonucleotide, a linear polyribonucleotide) composition described herein to cells.

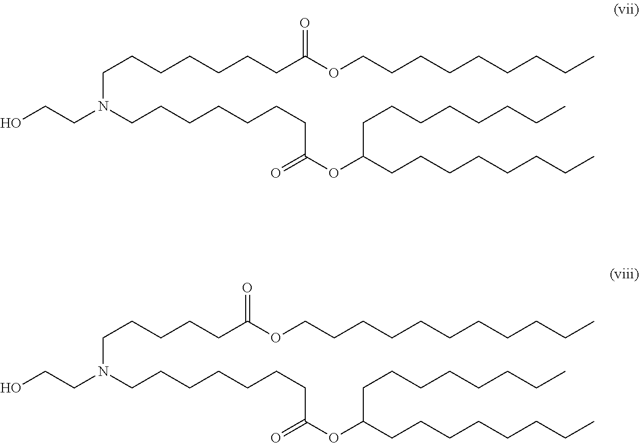 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP comprising Formula (viii) is used to deliver a polyribonucleotide (e.g., a circular polyribonucleotide, a linear polyribonucleotide) composition described herein to cells.

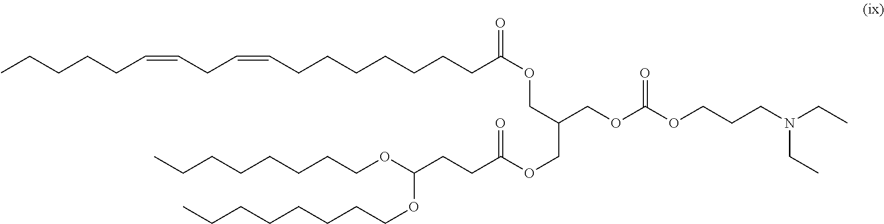 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP comprising Formula (ix) is used to deliver a polyribonucleotide (e.g., a circular polyribonucleotide, a linear polyribonucleotide) composition described herein to cells.

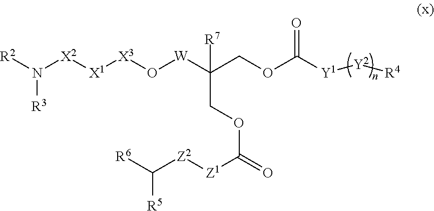

wherein
X¹ is O, NR¹, or a direct bond, X² is C2-5 alkylene, X³ is C(═O) or a direct bond, R¹ is H or Me, R³ is C1-3 alkyl, R² is C1-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

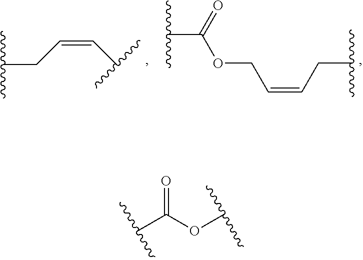 [SMILES: */C=C\C* ; *C/C=C\COC(*)=O ; *OC(*)=O]

(in either orientation), (in either orientation), (in either orientation),
n is 0 to 3, R⁴ is C1-15 alkyl, Z¹ is C1-6 alkylene or a direct bond,
Z² is

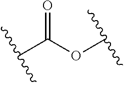 [SMILES: *OC(*)=O]

(in either orientation) or absent, provided that if Z¹ is a direct bond, Z² is absent;
R⁵ is C5-9 alkyl or C6-10 alkoxy, R⁶ is C5-9 alkyl or C6-10 alkoxy, W is methylene or a direct bond, and
R⁷ is H or Me, or a salt thereof, provided that if R³ and R² are C2 alkyls, X₁ is O, X² is linear C3 alkylene,
X³ is C(═O), Y¹ is linear Ce alkylene, (Y²)n-R⁴ is

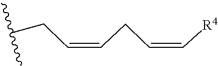

R⁴ is linear C5 alkyl, Z¹ is C2 alkylene, Z² is absent, W is methylene, and R⁷ is H, then R⁵ and R⁶ are not Cx alkoxy.

In some embodiments an LNP comprising Formula (xii) is used to deliver a polyribonucleotide (e.g., a circular polyribonucleotide, a linear polyribonucleotide) composition described herein to cells.

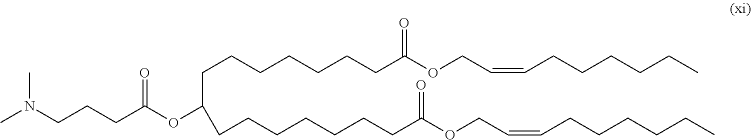 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver a polyribonucleotide (e.g., a circular polyribonucleotide, a linear polyribonucleotide) composition described herein to cells.

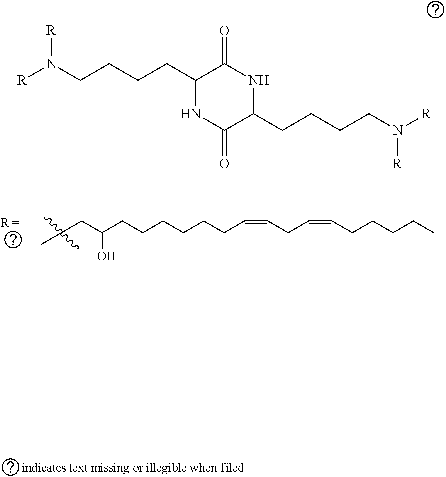

where R=

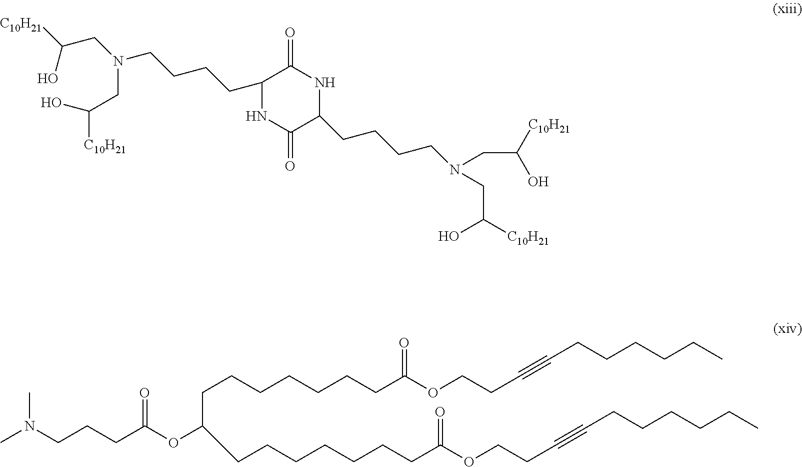

In some embodiments an LNP comprises a compound of Formula (xiii) and a compound of Formula (xiv).

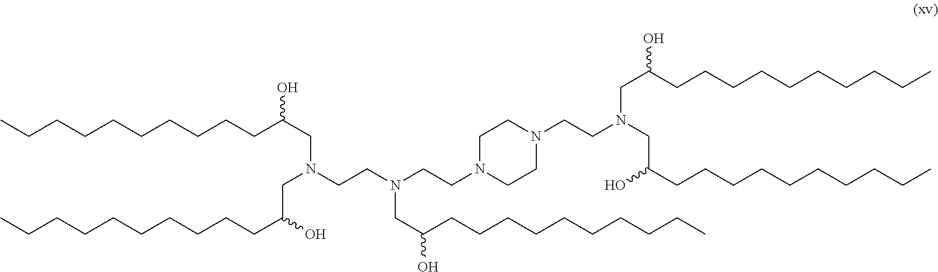 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP comprising Formula (xv) is used to deliver a polyribonucleotide (e.g., a circular polyribonucleotide, a linear polyribonucleotide) composition described herein to cells.

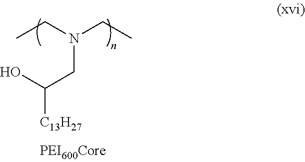

In some embodiments an LNP comprising a formulation of Formula (xvi) is used to deliver a polyribonucleotide (e.g., a circular polyribonucleotide, a linear polyribonucleotide) composition described herein to cells.

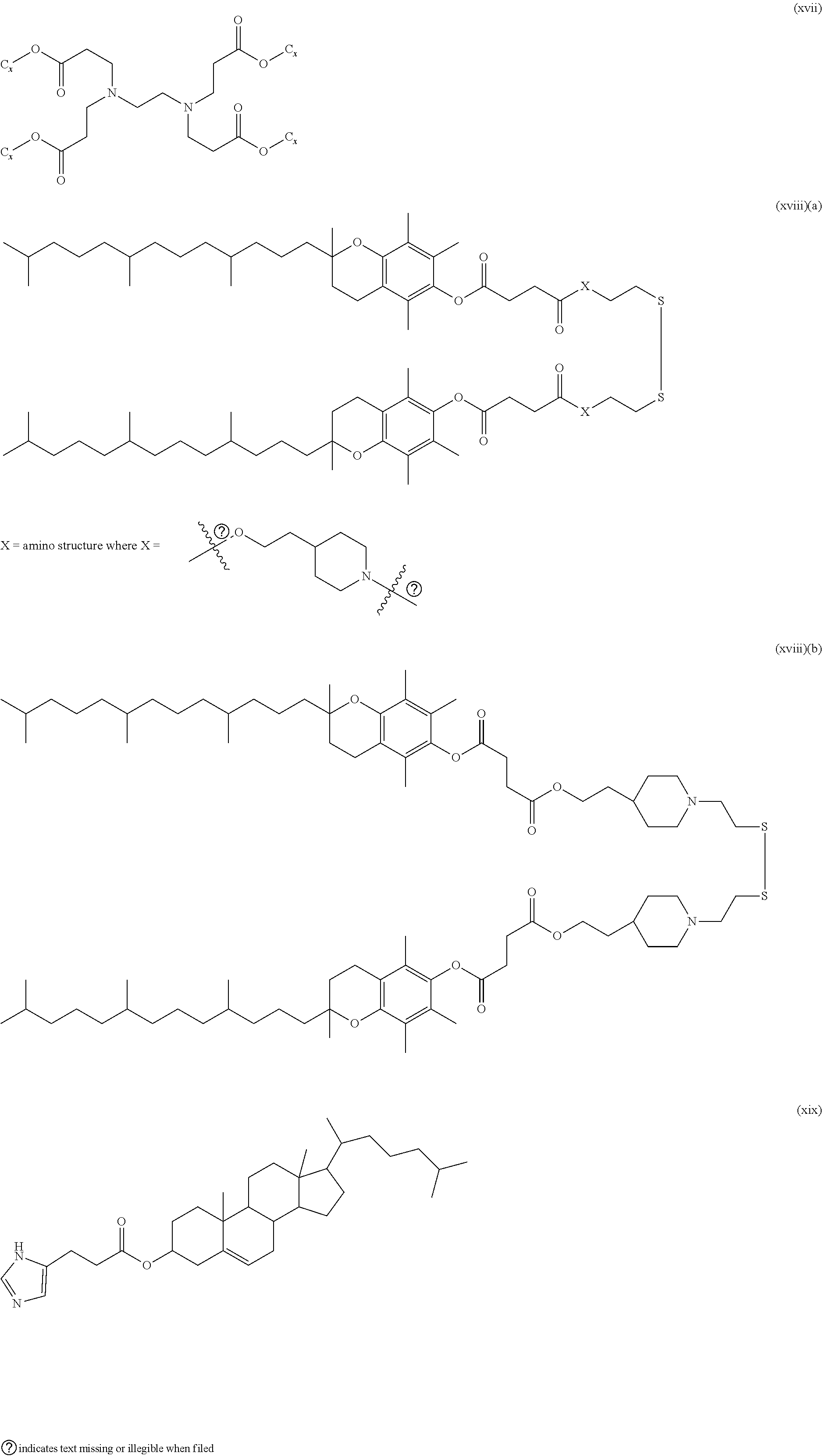

In some embodiments, a lipid compound used to form lipid nanoparticles for the delivery of compositions described herein, e.g., nucleic acid (e.g., RNA (e.g., circular polyribonucleotide, linear polyribonucleotide)) described herein is made by one of the following reactions:

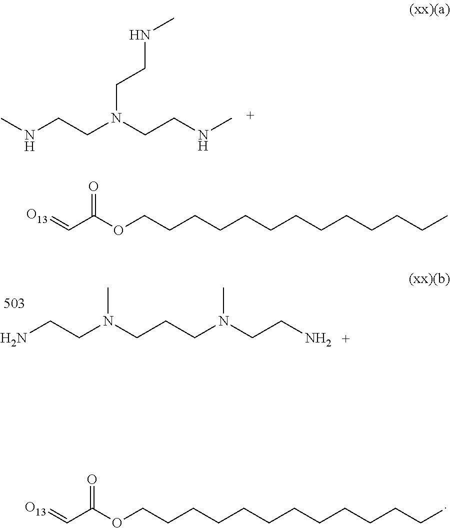

In some embodiments an LNP comprising Formula (xxi) is used to deliver a polyribonucleotide (e.g., a circular polyribonucleotide, a linear polyribonucleotide) composition described herein to cells. In some embodiments the LNP of Formula (xxi) is an LNP described by WO2021113777 (e.g., a lipid of Formula (1) such as a lipid of Table 1 of WO2021113777).

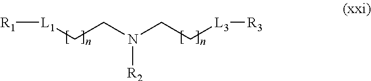

wherein
each n is independently an integer from 2-15; L₁ and L₃ are each independently —OC(O)—* or —C(O)O—*, wherein “*” indicates the attachment point to R₁ or R₃;

R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl) (alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkyl sulfonyl, and alkyl sulfonealkyl; and

R₂ is selected from a group consisting of:

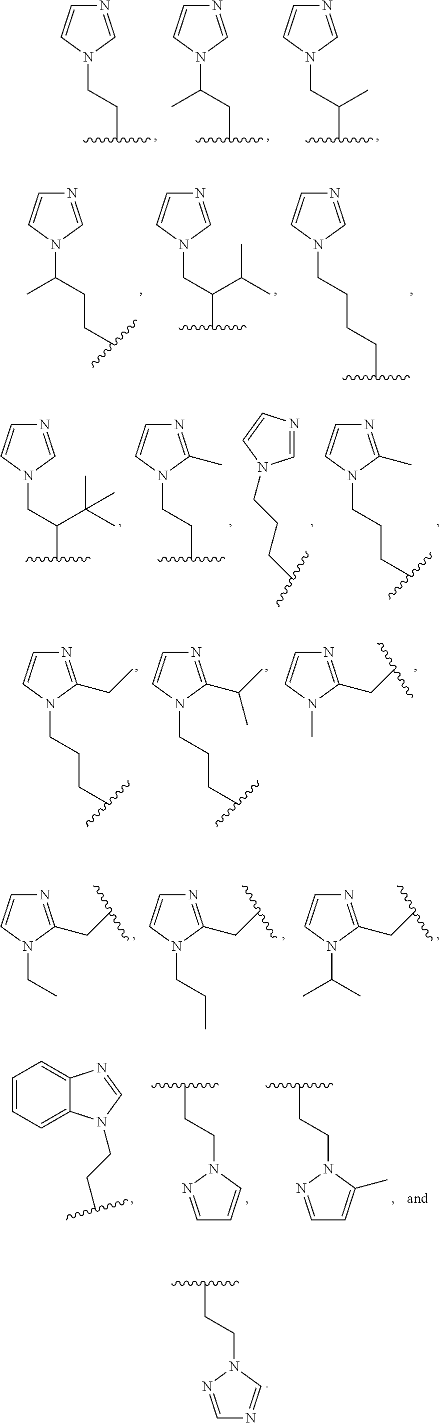

In some embodiments an LNP comprising Formula (xxii) is used to deliver a polyribonucleotide (e.g., a circular polyribonucleotide, a linear polyribonucleotide) composition described herein to cells. In some embodiments the LNP of Formula (xxii) is an LNP described by WO2021113777 (e.g., a lipid of Formula (2) such as a lipid of Table 2 of WO2021113777).

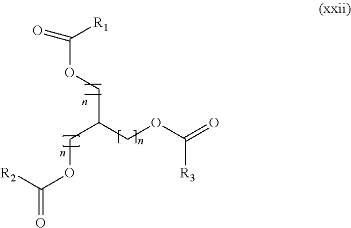

wherein
each n is independently an integer from 1-15;
R₁ and R₂ are each independently selected from a group consisting of:

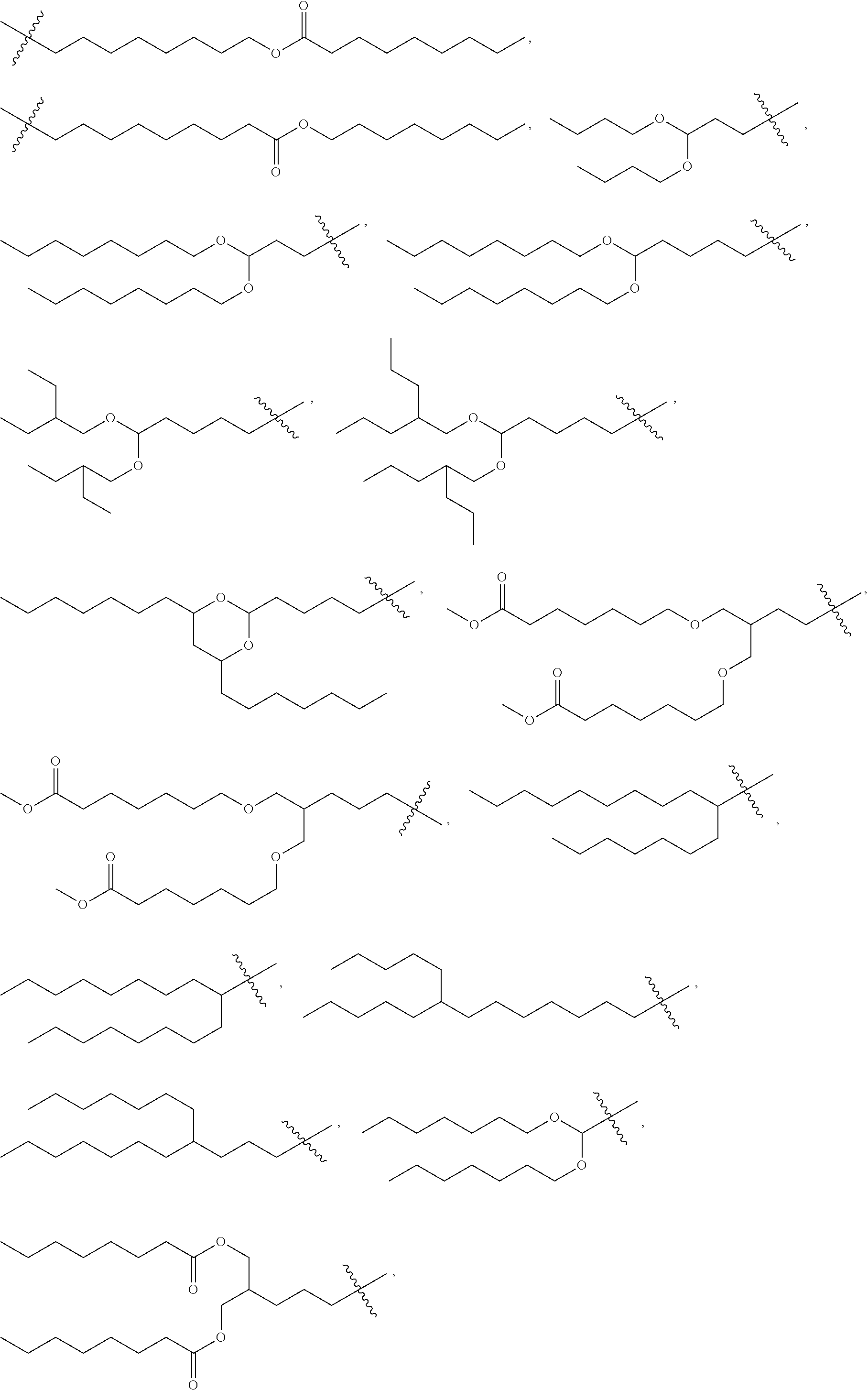

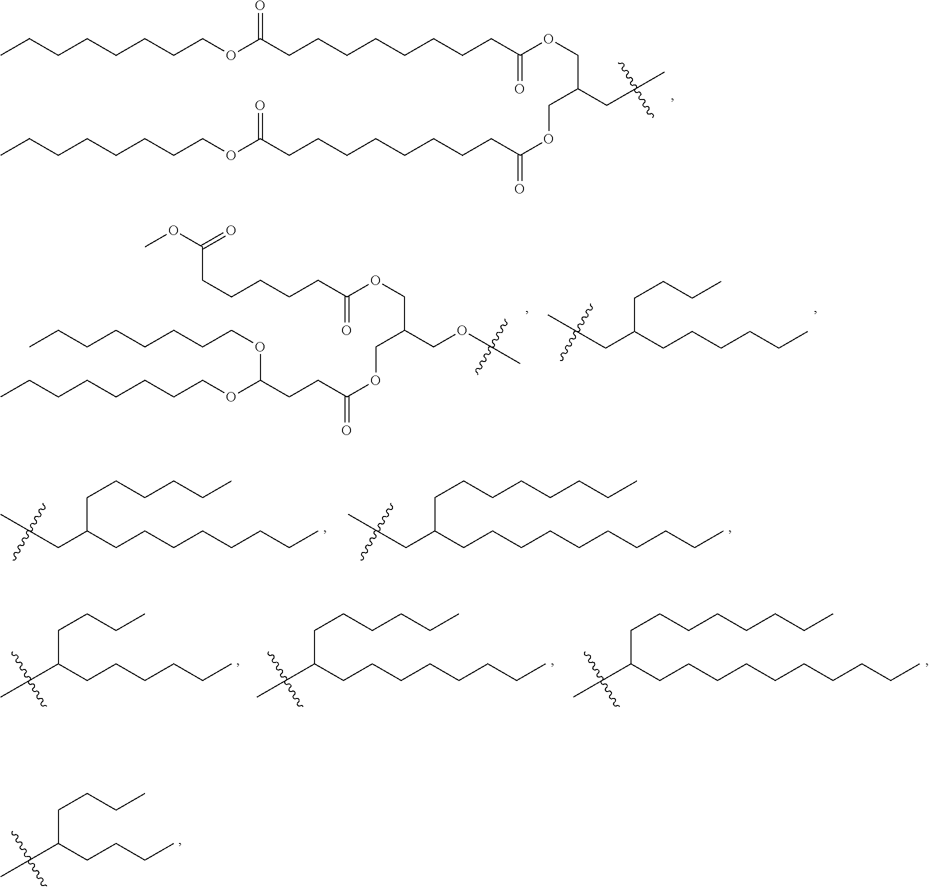

R₃ is selected from a group consisting of:

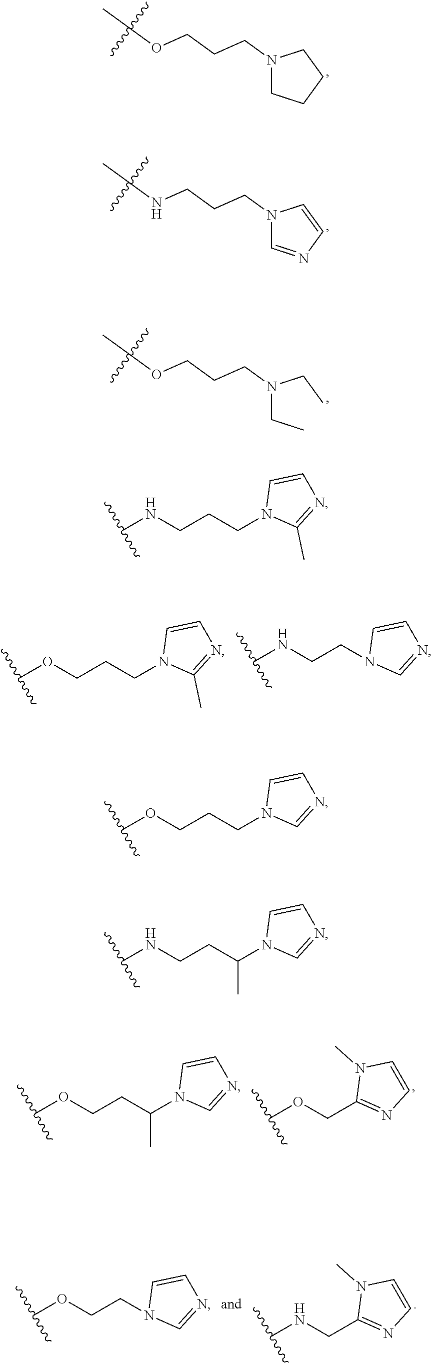

In some embodiments an LNP comprising Formula (xxiii) is used to deliver a polyribonucleotide (e.g., a circular polyribonucleotide, a linear polyribonucleotide) composition described herein to cells. In some embodiments the LNP of Formula (xxiii) is an LNP described by WO2021113777 (e.g., a lipid of Formula (3) such as a lipid of Table 3 of WO2021113777).

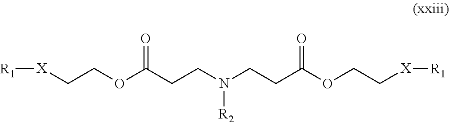

wherein
X is selected from —O—, —S—, or —OC(O)—*, wherein * indicates the attachment point to R₁;
R₁ is selected from a group consisting of:

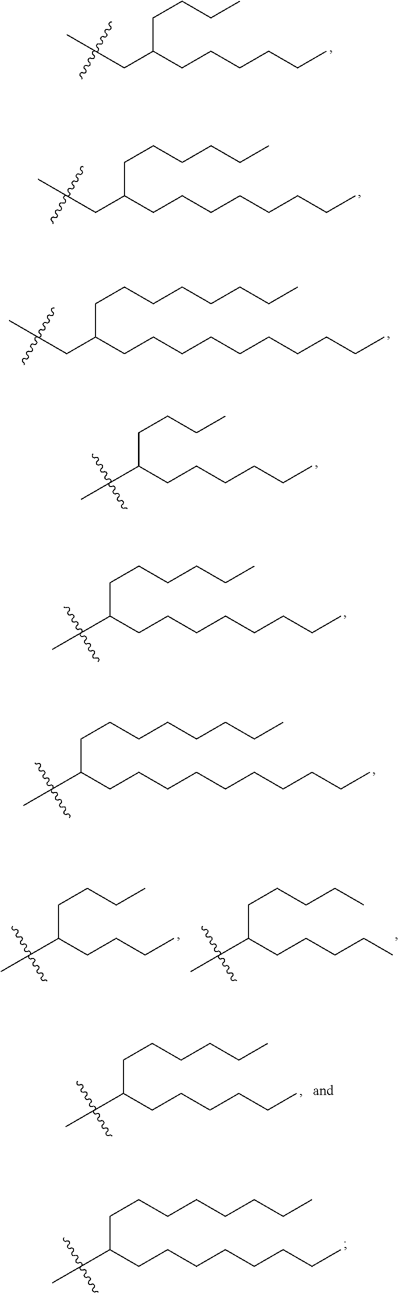

and R² is selected from a group consisting of:

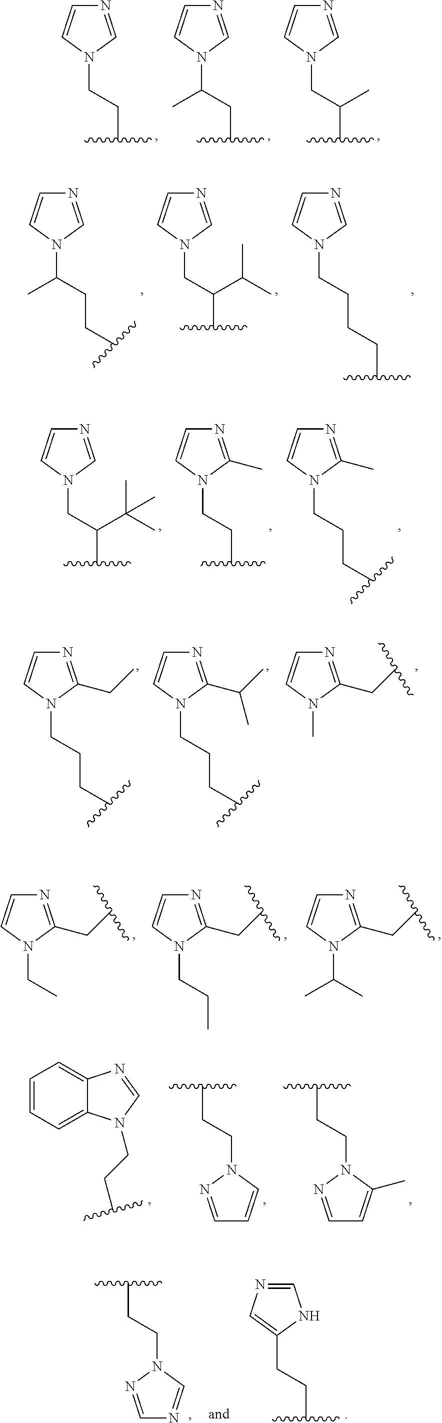

In some embodiments, a composition described herein (e.g., a nucleic acid (e.g., a circular polyribonucleotide, a linear polyribonucleotide) or a protein) is provided in an LNP that comprises an ionizable lipid. In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy)heptadecanedioate (L319), e.g., as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl)amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S,10R,13R,17R)-10,13-dimethyl-17-((R)-6-methylheptan-2-yl)-2,3,4,7,8,9,10,11,12,13,14,15,16,17-tetradecahydro-IH-cyclopenta[a]phenanthren-3-yl 3-(1H-imidazol-4-yl) propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol, and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a nucleic acid (e.g., RNA (e.g., a circular polyribonucleotide, a linear polyribonucleotide)) described herein, encapsulated within or associated with the lipid nanoparticle. In some embodiments, the nucleic acid is co-formulated with the cationic lipid. The nucleic acid may be adsorbed to the surface of an LNP, e.g., an LNP comprising a cationic lipid. In some embodiments, the nucleic acid may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or Ill of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or Ill of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; 1-5 or 1-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-O13 or 503-O13 of Whitehead et al; TS-P4C2 of U.S. Pat. No. 9,708,628; I of WO2020/106946; I of WO2020/106946; and (1), (2), (3), or (4) of WO2021/113777. Exemplary lipids further include a lipid of any one of Tables 1-16 of WO2021/113777.

In some embodiments, the ionizable lipid is MC3 (6Z,9Z,28Z,3IZ)-heptatriaconta-6,9,28,3l-tetraen-19-yl-4-(dimethylamino)butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-I-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-I-trans PE, I-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-cholestanol, 53-coprostanol, cholesteryl-(2-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as I-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-I-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, US2018/0028664, and WO2017/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (I-[8′-(Cholest-5-en-3 [beta]-oxy) carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid comprises PEG-DMG, 1,2-dimyristoyl-sn-glycero-3-phosphoethanolamine-N-[methoxy(polyethylene glycol)-2000]. In some embodiments, the PEG-lipid comprises a structure selected from:

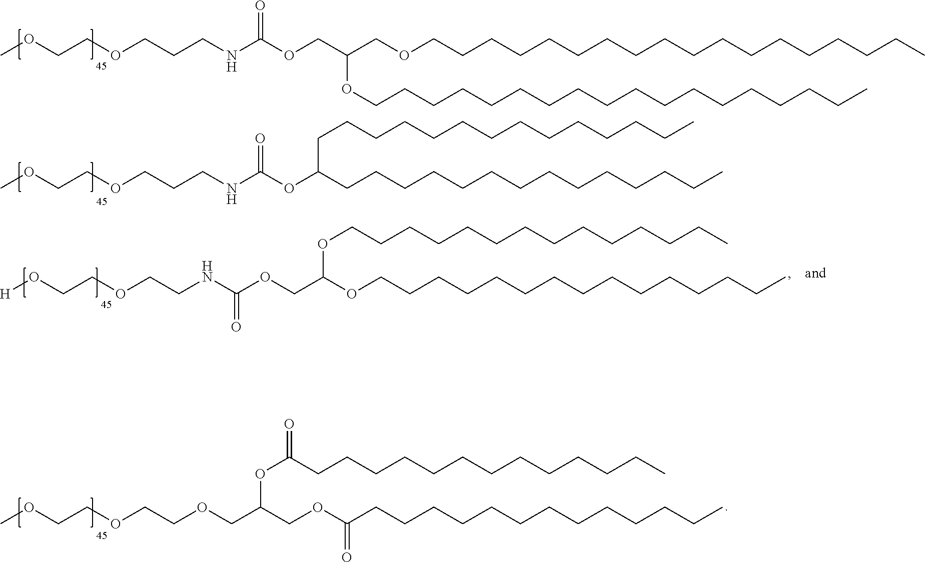

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g., phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of an LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

An LNP may optionally comprise one or more coatings. In some embodiments, a LNP may be formulated in a capsule, film, or table having a coating. A capsule, film, or tablet including a composition described herein may have any useful size, tensile strength, hardness or density.

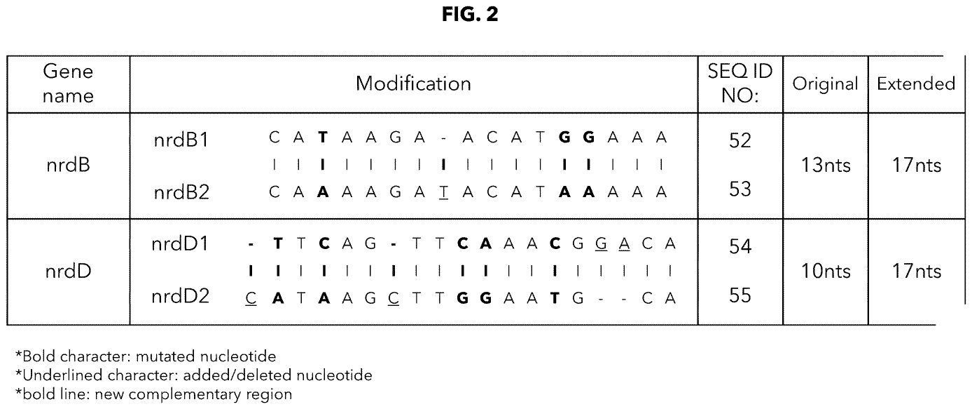

Additional exemplary lipids, formulations, methods, and characterization of LNPs are taught by WO2020/061457 and WO2021/113777, each of which is incorporated herein by reference in its entirety. Further exemplary lipids, formulations, methods, and characterization of LNPs are taught by Hou et al. Lipid nanoparticles for mRNA delivery. Nat Rev Mater (2021). doi.org/10.1038/s41578-021-00358-0, which is incorporated herein by reference in its entirety (see, for example, exemplary lipids and lipid derivatives of FIG. 2 of Hou et al.).

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51 (34): 8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference, and are useful for delivery of circular polyribonucleotides and linear polyribonucleotides described herein.

Additional specific LNP formulations useful for delivery of nucleic acids (e.g., circular polyribonucleotides, linear polyribonucleotides) are described in U.S. Pat. Nos. 8,158,601 and 8,168,775, both incorporated by reference, which include formulations used in patisiran, sold under the name ONPATTRO.

In embodiments, a polyribonucleotide (e.g., a circular polyribonucleotide, a linear polyribonucleotide) encoding at least a portion (e.g., an antigenic portion) of a protein or polypeptide described herein is formulated in an LNP, wherein: (a) the LNPs comprise a cationic lipid, a neutral lipid, a cholesterol, and a PEG lipid, (b) the LNPs have a mean particle size of between 80 nm and 160 nm, and (c) the polyribonucleotide. In embodiments, the polyribonucelotide (e.g., circular polyribonucleotide, linear polyribonucleotide) formulated in an LNP is a vaccine.

Exemplary dosing of polyribonucleotide (e.g., a circular polyribonucleotide, a linear polyribonucleotide) LNP may include about 0.1, 0.25, 0.3, 0.5, 1, 2, 3, 4, 5, 6, 8, 10, or 100 mg/kg (RNA). In some embodiments, a dose of a polyribonucleotide (e.g., a circular polyribonucleotide, a linear polyribonucleotide) antigenic composition described herein is between 30-200 mcg, e.g., 30 mcg, 50 mcg, 75 mcg, 100 mcg, 150 mcg, or 200 mcg.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their invention.

### Example 1: Design of T4 Phage nrdB or nrdD Self-Splicing Permuted Intron-Exon with Extended Annealing Region

This example describes the design of T4 phage nrdB or nrdD self-splicing permuted intron-exon (PIE) with extended annealing region.

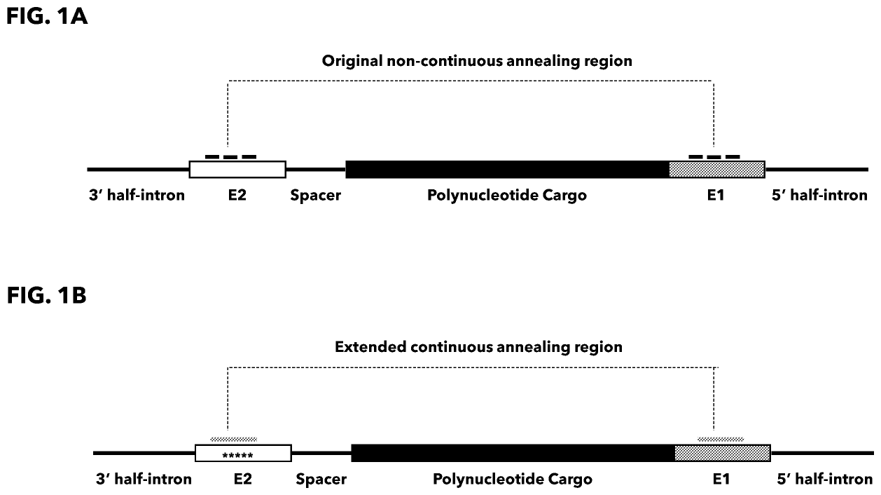

Schematics depicting exemplary designs of DNA constructs are provided in FIG. 1A and FIG. 1B. The constructs include, from 5′-to-3′: a 3′ half of group I catalytic intron fragment (T4 phage nrdB or nrdD 3′ half-intron), a 3′ splice site, a 3′ exon fragment (T4 phage nrdB or nrdD E2), a spacer element, a polynucleotide cargo, a 5′ exon fragment (T4 phage nrdB or nrdD E1), a 5′ splice site, and a 5′ half of group I catalytic intron fragment (T4 phage nrdB or nrdD 5′ half-intron).

The 3′ half intron fragment, 5′ half intron fragment, 3′ exon fragment, and 5′ exon fragment sequences for nrdB1, nrdB2, nrdD1, and nrdD2 used in the following experiments are shown below. Exon fragment sequences are shown in bold.

nrdB1: 3′ half-intron-3′ exon fragment
(SEQ ID NO: 1)
5′-TTGCAAAACAAGGTTCAACGACTAGTCTTCGGACGTAGGGTCAAGCGACTCGAAATGGGGAGAATCC
CTCCGGGATTGTGATATAGTCTGGACTGCATGGTAACATGCAGCAGTTCATAAGAGAACGGGTTGAG
AATTAGCGAGCTCAATCGAACATACGGTACCTTTAACTTCCATAAGAACATGGAAATCATGGAAGG
TAATGCCAAG-3′
nrdB1: 3′ half-intron
(SEQ ID NO: 2)
5′-TTGCAAAACAAGGTTCAACGACTAGTCTTCGGACGTAGGGTCAAGCGACTCGAAATGGGGAG
AATCCCTCCGGGATTGTGATATAGTCTGGACTGCATGGTAACATGCAGCAGTTCATAAGAGA
ACGGGTTGAGAATTAGCGAGCTCAATCGAACATACG-3′
nrdB1: 3′exon fragment
(SEQ ID NO: 3)
5′-GTACCTTTAACTTCCATAAGAACATGGAAATCATGGAAGGTAATGCCAAG-3′
nrdB1: 5′ exon fragment-5′ half-intron
(SEQ ID NO: 4)
5′-TTTTTATGTATCTTTTGCGTAAAATGCGCCTTTAAACGGTAACGTTTATCGAAAACTCCTTTAATTGCT
GGAAAGTCCTTTATGGAAAACTAGCAGCCAAGGTTTTGCTT-3′
nrdB1: 5′ exon fragment
(SEQ ID NO: 5)
5′-TTTTTATGTATCTTTTGCGT-3′
nrdB1: 5′ half-intron
(SEQ ID NO: 6)
5′-AAAATGCGCCTTTAAACGGTAACGTTTATCGAAAACTCCTTTAATTGCTGGAAAGTCCTTTAT
G GAAAACTAGCAGCCAAGGTTTTGCTT-3′
nrdB2: 3′ half-intron-3′ exon fragment
(SEQ ID NO: 7)
5′-TTGCAAAACAAGGTTCAACGACTAGTCTTCGGACGTAGGGTCAAGCGACTCGAAATGGGGAGAATCC
CTCCGGGATTGTGATATAGTCTGGACTGCATGGTAACATGCAGCAGTTCATAAGAGAACGGGTTGAG
AATTAGCGAGCTCAATCGAACATACGGTACCTTTAACTTCCAAAAGATACATAAAAATCATGGAAG
GTAATGCCAAG-3′
nrdB2: 3′ half-intron
(SEQ ID NO: 2)
5′-TTGCAAAACAAGGTTCAACGACTAGTCTTCGGACGTAGGGTCAAGCGACTCGAAATGGGGAG
AATCCCTCCGGGATTGTGATATAGTCTGGACTGCATGGTAACATGCAGCAGTTCATAAGAGA
ACGGGTTGAGAATTAGCGAGCTCAATCGAACATACG-3′
nrdB2: 3′ exon fragment
(SEQ ID NO: 8)
5′-GTACCTTTAACTTCCAAAAGATACATAAAAATCATGGAAGGTAATGCCAAG-3′
nrdB2: 5′ exon fragment-5′ half-intron
(SEQ ID NO: 4)
5′-TTTTTATGTATCTTTTGCGTAAAATGCGCCTTTAAACGGTAACGTTTATCGAAAACTCCTTTAATTGCT
GGAAAGTCCTTTATGGAAAACTAGCAGCCAAGGTTTTGCTT-3′
nrdB2: 5′ exon fragment
(SEQ ID NO: 5)
5′-TTTTTATGTATCTTTTGCGT-3′
nrdB2: 5′ half-intron
(SEQ ID NO: 6)
5′-AAAATGCGCCTTTAAACGGTAACGTTTATCGAAAACTCCTTTAATTGCTGGAAAGTCCTTTAT
G GAAAACTAGCAGCCAAGGTTTTGCTT-3′
nrdD1: 3′ half-intron-3′ exon fragment
(SEQ ID NO: 9)
5′-CAGTAGCTGTAAATGCCCAACGACTATCCCTGATGAATGTAAGGGAGTAGGGTCAAGCGACCCGAAA
CGGCAGACAACTCTAAGAGTTGAAGATATAGTCTGAACTGCATGGTGACATGCAGCTGTTTATCCTC
GTATAAATATGAATACGAGGTGAAACGATGAAATGAATTACATTGTTTCATATAAACGGGTAGAGAAG
TAGCGAACTCTACTGAACACATTGATGAAGTGAACACGTTATTCAGTTCAAACGGACAGACTCCTTT
TGTAACA-3′
nrdD1: 3′ half-intron
(SEQ ID NO: 10)
5′-CAGTAGCTGTAAATGCCCAACGACTATCCCTGATGAATGTAAGGGAGTAGGGTCAAGCGACC
CGAAACGGCAGACAACTCTAAGAGTTGAAGATATAGTCTGAACTGCATGGTGACATGCAGCT
GTTTATCCTCGTATAAATATGAATACGAGGTGAAACGATGAAATGAATTACATTGTTTCATATA
AACGGGTAGAGAAGTAGCGAACTCTACTGAACACATTG-3′
nrdD1: 3′ exon fragment
(SEQ ID NO: 11)
5′-ATGAAGTGAACACGTTATTCAGTTCAAACGGACAGACTCCTTTTGTAACA-3′
nrdD1: 5′ exon fragment-5′ half-intron
(SEQ ID NO: 12)
5′-TGCATTCCAAGCTTATGAGTTAACGTAAGTCAAGCTCATGTAAAATCTGCCTAAAACGGGAAACTCT
CACTGAGACAATCCGTTGCTAAATCAG-3′
nrdD1: 5′ exon fragment
(SEQ ID NO: 13)
5′-TGCATTCCAAGCTTATGAGT-3′
nrdD1: 5′ half-intron
(SEQ ID NO: 14)
5′-TAACGTAAGTCAAGCTCATGTAAAATCTGCCTAAAACGGGAAACTCTCACTGAGACAATCCG
TTGCTAAATCAG-3′
nrdD2: 3′ half-intron-3′ exon fragment
(SEQ ID NO: 15)
5′-CAGTAGCTGTAAATGCCCAACGACTATCCCTGATGAATGTAAGGGAGTAGGGTCAAGCGACCCGAA
ACGGCAGACAACTCTAAGAGTTGAAGATATAGTCTGAACTGCATGGTGACATGCAGCTGTTTATCCT
CGTATAAATATGAATACGAGGTGAAACGATGAAATGAATTACATTGTTTCATATAAACGGGTAGAGAA
GTAGCGAACTCTACTGAACACATTGATGAAGTGAACACGTTACATAAGCTTGGAATGCAGACTCCT
TTTGTAACA-3′
nrdD2: 3′ half-intron
(SEQ ID NO: 10)
5′-CAGTAGCTGTAAATGCCCAACGACTATCCCTGATGAATGTAAGGGAGTAGGGTCAAGCGACC
CGAAACGGCAGACAACTCTAAGAGTTGAAGATATAGTCTGAACTGCATGGTGACATGCAGCT
GTTTATCCTCGTATAAATATGAATACGAGGTGAAACGATGAAATGAATTACATTGTTTCATATA
AACGGGTAGAGAAGTAGCGAACTCTACTGAACACATTG-3′
nrdD2: 3′ exon fragment
(SEQ ID NO: 16)
5′-ATGAAGTGAACACGTTACATAAGCTTGGAATGCAGACTCCTTTTGTAACA-3′
nrdD2: 5′ exon fragment-5′ half-intron
(SEQ ID NO: 12)
5′-TGCATTCCAAGCTTATGAGTTAACGTAAGTCAAGCTCATGTAAAATCTGCCTAAAACGGGAAACTCT
CACTGAGACAATCCGTTGCTAAATCAG-3′
nrdD2: 5′ exon fragment
(SEQ ID NO: 13)
5′-TGCATTCCAAGCTTATGAGT-3′
nrdD2: 5′ half-intron
(SEQ ID NO: 14)
5′-TAACGTAAGTCAAGCTCATGTAAAATCTGCCTAAAACGGGAAACTCTCACTGAGACAATCCG
TTGCTAAATCAG-3′

nrdB E1-E2 has 13 nucleotides (nts) non-continuous complementary sequences and nrdD E1-E2 has 10 nts non-continuous complementary sequences (FIG. 1A, dashes on the E1 and E2; FIG. 2). To generate a construct that has an extended annealing region between E2 and E1, sequences in E2 were modified to have an extended annealing region with E1 (FIG. 1B, solid line on E1 and E2; FIG. 2). The total annealing region from group I permuted intron-exon (PIE) with an extended annealing region is 17 nucleotides.

Constructs that have the PIE with an original annealing region (nrdB1 or nrdD1) and annealing sequences with an extended annealing region (nrd2 or nrdD2) were designed to compare circularization efficiency. In this example, the constructs were designed to include a 50 nts spacer element, and a combination of an EMCV internal ribosome entry site (IRES) and an ORF as the polynucleotide cargo. SARS-COV-2 spike protein ORF (3822nts) was used as a model construct. The size of the circular RNA with the SARS-COV-2 spike protein ORF is 4.5 Kb.

Unmodified linear RNA was synthesized by in vitro transcription using T7 RNA polymerase from a DNA template in the presence of 12.5 mM of NTP. Template DNA was removed by treating with DNase for 20 minutes. Synthesized linear RNA was purified with an RNA clean up kit (New England Biolabs, T2050). Self-splicing occurred during transcription; no additional reaction was required. To monitor self-splicing efficiency, column purified, in vitro transcribed RNA was separated on an anionic exchange (AEX) column through HPLC. The percentage of linear and circular peaks were taken, and circularization efficiency was normalized with that of original constructs.

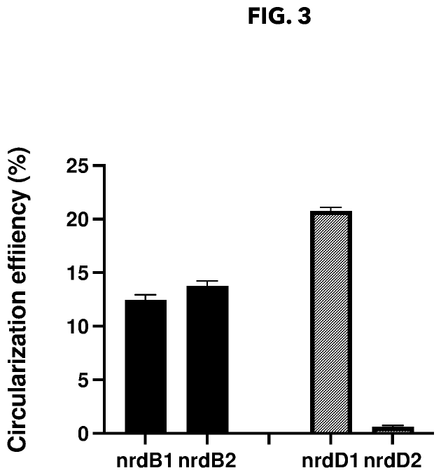

Extending the annealing sequence from 13 nts to 17 nts of the T4 phage nrdB self-splicing PIE (nrdB2) showed similar circularization efficiency with the nrdB1 PIE (nrdB1) (FIG. 3). This data indicate that circularization was not disrupted by extension of the annealing sequence in T4 phage nrdB self-splicing PIE.

Extending the annealing region of the T4 phage nrdD self-splicing PIE from 10 nts to 17 nts (nrdD2) significantly reduced circularization efficiency when compared with the nrdD PIE (nrdD1) (FIG. 3).

### Example 2: Protein Expression from Circular RNA Generated by T4 Phage Self-Splicing nrdB or nrdD PIE with an Extended Annealing Region

This example describes expression of circular RNA generated by T4 phage nrdB or nrdD self-splicing PIE with an extended annealing region.

DNA constructs with T4 phage PIE with original annealing sequences (nrdB1 or nrdD1) and extended annealing sequences (nrdB2 or nrdD2) are designed as described in Example 1. The constructs are designed to include a spacer element, and a combination of an EMCV IRES and SARS-CoV-2 spike protein ORF (3822nts) as the polynucleotide cargo. Linear RNA is synthesized by in vitro transcription using T7 RNA polymerase in the presence of 7.5 mM of NTP. Template DNA is removed by treating with DNase. Synthesized linear RNA is purified with an RNA clean up kit (New England Biolabs, T2050).

To compare expression of circular RNA encoding SARS-COV-2 spike protein, circular RNA generated by T4 phage nrdB or nrdD PIE with an original annealing region (nrdB1 or nrdD1) or an extended annealing region (nrdB2 or NrD2) are prepared. Hela cells (1.2 million cells per well in a 6 well plate) are transfected with 4 μmol of purified circular RNA using LIPOFECTAMINE® MessengerMAX (Invitrogen) transfection agent according to manufacturer's instructions. After 48-hour transfection, cells are harvested by trypsinization and resuspended in cold serum-free media. Cells are then stained with anti-SARS-COV-2 RBD antibody for one hour and subsequently incubated with anti-mouse IgG1 antibody AF647 for 30 min. The stained population is measured by flow cytometry.

### Example 3: Circular RNA Generated by T4 Phage Self-Splicing nrdB or nrdD PIE with an Extended Annealing Region

This example describes a circular RNA construct generated by T4 phage nrdB or nrdD self-splicing PIE with an extended annealing region.

DNA constructs with T4 phage PIE with original annealing sequences (nrdB1 or nrdD1) and extended annealing sequences (nrdB2 or nrdD2) were designed as described in Example 1. The constructs were designed to include a spacer element, and a combination of a modified CVB3 IRES and CFTR ORF (4443 nts) as the polynucleotide cargo. The size of the circular RNA was 5.5 Kb.

Unmodified linear RNA was synthesized by in vitro transcription using T7 RNA polymerase in the presence of 7.5 mM of NTP. Template DNA was removed by treating with DNase. Synthesized linear RNA was purified with an RNA clean up kit (New England Biolabs, T2050). Self-splicing occurred during transcription; no additional reaction was required.

### Example 4: Circularization Efficiency with T4 Phage nrdB or nrdD Self-Splicing Permuted Intron-Exon (PIE) with Extended Annealing Region

This example describes a circular RNA construct generated by T4 phage nrdB or nrdD self-splicing PIE with an extended annealing region.

Constructs that have the PIE with an original annealing region (nrdB1 or nrdD1) and annealing sequences with an extended annealing region (nrdB2 or nrdD2) were designed to compare circularization efficiency. In this example, the constructs were designed to include a different length 5′ or 3′ spacer element (0, 50 nt, or 120 nt), an EMCV or modified CVB3 internal ribosome entry site (IRES), and an ORF as the polynucleotide cargo. Human erythropoietin (hEPO) ORF was used as a model construct. The following constructs were tested.

TABLE 10
Constructs tested for circularization

| Construct | 5′ spacer | IRES | ORF | 3′ spacer |
|---|---|---|---|---|
| NrdB1 | AT50 | WTEMCV | hEPO | None |
| NrdB2 | AT50 | modified CVB3 | hEPO | AU50 |
| NrdD1 | AT50 | WTEMCV | hEPO | None |
| NrdD2 | AT50 | modified CVB3 | hEPO | AU50 |
| Ana2-1 | AU50 | WTEMCV | hEPO | None |
| Ana2-2 | AU120 | modified CVB3 | hEPO | AU120 |

TABLE 11
Details of constructs tested for circularization

| | Subtype | 1 | 2 | Modification |
|---|---|---|---|---|
| Anabaena | IC3 | 5 nts | 12 nts | 4 nts |
| NrdB | IA2 | 13 nts | 17 nts | 4 nts (with |
| | | (partial) | (complete) | 1 insertion) |
| NrdD | IA2 | 8 nts | 17 nts | 4 nts (with |
| | | (partial) | (complete) | 1 insertion) |

NrdB and NrdD sequences are shown above, in Example 1. Ana2-1 and Ana2-2 contained identical intron-exon sequences, as follows:

Ana2: 3′ half-intron-3′ exon fragment
(SEQ ID NO: 46)
AACAACAGATAACTTACAGCTAGTCGGAAGGTGCAGAGACTCGACGGGA
GCTACCCTAACGTCAAGACGAGGGTAAAGAGAGAGTCCAATTCTCAAAG
CCAATAGGCAGTAGCGAAAGCTGCGGGAGAATGAAAATCCGTAGCGTCT
AAACGGTCGTGTGGGTTCAAGTCCCTCCACCCCCA
Ana2: 3′ half-intron
(SEQ ID NO: 47)
AACAACAGATAACTTACAGCTAGTCGGAAGGTGCAGAGACTCGACGGGA
GCTACCCTAACGTCAAGACGAGGGTAAAGAGAGAGTCCAATTCTCAAAG
CCAATAGGCAGTAGCGAAAGCTGCGGGAGAATG
Ana2: 3′ exon fragment
(SEQ ID NO: 48)
AAAATCCGTAGCGTCTAAACGGTCGTGTGGGTTCAAGTCCCTCCACCCC
CA
Ana2: 5′ exon fragment-5′ half-intron
(SEQ ID NO: 49)
AGACGCTACGGACTTAAATAATTGAGCCTTAGAGAAGAAATTCTTTAAG
TGGATGCTCTCAAACTCAGGGAAACCTAAATCTAGCTATAGACAAGGCA
ATCCTGAGCCAAGCCGAAGTAGTAATTAGTAAGTT
Ana2: 5′ exon fragment
(SEQ ID NO: 50)
AGACGCTACGGACTT
Ana2: 5′ half-intron
(SEQ ID NO: 51)
AAATAATTGAGCCTTAGAGAAGAAATTCTTTAAGTGGATGCTCTCAAAC
TCAGGGAAACCTAAATCTAGCTATAGACAAGGCAATCCTGAGCCAAGCC
GAAGTAGTAATTAGTAAGTT

Unmodified linear RNA was synthesized by in vitro transcription using T7 RNA polymerase from a DNA template in the presence of 7.5 mM of NTP. Template DNA was removed by treating with DNase for 20 minutes. Synthesized linear RNA was purified with an RNA clean up kit (New England Biolabs, T2050). Self-splicing occurred during transcription; no additional reaction was required. To monitor self-splicing efficiency, column purified, in vitro transcribed RNA was separated on an anionic exchange (AEX) column through HPLC. The percentage of linear and circular peaks were taken, and circularization efficiency was normalized with that of original constructs.

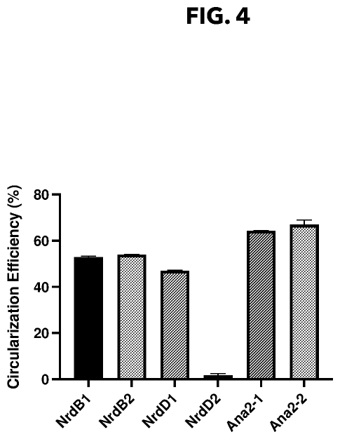

Extending the annealing sequence from 13 nts to 17 nts of the T4 phage nrdB self-splicing PIE (nrdB2) showed similar circularization efficiency with the nrdB1 PIE (nrdB1) and nrdD1 PIE (FIG. 4). This data indicates that circularization was not disrupted by extension of the annealing sequence in T4 phage nrdB self-splicing PIE. Further, the nrdB1, nrdB2, and nrdD1 constructs showed similar circularization efficiency as the Anabaena (Ana2-1 and Ana2-2) constructs that have extended annealing region.

Extending the annealing region of the T4 phage nrdD self-splicing PIE from 10 nts to 17 nts (nrdD2) significantly reduced circularization efficiency when compared with the nrdD PIE (nrdD1) (FIG. 4).

### Example 5: Immune Response Generated by T4 Phage nrdB or nrdD Self-Splicing Permuted Intron-Exon (PIE)

This example describes an immune response generated by circular RNA constructs generated by T4 phage nrdB or nrdD self-splicing PIE.

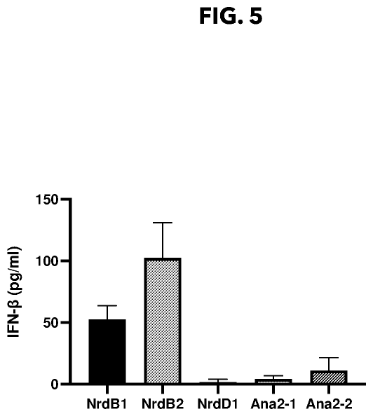

The circular RNA constructs produced in Example 4 were screened for differences in IFN-β response. A549 WT cells were transfected with in vitro transcribed RNA (60K/well), 0.2 μmol total RNA. Cells were collected after 24 hours. As shown in FIG. 5, nrdD1 construct showed a reduced immune response compared to other constructs (nrdB1 and nrdB2) and comparable immune response to Anabaena constructs (Ana2-1 and Ana2-2) in A549 cells.

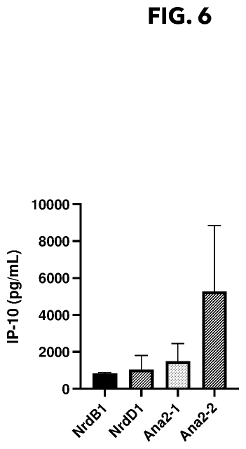

The circular RNA constructs produced in Example 4 were also screen for differences in IP-10 response. Primary macrophages were transfected with in vitro transcribed RNA (30K/well), 5 fmol total RNA. Cells were collected after 24 hours. As shown in FIG. 6, nrdB1 and nrdD1 constructs showed a reduced immune response compared to the Ana2-2 construct and a similar immune response to the Ana2-1 construct in macrophages.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

1. A linear polyribonucleotide having the formula 5′-(A)-(B)-(C)-(D)-(E)-(F)-(G)-3′, wherein:
(A) comprises a 3′ half of Group I catalytic intron fragment from a T4 phage nrdB gene or nrdD gene;
(B) comprises a 3′ splice site;
(C) comprises a 3′ exon fragment;
(D) comprises a polyribonucleotide cargo;
(E) comprises a 5′ exon fragment;
(F) comprises a 5′ splice site; and
(G) comprises a 5′ half of Group I catalytic intron fragment from a T4 phage nrdB gene or nrdD gene;
and wherein (A), (B), or (C) comprises a first annealing region comprising from 4 to 50 ribonucleotides and (E), (F), or (G) comprises a second annealing region comprising from 4 to 50 ribonucleotides,
wherein the first annealing region and the second annealing region have from 80% to 100% complementarity, or
the first annealing region and the second annealing region comprises from zero to 10 mismatched base pairs.

2. The linear polyribonucleotide of claim 1, wherein the first annealing region comprises from 6 to 30 ribonucleotides and the second annealing region comprises from 6 to 30 ribonucleotides.

3. The linear polyribonucleotide of claim 2, wherein the first annealing region comprises from 8 to 20 ribonucleotides and the second annealing region comprises from 8 to 20 ribonucleotides.

4. The linear polyribonucleotide of claim 3, wherein the first annealing region comprises from 8 to 17 ribonucleotides and the second annealing region comprises from 8 to 17 ribonucleotides.

5. The linear polyribonucleotide of claim 4, wherein the first annealing region comprises from 10 to 15 ribonucleotides and the second annealing region comprises from 13 to 17 ribonucleotides.

6. The linear polyribonucleotide of any one of claims 1-5, wherein the first annealing region and the second annealing region has 90% to 100% complementarity.

7. The linear polyribonucleotide of any one of claims 1-6, wherein the first annealing region and the second annealing region comprise zero or one mismatched base pair.

8. The linear polyribonucleotide of any one of claims 1-7, wherein the first annealing region and the second annealing region are 100% complementary.

9. The linear polyribonucleotide of any one of claims 1-8, wherein the 3′ exon fragment of (C) comprises the first annealing region and the 5′ exon fragment of (E) comprises the second annealing region.

10. The linear polyribonucleotide of any one of claims 1-9, wherein the 3′ half of Group I catalytic intron fragment of (A) is from the T4 phage nrdB gene.

11. The linear polyribonucleotide of claim 10, wherein the 3′ half of Group I catalytic intron fragment of (A) comprises a sequence having at least 80% sequence identity to 5′-TTGCAAAACAAGGTTCAACGACTAGTCTTCGGACGTAGGGTCAAGCGACTCGAAATGGGGAGAATC CCTCCGGGATTGTGATATAGTCTGGACTGCATGGTAACATGCAGCAGTTCATAAGAGAACGGGTTGA GAATTAGCGAGCTCAATCGAACATACG-3′ (SEQ ID NO: 2).

12. The linear polyribonucleotide of claim 11, wherein the 3′ half of Group I catalytic intron fragment of (A) comprises a sequence having at least 85%, 90%, 95%, 97%, 99%, or 100% sequence identity to 5′-TTGCAAAACAAGGTTCAACGACTAGTCTTCGGACGTAGGGTCAAGCGACTCGAAATGGGGAGAATC CCTCCGGGATTGTGATATAGTCTGGACTGCATGGTAACATGCAGCAGTTCATAAGAGAACGGGTTGA GAATTAGCGAGCTCAATCGAACATACG-3′ (SEQ ID NO: 2).

13. The linear polyribonucleotide of any one of claims 1-12, wherein the 5′ half of Group I catalytic intron fragment of (G) is from the T4 phage nrdB gene.

14. The linear polyribonucleotide of claim 13, wherein the 5′ half of Group I catalytic intron of (G) comprises a sequence having at least 80% sequence identity to 5′-AAAATGCGCCTTTAAACGGTAACGTTTATCGAAAACTCCTTTAATTGCTGGAAAGTCCTTTATGGAAA ACTAGCAGCCAAGGTTTTGCTT-3′ (SEQ ID NO: 6).

15. The linear polyribonucleotide of claim 14, wherein the 5′ half of Group I catalytic intron of (G) comprises a sequence having at least 85%, 90%, 95%, 97%, 99%, or 100% sequence identity to 5′-AAAATGCGCCTTTAAACGGTAACGTTTATCGAAAACTCCTTTAATTGCTGGAAAGTCCTTTATGGAAA ACTAGCAGCCAAGGTTTTGCTT-3′ (SEQ ID NO: 6).

16. The linear polyribonucleotide of any one of claims 1-9, wherein the 3′ half of Group I catalytic intron fragment of (A) is from the T4 phage nrdD gene.

17. The linear polyribonucleotide of claim 16, wherein the 3′ half of Group I catalytic intron fragment of (A) comprises a sequence having at least 80% sequence identity to 5′-CAGTAGCTGTAAATGCCCAACGACTATCCCTGATGAATGTAAGGGAGTAGGGTCAAGCGACCCGAA ACGGCAGACAACTCTAAGAGTTGAAGATATAGTCTGAACTGCATGGTGACATGCAGCTGTTTATCCT CGTATAAATATGAATACGAGGTGAAACGATGAAATGAATTACATTGTTTCATATAAACGGGTAGAGAA GTAGCGAACTCTACTGAACACATTG-3′ (SEQ ID NO: 10).

18. The linear polyribonucleotide of claim 17, wherein the 3′ half of Group I catalytic intron fragment of (A) comprises a sequence having at least 85%, 90%, 95%, 97%, 99%, or 100% sequence identity to 5′-CAGTAGCTGTAAATGCCCAACGACTATCCCTGATGAATGTAAGGGAGTAGGGTCAAGCGACCCGAA ACGGCAGACAACTCTAAGAGTTGAAGATATAGTCTGAACTGCATGGTGACATGCAGCTGTTTATCCT CGTATAAATATGAATACGAGGTGAAACGATGAAATGAATTACATTGTTTCATATAAACGGGTAGAGAA GTAGCGAACTCTACTGAACACATTG-3′ (SEQ ID NO: 10).

19. The linear polyribonucleotide of any one of claims 16-18, wherein the 5′ half of Group I catalytic intron fragment of (G) is from the T4 phage nrdD gene.

20. The linear polyribonucleotide of claim 19, wherein the 5′ half of Group I catalytic intron of (G) comprises a sequence having at least 80% sequence identity to 5′-TAACGTAAGTCAAGCTCATGTAAAATCTGCCTAAAACGGGAAACTCTCACTGAGACAATCCGTTGCTA AATCAG-3′ (SEQ ID NO: 14).

21. The linear polyribonucleotide of claim 20, wherein the 5′ half of Group I catalytic intron of (G) comprises a sequence having at least 85%, 90%, 95%, 97%, 99%, or 100% sequence identity to 5′-TAACGTAAGTCAAGCTCATGTAAAATCTGCCTAAAACGGGAAACTCTCACTGAGACAATCCGTTGCTA AATCAG-3′ (SEQ ID NO: 14).

22. The linear polyribonucleotide of any one of claims 1-21, wherein the 3′ exon fragment of (C) comprises a sequence having at least 80% sequence identity to 5′-GTACCTTTAACTTCCATAAGAACATGGAAATCATGGAAGGTAATGCCAAG-3′ (SEQ ID NO: 3).

23. The linear polyribonucleotide of claim 22, wherein the 3′ exon fragment of (C) comprises a sequence having at least 85%, 90%, 95%, 97%, 99%, or 100% sequence identity to 5′-GTACCTTTAACTTCCATAAGAACATGGAAATCATGGAAGGTAATGCCAAG-3′ (SEQ ID NO: 3).

24. The linear polyribonucleotide of any one of claims 1-21, wherein the 3′ exon fragment of (C) comprises a sequence having at least 80% sequence identity to 5′-GTACCTTTAACTTCCAAAAGATACATAAAAATCATGGAAGGTAATGCCAAG-3′ (SEQ ID NO: 8).

25. The linear polyribonucleotide of claim 24, wherein the 3′ exon fragment of (C) comprises a sequence having at least 85%, 90%, 95%, 97%, 99%, or 100% sequence identity to 5′-GTACCTTTAACTTCCAAAAGATACATAAAAATCATGGAAGGTAATGCCAAG-3′ (SEQ ID NO: 8).

26. The linear polyribonucleotide of any one of claims 22-25, wherein the 5′ exon fragment of (E) comprises a sequence having at least 80% sequence identity to 5′-TTTTTATGTATOTTTTGCGT-3′ (SEQ ID NO: 5).

27. The linear polyribonucleotide of claim 26, wherein the 5′ exon fragment of (E) comprises a sequence having at least 85%, 90%, 95%, 97%, 99%, or 100% sequence identity to 5′-TTTTTATGTATOTTTTGCGT-3′ (SEQ ID NO: 5).

28. The linear polyribonucleotide of any one of claims 1-21, wherein the 3′ exon fragment of (C) comprises a sequence having at least 80% sequence identity to 5′-ATGAAGTGAACACGTTATTCAGTTCAAACGGACAGACTCCTTTTGTAACA-3′ (SEQ ID NO: 11).

29. The linear polyribonucleotide of claim 28, wherein the 3′ exon fragment of (C) comprises a sequence having at least 85%, 90%, 95%, 97%, 99%, or 100% sequence identity to 5′-ATGAAGTGAACACGTTATTCAGTTCAAACGGACAGACTCCTTTTGTAACA-3′ (SEQ ID NO: 11).

30. The linear polyribonucleotide of any one of claims 1-21, wherein the 3′ exon fragment of (C) comprises a sequence having at least 80% sequence identity to 5′-ATGAAGTGAACACGTTACATAAGCTTGGAATGCAGACTCCTTTTGTAACA-3′ (SEQ ID NO: 16).

31. The linear polyribonucleotide of claim 30, wherein the 3′ exon fragment of (C) comprises a sequence having at least 85%, 90%, 95%, 97%, 99%, or 100% sequence identity to 5′-ATGAAGTGAACACGTTACATAAGCTTGGAATGCAGACTCCTTTTGTAACA-3′ (SEQ ID NO: 16).

32. The linear polyribonucleotide of any one of claims 28-31, wherein the 5′ exon fragment of (E) comprises a sequence having at least 80% sequence identity to 5′-TGCATTCCAAGCTTATGAGT-3′ (SEQ ID NO: 13).

33. The linear polyribonucleotide of claim 32, wherein the 5′ exon fragment of (E) comprises a sequence having at least 85%, 90%, 95%, 97%, 99%, or 100% sequence identity to 5′-TGCATTCCAAGCTTATGAGT-3′ (SEQ ID NO: 13).

34. The linear polyribonucleotide of any one of claims 1-33, wherein the 3′ half of Group I catalytic intron fragment of (A) is the 5′ terminus of the linear polynucleotide.

35. The linear polyribonucleotide of any one of claims 1-34, wherein the 5′ half of Group I catalytic intron fragment of (G) is the 3′ terminus of the linear polyribonucleotide.

36. The linear polyribonucleotide of any one of claims 1-35, wherein the linear polyribonucleotide does not comprise a further annealing region.

37. The linear polyribonucleotide any one of claims 1-36, wherein the linear polyribonucleotide does not comprise an annealing region 3′ to (A) that comprises partial or complete nucleic acid complementarity with an annealing region 5′ to (G).

38. The linear polyribonucleotide any one of claims 1-37, wherein the polyribonucleotide cargo of (D) comprises an expression sequence, a non-coding sequence, or an expression sequence and a non-coding sequence.

39. The linear polyribonucleotide any one of claims 1-38, wherein the polyribonucleotide cargo of (D) comprises an expression sequence encoding a polypeptide.

40. The linear polyribonucleotide any one of claims 1-39, wherein the polyribonucleotide cargo of (D) comprises an IRES operably linked to an expression sequence encoding a polypeptide.

41. The linear polyribonucleotide any one of claims 1-40, wherein the polyribonucleotide cargo of (D) comprises an expression sequence that encodes a polypeptide that has a biological effect on a subject.

42. The linear polyribonucleotide of any one of claims 1-41, wherein the linear polyribonucleotide further comprises a first spacer region between the 3′ exon fragment of (C) and the polyribonucleotide cargo of (D).

43. The linear polyribonucleotide of any one of claims 1-42, wherein the linear polyribonucleotide further comprises a second spacer region between the polyribonucleotide cargo of (D) and the 5′ exon fragment of (E).

44. The linear polyribonucleotide of claim 42 or 43, wherein each spacer region is at least 5 ribonucleotides in length.

45. The linear polyribonucleotide of claim 44, wherein each spacer region is from 5 to 500 ribonucleotides in length.

46. The linear polyribonucleotide of any one of claims 43-45, wherein the first spacer region, the second spacer region, or the first spacer region and the second spacer region comprises a polyA sequence.

47. The linear polyribonucleotide of any one of claims 42-46, wherein the first spacer region, the second spacer region, or the first spacer region and the second spacer region comprises a polyA-C sequence.

48. The linear polyribonucleotide of any one of claims 1-47, wherein the linear polyribonucleotide is from 300 to 20,000 ribonucleotides in length.

49. The linear polyribonucleotide of any one of claims 1-48, wherein the linear polyribonucleotide is at least 1,000 ribonucleotides in length.

50. The linear polyribonucleotide of claim 49, wherein the linear polyribonucleotide is at least 3,000 ribonucleotides in length.

51. A DNA vector comprising an RNA polymerase promoter operably linked to a DNA sequence that encodes the linear polyribonucleotide of any one of claims 1-50.

52. A circular polyribonucleotide produced from the linear polyribonucleotide of any one of claims 1-50 or from the DNA vector of claim 51.

53. A method of expressing a polypeptide in a cell, the method comprising providing the linear polyribonucleotide of any one of claims 1-50, the DNA vector of claim 51, or the circular polyribonucleotide of claim 52 to the cell.

54. A method of producing a circular polyribonucleotide from the linear polyribonucleotide of any one of claims 1-50, the method comprising providing the linear polyribonucleotide under conditions suitable for self-splicing of the linear polyribonucleotide to produce the circular polyribonucleotide.


SEQUENCE LISTING (55 sequences)

SEQ ID NO: 1 - RNA, 210 nt, Escherichia virus T4
  source 1..210: mol_type = other RNA
ttgcaaaacaaggttcaacgactagtcttcggacgtagggtcaagcgactcgaaatggggagaatccctccgggattgtgatatagtctggactgcatggtaacatgcagcagttcataagagaacgggttgagaattagcgagctcaatcgaacatacggtacctttaacttccataagaacatggaaatcatggaaggtaatgccaag

SEQ ID NO: 2 - RNA, 160 nt, Escherichia virus T4
  source 1..160: mol_type = other RNA
ttgcaaaacaaggttcaacgactagtcttcggacgtagggtcaagcgactcgaaatggggagaatccctccgggattgtgatatagtctggactgcatggtaacatgcagcagttcataagagaacgggttgagaattagcgagctcaatcgaacatacg

SEQ ID NO: 3 - RNA, 50 nt, Escherichia virus T4
  source 1..50: mol_type = other RNA
gtacctttaacttccataagaacatggaaatcatggaaggtaatgccaag

SEQ ID NO: 4 - RNA, 110 nt, Escherichia virus T4
  source 1..110: mol_type = other RNA
tttttatgtatcttttgcgtaaaatgcgcctttaaacggtaacgtttatcgaaaactcctttaattgctggaaagtcctttatggaaaactagcagccaaggttttgctt

SEQ ID NO: 5 - RNA, 20 nt, Escherichia virus T4
  source 1..20: mol_type = other RNA
tttttatgtatcttttgcgt

SEQ ID NO: 6 - RNA, 90 nt, Escherichia virus T4
  source 1..90: mol_type = other RNA
aaaatgcgcctttaaacggtaacgtttatcgaaaactcctttaattgctggaaagtcctttatggaaaactagcagccaaggttttgctt

SEQ ID NO: 7 - RNA, 211 nt, Escherichia virus T4
  source 1..211: mol_type = other RNA
ttgcaaaacaaggttcaacgactagtcttcggacgtagggtcaagcgactcgaaatggggagaatccctccgggattgtgatatagtctggactgcatggtaacatgcagcagttcataagagaacgggttgagaattagcgagctcaatcgaacatacggtacctttaacttccaaaagatacataaaaatcatggaaggtaatgccaag

SEQ ID NO: 8 - RNA, 51 nt, Escherichia virus T4
  source 1..51: mol_type = other RNA
gtacctttaacttccaaaagatacataaaaatcatggaaggtaatgccaag

SEQ ID NO: 9 - RNA, 276 nt, Escherichia virus T4
  source 1..276: mol_type = other RNA
cagtagctgtaaatgcccaacgactatccctgatgaatgtaagggagtagggtcaagcgacccgaaacggcagacaactctaagagttgaagatatagtctgaactgcatggtgacatgcagctgtttatcctcgtataaatatgaatacgaggtgaaacgatgaaatgaattacattgtttcatataaacgggtagagaagtagcgaactctactgaacacattgatgaagtgaacacgttattcagttcaaacggacagactccttttgtaaca

SEQ ID NO: 10 - RNA, 226 nt, Escherichia virus T4
  source 1..226: mol_type = other RNA
cagtagctgtaaatgcccaacgactatccctgatgaatgtaagggagtagggtcaagcgacccgaaacggcagacaactctaagagttgaagatatagtctgaactgcatggtgacatgcagctgtttatcctcgtataaatatgaatacgaggtgaaacgatgaaatgaattacattgtttcatataaacgggtagagaagtagcgaactctactgaacacattg

SEQ ID NO: 11 - RNA, 50 nt, Escherichia virus T4
  source 1..50: mol_type = other RNA
atgaagtgaacacgttattcagttcaaacggacagactccttttgtaaca

SEQ ID NO: 12 - RNA, 94 nt, Escherichia virus T4
  source 1..94: mol_type = other RNA
tgcattccaagcttatgagttaacgtaagtcaagctcatgtaaaatctgcctaaaacgggaaactctcactgagacaatccgttgctaaatcag

SEQ ID NO: 13 - RNA, 20 nt, Escherichia virus T4
  source 1..20: mol_type = other RNA
tgcattccaagcttatgagt

SEQ ID NO: 14 - RNA, 74 nt, Escherichia virus T4
  source 1..74: mol_type = other RNA
taacgtaagtcaagctcatgtaaaatctgcctaaaacgggaaactctcactgagacaatccgttgctaaatcag

SEQ ID NO: 15 - RNA, 276 nt, Escherichia virus T4
  source 1..276: mol_type = other RNA
cagtagctgtaaatgcccaacgactatccctgatgaatgtaagggagtagggtcaagcgacccgaaacggcagacaactctaagagttgaagatatagtctgaactgcatggtgacatgcagctgtttatcctcgtataaatatgaatacgaggtgaaacgatgaaatgaattacattgtttcatataaacgggtagagaagtagcgaactctactgaacacattgatgaagtgaacacgttacataagcttggaatgcagactccttttgtaaca

SEQ ID NO: 16 - RNA, 50 nt, Escherichia virus T4
  source 1..50: mol_type = other RNA
atgaagtgaacacgttacataagcttggaatgcagactccttttgtaaca

SEQ ID NO: 17 - protein, 6 aa, synthetic construct
CHYSEL

SEQ ID NO: 18 - protein, 9 aa, synthetic construct
  VARIANT 1: note = absent or G or H
  VARIANT 2: note = absent or D or G
  VARIANT 3: note = D or V or I or S or M
  VARIANT 5: note = any amino acid
XXXEXNPGP

SEQ ID NO: 19 - protein, 8 aa, synthetic construct
  VARIANT 2: note = or I
  VARIANT 4: note = any amino acid
DVEXNPGP

SEQ ID NO: 20 - protein, 9 aa, synthetic construct
GDVESNPGP

SEQ ID NO: 21 - protein, 9 aa, synthetic construct
GDIEENPGP

SEQ ID NO: 22 - protein, 7 aa, synthetic construct
VEPNPGP

SEQ ID NO: 23 - protein, 7 aa, synthetic construct
IETNPGP

SEQ ID NO: 24 - protein, 9 aa, synthetic construct
GDIESNPGP

SEQ ID NO: 25 - protein, 9 aa, synthetic construct
GDVELNPGP

SEQ ID NO: 26 - protein, 9 aa, synthetic construct
GDIETNPGP

SEQ ID NO: 27 - protein, 8 aa, synthetic construct
GDVENPGP

SEQ ID NO: 28 - protein, 9 aa, synthetic construct
GDVEENPGP

SEQ ID NO: 29 - protein, 9 aa, synthetic construct
GDVEQNPGP

SEQ ID NO: 30 - protein, 7 aa, synthetic construct
IESNPGP

SEQ ID NO: 31 - protein, 9 aa, synthetic construct
GDIELNPGP

SEQ ID NO: 32 - protein, 9 aa, synthetic construct
HDIETNPGP

SEQ ID NO: 33 - protein, 9 aa, synthetic construct
HDVETNPGP

SEQ ID NO: 34 - protein, 9 aa, synthetic construct
HDVEMNPGP

SEQ ID NO: 35 - protein, 9 aa, synthetic construct
GDMESNPGP

SEQ ID NO: 36 - protein, 9 aa, synthetic construct
GDVETNPGP

SEQ ID NO: 37 - protein, 9 aa, synthetic construct
GDIEQNPGP

SEQ ID NO: 38 - protein, 8 aa, synthetic construct
DSEFNPGP

SEQ ID NO: 39 - protein, 10 aa, synthetic construct
  VARIANT 3: note = a purine (adenine or guanine)
  VARIANT 4: note = or G
CCXACCAUGG

SEQ ID NO: 40 - protein, 10 aa, synthetic construct
ATACGGTACC

SEQ ID NO: 41 - protein, 10 aa, synthetic construct
TGCGTAAAAT

SEQ ID NO: 42 - protein, 10 aa, synthetic construct
CATTGATGAA

SEQ ID NO: 43 - protein, 10 aa, synthetic construct
TGAGTTAACG

SEQ ID NO: 44 - protein, 741 aa, Coxsackie B virus 3
TTAAAACAGCCTGTGGGTTGATCCCACCCACAGGCCCATTGGGCGCTAGCACTCTGGTATCACGGTACCTTTGTGCGCCTGTTTTATACCCCCTCCCCCAACTGTAACTTAGAAGTAACACACACCGATCAACAGTCAGCGTGGCACACCAGCCACGTTTTGATCAAGCACTTCTGTTACCCCGGACTGAGTATCAATAGACTGCTCACGCGGTTGAAGGAGAAAGCGTTCGTTATCCGGCCAACTACTTCGAAAAACCTAGTAACACCGTGGAAGTTGCAGAGTGTTTCGCTCAGCACTACCCCAGTGTAGATCAGGTCGATGAGTCACCGCATTCCCCACGGGCGACCGTGGCGGTGGCTGCGTTGGCGGCCTGCCCATGGGGAAACCCATGGGACGCTCTAATACAGACATGGTGCGAAGAGTCTATTGAGCTAGTTGGTAGTCCTCCGGCCCCTGAATGCGGCTAATCCTAACTGCGGAGCACACACCCTCAAGCCAGAGGGCAGTGTGTCGTAACGGGCAACTCTGCAGCGGAACCGACTACTTTGGGTGTCCGTGTTTCATTTTATTCCTATACTGGCTGCTTATGGTGACAATTGAGAGATCGTTACCATATAGCTATTGGATTGGCCATCCGGTGACTAATAGAGCTATTATATATCCCTTTGTTGGGTTTATACCACTTAGCTTGAAAGAGGTTAAAACATTACAATTCATTGTTAAGTTGAATACAGCAAC

SEQ ID NO: 45 - protein, 552 aa, Enterovirus A71
  source 1..552: note = EV-A71
ACGTTACTGGCCGAAGCCGCTTGGAATAAGGCCGGTGTGCGTTTGTCTATATGTTATTTTCCACCATATTGCCGTCTTTTGGCAATGTGAGGGCCCGGAAACCTGGCCCTGTCTTCTTGACGAGCATTCCTAGGGGTCTTTCCCCTCTCGCCAAAGGAATGCAAGGTCTGTTGAATGTCGTGAAGGAAGCAGTTCCTCTGGAAGCTTCTTGAAGACAAACAACGTCTGTAGCGACCCTTTGCAGGCAGCGGAACCCCCCACCTGGCGACAGGTGCCTCTGCGGCCAAAAGCCACGTGTATAAGATACACCTGCAAAGGCGGCACAACCCCAGTGCCACGTTGTGAGTTGGATAGTTGTGGAAAGAGTCAAATGGCTCTCCTCAAGCGTATTCAACAAGGGGCTGAAGGATGCCCAGAAGGTACCCCATTGTATGGGATCTGATCTGGGGCCTCGGTGCACATGCTTTACATGTGTTTAGTCGAGGTTAAAAAACGTCTAGGCCCCCCGAACCACGGGGACGTGGTTTTCCTTTGAAAAACACGATGATAATA

SEQ ID NO: 46 - protein, 182 aa, Anabaena sp.
AACAACAGATAACTTACAGCTAGTCGGAAGGTGCAGAGACTCGACGGGAGCTACCCTAACGTCAAGACGAGGGTAAAGAGAGAGTCCAATTCTCAAAGCCAATAGGCAGTAGCGAAAGCTGCGGGAGAATGAAAATCCGTAGCGTCTAAACGGTCGTGTGGGTTCAAGTCCCTCCACCCCCA

SEQ ID NO: 47 - protein, 131 aa, Anabaena sp.
AACAACAGATAACTTACAGCTAGTCGGAAGGTGCAGAGACTCGACGGGAGCTACCCTAACGTCAAGACGAGGGTAAAGAGAGAGTCCAATTCTCAAAGCCAATAGGCAGTAGCGAAAGCTGCGGGAGAATG

SEQ ID NO: 48 - protein, 51 aa, Anabaena sp.
AAAATCCGTAGCGTCTAAACGGTCGTGTGGGTTCAAGTCCCTCCACCCCCA

SEQ ID NO: 49 - protein, 133 aa, Anabaena sp.
AGACGCTACGGACTTAAATAATTGAGCCTTAGAGAAGAAATTCTTTAAGTGGATGCTCTCAAACTCAGGGAAACCTAAATCTAGCTATAGACAAGGCAATCCTGAGCCAAGCCGAAGTAGTAATTAGTAAGTT

SEQ ID NO: 50 - protein, 15 aa, Anabaena sp.
AGACGCTACGGACTT

SEQ ID NO: 51 - protein, 118 aa, Anabaena sp.
AAATAATTGAGCCTTAGAGAAGAAATTCTTTAAGTGGATGCTCTCAAACTCAGGGAAACCTAAATCTAGCTATAGACAAGGCAATCCTGAGCCAAGCCGAAGTAGTAATTAGTAAGTT

SEQ ID NO: 52 - protein, 16 aa, Escherichia virus T4
CATAAGAACATGGAAA

SEQ ID NO: 53 - protein, 17 aa, Escherichia virus T4
CAAAAGATACATAAAAA

SEQ ID NO: 54 - protein, 17 aa, Escherichia virus T4
TTCAGTTCAAACGGACA

SEQ ID NO: 55 - protein, 17 aa, Escherichia virus T4
CATAAGCTTGGAATGCA